(19) United States
(12) Patent Application Publication
(10) Pub. No.: US 2020/0375750 A1
(43) Pub. Date: Dec. 3, 2020

(54) SYSTEMS AND METHODS FOR FUSION OF SACROILIAC JOINT

(71) Applicant: Advance Research System, LLC, Edina, MN (US)

(72) Inventors: Hamid R. Abbasi, Edina, MN (US); Kenneth R. Barra, Dallas, GA (US); Stuart J. Olstad, Plymouth, MN (US)

(21) Appl. No.: 16/902,118

(22) Filed: Jun. 15, 2020

Related U.S. Application Data
(63) Continuation-in-part of application No. 16/443,303, filed on Jun. 17, 2019.
(60) Provisional application No. 62/970,991, filed on Feb. 6, 2020.
(60) Provisional application No. 62/861,937, filed on Jun. 14, 2019.
(60) Provisional application No. 62/861,937, filed on Jun. 14, 2019.
(60) Provisional application No. 62/801,316, filed on Feb. 5, 2019.
(60) Provisional application No. 62/685,605, filed on Jun. 15, 2018.

(51) Int. Cl. A61F 2/30

(52) U.S. Cl. CPC A61F 2/30988; A61F 2002/30995; A61B 17/8615; A61F 2002/30858; A61F 2002/30622

(57) ABSTRACT

Systems for performing a minimally invasive sacroiliac joint fusion. The system may be in the form of a disposable kit, with the components streamlined so that the procedure can be performed in a few minutes. The screw components are self-drilling and self-tapping. The system may deploy blades through the walls of the primary screw which cut away material as the primary screw is set, for denuding the sacroiliac joint. The primary screws are designed to bore through and internalize bone tissue in an autografting process. The implant system may include components for packing bone grafting material into the screw to supplement autograft bone tissue internalized in the primary screw during placement. At least one side screw is passed through a head of the primary screw to anchor the head and inhibit rotation (backing out) after implantation. The primary screw may include features that facilitate rotational alignment.

### RELATED APPLICATIONS

This patent application is a continuation-in-part of U.S. patent application Ser. No. 16/443,303, filed Jun. 17, 2019, which claims the benefit of U.S. Provisional Patent Application No. 62/861,937 filed Jun. 14, 2019, and U.S. Provisional Patent Application No. 62/801,316, filed Feb. 5, 2019 and U.S. Provisional Patent Application No. 62/685,605, filed Jun. 15, 2018. This patent application also claims the benefit of U.S. Provisional Patent Application No. 62/861,937, filed Jun. 14, 2019, and of U.S. Provisional Patent Application No. 62/970,991, filed Feb. 6, 2020. The disclosures of these related applications are hereby incorporated by reference herein in their entireties.

### FIELD OF THE DISCLOSURE

The present disclosure is directed generally to tools and techniques for bone fusion, and more specifically to apparatuses and methods for fusion of a sacroiliac joint.

### BACKGROUND OF THE DISCLOSURE

Products and techniques for fusion of sacroiliac joints are known. Many techniques involve the implantation of a bone screw that extends substantially perpendicular to the joint. Conventional implantation techniques may require about an hour of surgery to perform. Also, the implants have been known to fail, requiring removal of the implanted bone screw and redress of the joint, at considerable cost and discomfort to the patient. An implantation system that reduces surgical time with improved outcomes would be welcomed.

### SUMMARY OF THE DISCLOSURE

Various embodiments of the disclosure include an implant system for performing a minimally invasive sacroiliac joint fusion. The system may be in the form of a disposable kit, with the components augmenting a streamlined procedure that can be performed in under ten minutes. The screw components are self-drilling and self-tapping, thus requiring no pre-drilling, and can be performed without cannulation.

Many conventional fusion systems for sacroiliac joints involve pre-drilling a passage through the ilium and into the sacrum at an approach that is substantially normal to the joint. A bone screw is then implanted in the pre-drilled passage. The pre-drilled passage is sized so that a root diameter of the threads of the bone screw cause an interference fit with the pre-drilled passage, while the protruding portions of the thread cut into the bone. The interference fit in addition to the cutting depth of the threads into the bone anchor the bone screw into place. Often, the surgical site is augmented with growth-promoting biologic to help replace the bone removed from the pre-drilled passage.

There are certain disadvantages that are inherent to the conventional method described above. First, it is noted that live bone tissue is generally compressible or “spongy”, particularly underneath the harder cortical exterior. The threads implanted by the conventional method described above may be readily stripped from such tissue, which can negate the benefit of the implant.

Second, such conventional procedures remove large portions of the pre-drilled bone. As such, conventional procedures do not take full advantage of natural live autograft bone tissue, which is a better growth-promoting substance than other options, such as allograft, synthetic biologic, or xenograft biologic.

Third, conventional procedures can require several surgical approaches and attendant reconfigurations, thereby increasing surgery time. For example, an implantation that involves placement of a primary bone screw plus a pair of side screws to secure the bone screw essentially involves three surgeries: one for placement of the primary screw and one for each of the two side screws. The placement of each component requires reconfiguration to properly align the component.

The implant system of the present disclosure is based on different principles of operation. The bone screw of the disclosed implant system does not require the separate step of pre-drilling a bore for placement of the primary bone screw. Instead, the self-tapping distal tip of the disclosed primary screw acts as a pre-drill. The disclosed implant displaces a significant portion of the bone tissue radially inward, capturing (internalizing) the tissue within an interior chamber of the bone screw. The autograft bone tissue lodged within the bone screw augments the bone growth characteristics of any allograft, xenograft, or synthetic biologic that may be required. In some embodiments, the internally lodged bone tissue sufficiently fills the interior chamber so that addition of biologic is not necessary. The operating principle is to utilize more autograft bone tissue than conventional procedures to increase the effectiveness of the fusion and improve surgical outcomes.

The self-tapping aspect of the primary screw also takes advantage of the spongy, compliant nature of the bone tissue by displacing the remaining portion of the tissue radially outward, which compresses the bone within and around the threads. With the threads imbedded in tissue of greater density, the risk stripping or displacement of the bone screw is diminished.

The components of the disclosed system are designed to streamline the placement of the primary screw and one or more side screws with a single approach. That is, the side screw(s) are implanted without need for placement or alignment of a separate fixture.

In some embodiments, the bone screw is configured to enable the threads to extend to or very close to the head of the bone screw. This enhances the grip of the threads at the harder, denser cortical bone tissue at the exterior of the bone that registers against the head of the bone screw.

Some embodiments of the implant system enables the selective deployment of blades through ports formed in the walls of the primary screw to cut away material at selected depths (e.g., from the cartilage between the ilium and sacrum) as the primary screw is set, for denuding the sacroiliac joint. In some embodiments, the implant system includes components for packing bone grafting material into the screw to supplement the autograft bone tissue internalized by the primary screw and distributing into a zone or zones external to the primary screw created by the blades. The zone external to the primary screw may be annular and surround the primary screw. At least one side screw may be passed through a head of the primary screw to anchor the head and prevent it from backing out after implantation. Some embodiments of the side screws implement a passive locking mechanism when mounted to the bone screw that rotationally secures the side screw to prevent back out or loosening. Various embodiments of the disclosure also provide capabilities for retention of the side screw driver to the side screws, as well as provisions for retrieval of the side screw. The side screws may also include at least one side cavity that extends over a majority of the threaded length of the side screw, providing a channel through which bone tissue may grow for enhancement of fusion across bone interfaces.

Various configurations and methods are disclosed that assist the operator in rotationally aligning the primary screw and the corresponding side screw ports for improved and precise placement of the side screws. Rotational alignment is accomplished by the use of features and/or relative location of side ports formed in the body of the primary screw. In some embodiments, the alignment configurations and techniques takes advantage of the semi-transparency of various materials of the primary screw (e.g., titanium) to facilitate the alignment.

### BRIEF DESCRIPTION OF THE DRAWINGS

FIG. 1 is a perspective view of components of an implant system for fusion of a sacroiliac joint according to an embodiment of the disclosure;

FIG. 2 is a proximal perspective view of a primary screw according to an embodiment of the disclosure;

FIG. 3 is a distal perspective view of the primary screw of FIG. 2 according to an embodiment of the disclosure;

FIG. 4 is a first side elevational view of the primary screw of FIG. 2 according to an embodiment of the disclosure;

FIG. 5 is a first side sectional view of the primary screw of FIG. 2 according to an embodiment of the disclosure;

FIG. 6 is a second side elevational view of the primary screw of FIG. 2 according to an embodiment of the disclosure;

FIG. 6A is a second side sectional view of the primary screw of FIG. 2 according to an embodiment of the disclosure;

FIG. 7 is an elevational view of the primary screw of FIG. 2 to illustrate a tapered angle of the thread radii according to an embodiment of the disclosure;

FIG. 8 is an elevational view of a primary screw having elongated side ports centered about lateral axes that are offset relative to a mid-plane of the primary screw according to an embodiment of the disclosure;

FIG. 8A is a sectional view of the primary screw of FIG. 8 at plane VIII-VIII with elongated side ports having parallel edge walls according to an embodiment of the disclosure;

FIG. 8B is a sectional view of the primary screw of FIG. 8 at plane VIII-VIII with each elongated side port having an inclined edge wall according to an embodiment of the disclosure;

FIG. 9 is an elevational view of a primary screw having threads of varying pitch and crest dimensions along a body portion according to an embodiment of the disclosure;

FIGS. 10A and 10B are a schematic sectional views of primary screws having angled threads according to an embodiment of the disclosure;

FIG. 11 is a proximal perspective view of a primary screw having a flange with external notches according to an embodiment of the disclosure;

FIG. 12A is a proximal perspective view of an inserter according to an embodiment of the disclosure;

FIG. 12B is a distal perspective view of the inserter of FIG. 12A according to an embodiment of the disclosure;

FIG. 13 is side elevational view of the inserter of FIG. 12A identifying cross-sections XIV-XIV and XXI-XXI according to an embodiment of the disclosure;

FIG. 14 is a partial sectional view along cross-section XIV-XIV of FIG. 13 according to an embodiment of the disclosure;

FIG. 15 is an end view of the inserter of FIG. 12A identifying cross-section XVI-XVI according to an embodiment of the disclosure;

FIG. 16 is a sectional view along cross-section XVI-XVI of FIG. 15 according to an embodiment of the disclosure;

FIG. 17 is an end view of the inserter of FIG. 12A identifying cross-section XVIII-XVIII according to an embodiment of the disclosure;

FIG. 18 is a sectional view along cross-section XVIII-XVIII of FIG. 17 according to an embodiment of the disclosure;

FIG. 19 is an end view of the inserter of FIG. 12A identifying cross-section XX-XX according to an embodiment of the disclosure;

FIG. 20 is a sectional view along cross-section XX-XX of FIG. 19 according to an embodiment of the disclosure;

FIG. 21 is a sectional view along cross-section XXI-XXI of FIG. 13 according to an embodiment of the disclosure;

FIG. 22 is an enlarged partial view of a distal end of a guide rod of FIG. 1 according to an embodiment of the disclosure;

FIG. 23 is an enlarged partial view of a proximal end of a guide rod of FIG. 1 according to an embodiment of the disclosure;

FIG. 24 is a perspective longitudinal sectional view of a primary screw driver of FIG. 1 according to an embodiment of the disclosure;

FIG. 25 is a perspective view of a blade assembly of FIG. 1 in isolation;

FIG. 26 is an enlarged partial view of the proximal end of the blade assembly of FIG. 25 according to an embodiment of the disclosure;

FIG. 27 is an enlarged partial view of a transition between a proximal portion and a distal portion of a blade of the proximal end of the blade assembly of FIG. 25 according to an embodiment of the disclosure;

FIG. 28 is an enlarged perspective view of a side screw of FIG. 1 in isolation;

FIG. 28A is an enlarged perspective view of the side screw of FIG. 28 with side cavities according to an embodiment of the disclosure;

FIG. 28B is an enlarged sectional view of the side screw of FIG. 28A at plane B-B according to an embodiment of the disclosure;

FIG. 29 is a partial, enlarged view of a proximal end of the side screw of FIG. 28 according to an embodiment of the disclosure;

FIG. 30 is an enlarged, partial perspective view of a proximal end of a side screw driver of FIG. 1 according to an embodiment of the disclosure;

FIG. 31 is an enlarged, partial perspective view of a distal end of a side screw driver of FIG. 1 according to an embodiment of the disclosure;

FIG. 32 is an end view of a side screw with an oblong head initially contacting an oblong side screw port of a primary screw according to an embodiment of the disclosure;

FIG. 32A is a partial sectional view of the side screw and side screw port along plane XXXIIA-XXXIIA of FIG. 32 according to an embodiment of the disclosure;

FIG. 33 is an end view of the side screw and side screw port of FIG. 32 with the side screw fully seated within the side screw port according to an embodiment of the disclosure;

FIG. 33A is a partial sectional view of the side screw and side screw port along plane XXXIIIA-XXXIIIA of FIG. 33 according to an embodiment of the disclosure;

FIG. 34A is a partial sectional view of a side screw with a detent initially contacting a side screw port with a groove for receiving the detent according to an embodiment of the disclosure;

FIG. 34B is a partial sectional view of the side screw fully seated within the side screw port of FIG. 34A according to an embodiment of the disclosure;

FIG. 35A is a lower perspective view of a primary screw having a breach in the flange according to an embodiment of the disclosure;

FIG. 35B is an upper perspective view of the primary screw of FIG. 35A as viewed along a side screw port axis according to an embodiment of the disclosure;

FIG. 36 is a top view of a side screw according to an embodiment of the disclosure;

FIG. 36A is a side view from a perspective A of FIG. 36 according to an embodiment of the disclosure;

FIG. 36B is a side view from a perspective B of FIG. 36 according to an embodiment of the disclosure;

FIG. 37A is an enlarged, partial perspective view of FIG. 35B with the side screw of FIG. 36 disposed within a side screw port and having a major dimension of oblong threads in engagement with female threads of the side screw port according to an embodiment of the disclosure;

FIG. 37B is an enlarged, partial perspective view of FIG. 35B with the side screw of FIG. 36 disposed within a side screw port in an equipoise position, having a minor dimension of oblong threads in engagement with female threads of the side screw port according to an embodiment of the disclosure;

FIG. 38 is an enlarged, sectional view of a drive cap of FIG. 1 according to an embodiment of the disclosure;

FIG. 39 is a partial, enlarged sectional view of a proximal end of a plunger of FIG. 1 according to an embodiment of the disclosure;

FIG. 40 is an enlarged perspective view of a first side of a multifunctional handle according to an embodiment of the disclosure;

FIG. 41 is an enlarged perspective view of a second side of the multifunctional handle of FIG. 40 according to an embodiment of the disclosure;

FIG. 42 is an elevational, sectional view of an initial assembly of the primary screw, inserter, primary screw driver, blade assembly, and drive cap of FIG. 1 according to an embodiment of the disclosure;

FIG. 43 is a partial sectional view of the initial assembly of the primary screw, inserter, and primary screw driver of FIG. 1 orthogonal to the sectional view of FIG. 42 according to an embodiment of the disclosure;

FIG. 44 is an enlarged, perspective cutaway view of a proximal end of the initial assembly of FIG. 42 according to an embodiment of the disclosure;

FIG. 45 is an elevational view of the guide rod of FIG. 22 in operation according to an embodiment of the disclosure;

FIG. 46 is an elevational view of the initial assembly of FIG. 42 in operation over the guide rod of FIG. 45 according to an embodiment of the disclosure;

FIG. 47 is an elevational view of the initial assembly of FIG. 42 with the blades deployed during implantation of the primary screw according to an embodiment of the disclosure;

FIG. 48 is a schematic view of a surgical imaging device for rotationally aligning an implanted primary screw according to an embodiment of the disclosure;

FIG. 48A is a three-dimensional image of aligned elongate side ports for determining the orientation of the primary screw according to an embodiment of the disclosure;

FIG. 48B is an image of FIG. 48A generated by the surgical imaging device of FIG. 48 according to an embodiment of the disclosure;

FIG. 48C is a three-dimensional image of aligned elongate side ports for determining the orientation of the primary screw according to an embodiment of the disclosure;

FIG. 48D is an image of FIG. 48C has generated by the surgical imaging device of FIG. 48 according to an embodiment of the disclosure;

FIG. 48E is a sectional view along plane E-E of FIG. 48J;

FIG. 48F is a three-dimensional image of aligned elongate side ports for determining the orientation of the primary screw according to an embodiment of the disclosure;

FIG. 48G is an image of FIG. 48F generated by the surgical imaging device of FIG. 48 according to an embodiment of the disclosure;

FIG. 48H is a three-dimensional image of aligned elongate side ports for determining the orientation of the primary screw according to an embodiment of the disclosure;

FIG. 48I is an image of FIG. 48H generated by the surgical imaging device of FIG. 48 according to an embodiment of the disclosure;

FIG. 48J is a three-dimensional image of aligned elongate side ports for determining the orientation of the primary screw according to an embodiment of the disclosure;

FIG. 48K is an image of FIG. 48J has generated by the surgical imaging device of FIG. 48 according to an embodiment of the disclosure;

FIG. 48L is a sectional view along plane L-L of FIG. 48J;

FIGS. 49-51 are sectional views of the inserter and primary screw configured for the routing of the side screw of FIG. 30 through the inserter and being implanted for the anchoring of the primary screw according to an embodiment of the disclosure;

FIG. 52 is a sectional view of a grafting material being disposed in the inserter sleeve and implanted primary screw according to an embodiment of the disclosure;

FIG. 53 is a sectional view of the plunger assembly of FIGS. 1 and 33 in operation to distribute grafting material for the fusion of the sacroiliac joint according to an embodiment of the disclosure;

FIG. 54 is a sectional view of the primary screw and side screws implanted for fusion of a sacroiliac joint according to an embodiment of the disclosure;

FIG. 55 is a sectional view of the primary screw and side screws of FIG. 54 implanted a sacroiliac joint according to an embodiment of the disclosure;

FIG. 56 is a perspective view of components of a second implant system for fusion of a sacroiliac joint according to an embodiment of the disclosure;

FIG. 57 is a proximal perspective view of a primary screw of the implant system of FIG. 56 according to an embodiment of the disclosure;

FIG. 58 is a distal perspective view of the primary screw of FIGS. 57 and 59 according to an embodiment of the disclosure;

FIG. 59 is a proximal perspective view of a primary screw with an alternative structure for interlocking with an inserter according to an embodiment of the disclosure;

FIG. 60 is an enlarged view of a head portion of the primary screw of FIG. 59 according to an embodiment of the disclosure;

FIGS. 61 through 63 are lateral views of the primary screws of FIGS. 57 and 59 according to an embodiment of the disclosure;

FIG. 64 is a sectional view of the primary screw of FIGS. 57 and 59 at plane LXIV-LXIV of FIG. 61 according to an embodiment of the disclosure;

FIG. 65 is a partial perspective view of a tip portion of the primary screws of FIGS. 57 and 59 according to an embodiment of the disclosure;

FIG. 66 is a sectional view of the primary screw of FIGS. 57 and 59 at plane LXVI-LXVI of FIG. 64 according to an embodiment of the disclosure;

FIG. 67 is an enlarged, partial view of FIG. 66 at a distal end of the primary screw according to an embodiment of the disclosure;

FIG. 68 is an enlarged, partial sectional view of the primary screw of FIGS. 57 and 59 at the tip portion according to an embodiment of the disclosure;

FIGS. 69 through 71 are schematic views depicting the effect of offsetting a distal end port of the primary screw of FIGS. 57 and 59 according to an embodiment of the disclosure;

FIG. 72 is a proximal perspective view of an inserter of the implant system of FIG. 56 according to an embodiment of the disclosure;

FIG. 73 is a sectional view along a inserter axis of the inserter of FIG. 72 according to an embodiment of the disclosure;

FIG. 74 is an enlarged, partial perspective view at a distal end of the inserter of FIG. 72 according to an embodiment of the disclosure;

FIG. 75 is distal perspective view of a primary screw driver of the implant system of FIG. 56 according to an embodiment of the disclosure;

FIG. 76 is a sectional view of the primary screw driver at plane LXXVI-LXXVI of FIG. 75 according to an embodiment of the disclosure;

FIG. 77 is an enlarged, partial perspective view at a distal end of an alternative inserter for mating with the primary screw of FIG. 59 according to an embodiment of the disclosure;

FIG. 78 is a perspective, isolated view of interlocking lobe structures of the inserter of FIG. 77 according to an embodiment of the disclosure;

FIG. 79 is an enlarged, distal perspective view of an interlocking lobe structure of FIG. 78 according to an embodiment of the disclosure;

FIG. 80 is an enlarged, proximal perspective view of an interlocking lobe structure of FIG. 78 according to an embodiment of the disclosure;

FIGS. 81 through 84 are plan views of the interlocking lobe structures of FIG. 78 engaging the primary screw of FIG. 59 according to an embodiment of the disclosure;

FIG. 85 is a cutaway view the inserter, primary screw driver, and primary screw of the implant system of FIG. 56 in assembly according to an embodiment of the disclosure;

FIG. 86 is a sectional view of the assembly at plane LXXXVI-LXXXVI of FIG. 85 according to an embodiment of the disclosure;

FIG. 87 is an enlarged perspective view of a side screw of the implant system of FIG. 56 according to an embodiment of the disclosure;

FIG. 88 is an enlarged, partial sectional view of the side screw of FIG. 77 according to an embodiment of the disclosure;

FIG. 89 is a partial, proximal perspective view of a side screw driver assembly of the implant system of FIG. 56 according to an embodiment of the disclosure;

FIG. 90 is a partial, distal perspective view of a side screw driver assembly of the implant system of FIG. 56 according to an embodiment of the disclosure;

FIG. 91 is an enlarged, partial sectional view of the side screw driver of FIG. 90 in assembly with the side screw of FIG. 87 according to an embodiment of the disclosure;

FIG. 92 is a side view of an alternative side screw for the implant system of FIG. 56 according to an embodiment of the disclosure;

FIG. 93 is an opposing side view of the side screw of FIG. 92 according to an embodiment of the disclosure;

FIG. 94 is a sectional side view of the side screw of FIG. 92 according to an embodiment of the disclosure;

FIG. 95 is an enlarged, partial perspective view of a head portion of the side screw of FIG. 92 according to an embodiment of the disclosure;

FIG. 96 is an enlarged, partial view of FIG. 94 according to an embodiment of the disclosure;

FIG. 97 is a sectional view of the side screw at plane LIXVII-LIXVII of FIG. 92 according to an embodiment of the disclosure;

FIG. 98 is a side view of a side screw driver assembly for use with the side screw of FIG. 92 according to an embodiment of the disclosure;

FIG. 99 is an enlarged, partial perspective view of a distal end of the side screw driver assembly of FIG. 98 according to an embodiment of the disclosure;

FIG. 100 is an enlarged plan view of the distal end of the side screw driver assembly of FIG. 98 according to an embodiment of the disclosure;

FIGS. 101 through 105 are enlarged, partial sectional views of the side screw driver assembly of FIG. 98 in use with the side screw of FIG. 92 according to an embodiment of the disclosure;

FIG. 106 is a partial, distal perspective view of a guide rod of the implant system of FIG. 56 according to an embodiment of the disclosure;

FIG. 107 is a partial elevational view of graduated markings of a guide rod of the implant system of FIG. 56 according to an embodiment of the disclosure;

FIG. 108 is a partial, sectional view of a proximal end of a guide rod of the implant system of FIG. 56 according to an embodiment of the disclosure;

FIG. 109 is a partial, section view of the distal end of the guide rod of FIG. 82 in assembly with the proximal end of the guide rod of FIG. 84 according to an embodiment of the disclosure;

FIG. 110 is a first perspective view of a multifunctional handle of the implant system of FIG. 56 according to an embodiment of the disclosure;

FIG. 111 is a second perspective view of the multifunctional handle of FIG. 110 according to an embodiment of the disclosure;

FIG. 112 is a sectional view of the multifunctional handle of FIG. 111 according to an embodiment of the disclosure;

FIG. 113 is an elevational view of a plunger assembly of the implant system of FIG. 56 according to an embodiment of the disclosure;

FIG. 114 is an enlarged, partial perspective view a distal end of a plunger of the plunger assembly of FIG. 113 according to an embodiment of the disclosure;

FIG. 115 is a partial sectional view of a plunger tube and pallet disk of FIG. 113 in partial assembly according to an embodiment of the disclosure;

FIG. 116 is a partial sectional view of the plunger tube and pallet disk of FIG. 115 in assembly according to an embodiment of the disclosure; and

FIG. 117 is a sectional view of an implanted primary screw and side screws according to an embodiment of the disclosure.

### DETAILED DESCRIPTION

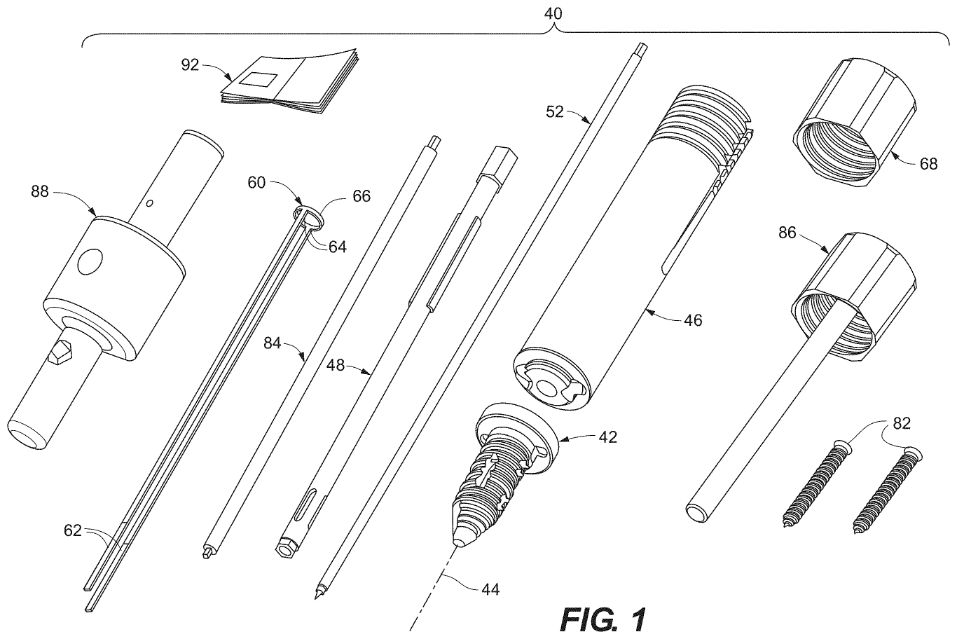

Referring to FIG. 1, an implant system 40 for fusion of a sacroiliac joint is depicted according to an embodiment of the disclosure. The implant system 40 includes a main or primary screw 42 that defines a central axis 44 about which the primary screw 42 rotates, the primary screw 42 being configured to detachably mate with an inserter 46. In some embodiments, a main or primary screw driver 48 is configured to access the primary screw 42 through the inserter 46. The primary screw 42, the inserter 46, and the primary screw driver 48 may be configured for sliding over a guide wire or rod 52. The implant system 40 includes a blade assembly 60 including a pair of flexible, elongate blades 62 having proximal ends 64 that are joined to a ring 66. A drive cap 68 may also be included for deployment of the elongate blades 62. The implant system 40 may include one or more side screws 82 for anchoring the primary screw 42, a side screw driver 84 for setting the side screw(s) 82, a plunger assembly 86 for pushing a biologic agent or other grafting material through the primary screw 42, and a multifunctional handle 88 for manipulation of the screw drivers 46 and 84, drive cap 68, and plunger assembly 86. In some embodiments, some or all of the components of the implant system 40 are provided as a kit 90, and may include operating instructions 92 that are provided on a tangible, non-transitory medium. Additional details, functional descriptions, and methods of use for the various components of the implant system 40 are described below.

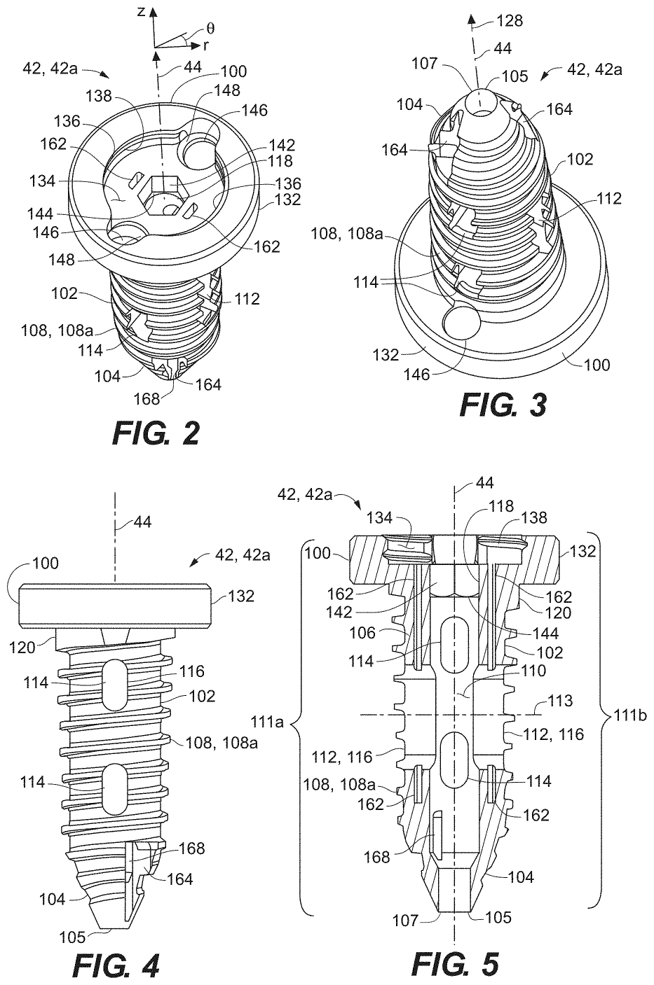

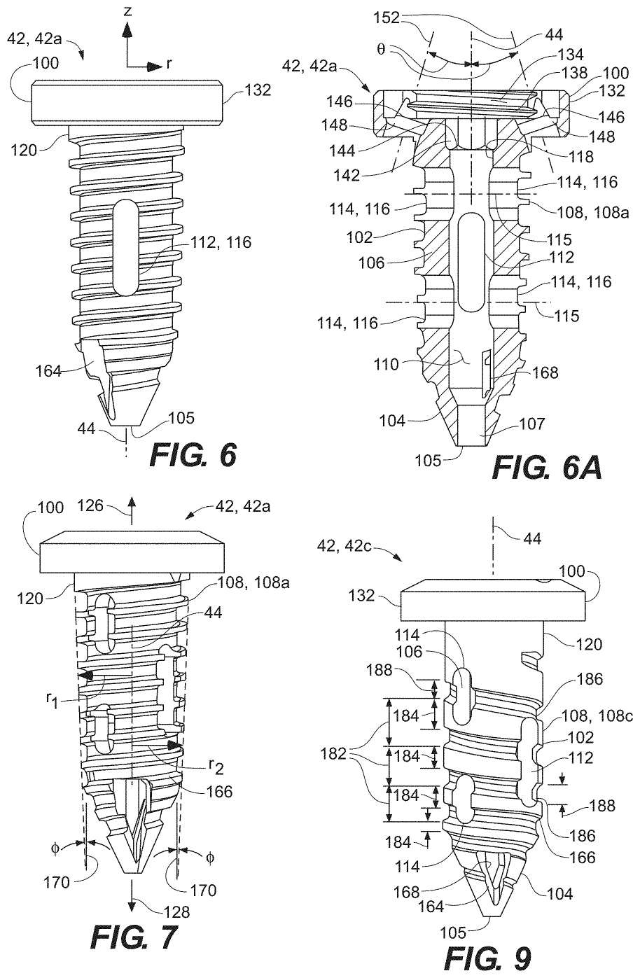

Referring to FIGS. 2 through 7, a primary screw 42a is depicted according to an embodiment of the disclosure. The primary screw 42a includes a head portion 100 and a body portion 102, the body portion 102 including a tip portion 104 having a distal extremity 105, the body portion including a side wall 106 concentric about the central axis 44. The side wall 106 defines an opening 107 at the distal extremity 105 for passage of the guide rod 52. The side wall 106 defines external threads 108a, an interior chamber 110, and at least one elongate side port 112. In the depicted embodiment, there are two such elongate side ports 112 diametrically opposed and about the central axis 44 on opposing lateral sides 111a and 111b of the primary screw 42 and centered about a lateral port axis 113 (FIG. 5). Additionally, diametrically opposed side ports 114 may also be defined that extend through the side wall 106, and centered about respective lateral port axes 115 (FIG. 6A). In some embodiments, the lateral port axes 113, 115 intersect and are perpendicular to the central axis 44 of the primary screw. Also, the lateral port axis 113 may be orthogonal to the lateral port axis or axes 115 when viewed along the central axis 44. Each of the side ports 112, 114 are in fluid communication with the interior chamber 110 and defines a respective external opening 116 that faces exterior to the body portion 102 and extends through the external threads 108. The interior chamber 110 is accessible from an opening 118 defined at a proximal end 120 of the body portion 102.

Herein, primary screws, the associated external threads, and the side screw ports 146 are referred to generically or collectively by reference characters 42 and 108, respectively, with specific primary screws 42 and threads 108 being referred to with a letter suffix (e.g., primary screw 42a having external threads 108a). Also, a “proximal” direction 126 (FIG. 7) of the primary screw 42, as well as the implant system 40 generally, extends parallel to the central axis 44 and toward the operator; a “distal” direction 128 extends opposite the proximal direction 126, i.e., away from the operator.

In some embodiments, the head portion 100 of the primary screw 42 includes a flange 132 that extends radially beyond the side wall 106. The flange 132 at least partially surrounds a recess 134 relative to an exterior proximal face 135 , the recess 134 extending distally to an interior proximal face 137 of the head portion 100. The recess 134 is bounded by one or more inner wall portions 136 of the flange 132. For the primary screw 42a, two such wall portions 136 define a circular arc segment about the central axis 44. In some embodiments, the inner wall portion(s) 136 defines an interior thread 138. The interior thread 138 may be female. In some embodiments, the opening 118 defines a socket 142 that extends distally from the interior proximal face 137 at the base of the recess 134. The socket 142 may be of any suitable shape for torsional coupling with a tool, such as a polygonal shape (triangle, rectangle, square, hexagon, or octagon—hexagonal shape being depicted), a cross, or a hexalobular internal drive feature. In some embodiments, the radial dimension of the interior chamber 110 at the proximal end 120 of the body portion 102 and adjacent the socket 142 is smaller than a maximum radial dimension of the socket 142, thus defining a registration surface 144 at the interface of the socket 142 and the interior chamber 110.

In some embodiments, the flange 132 and proximal end 120 of the body portion 102 defines at least one side screw port 146 for receiving one of the side screws 82. Two such side screw ports 146 are depicted. Each side screw port 146 may extend radially beyond the inner wall portion(s) 136 and may include a countersink seat 148 for registration of the heads of the side screws 82. Each side screw port 146 extends along a side screw port axis 152 that defines an acute angle θ1 relative to the central axis 44. In some embodiments, the side screw port axes 152 are coplanar.

Herein, primary screws, the associated external threads, and the side screw ports are referred to generically or collectively by reference characters 42, 108, and 146 respectively, with specific primary screws 42, external threads 108, and side screw ports 146 being referred to with a letter or decimal suffix (e.g., primary screw 42d; external threads 108e; side screw ports 146g or 146.1).

The body portion 102 may also defines at least one blade passage 162 that extends axially into the side wall 106, the blade passage(s) 162 being accessible from the proximal end 120 of the body portion 102. There are two such blade passages 162 in the depicted embodiment. In some embodiments, the blade passage(s) 162 extend through the thickness of the elongate side port(s) 112 and terminates distal to the elongate side port(s) 112.

The tip portion 104 may include at least one self-tapping structure 164. The depicted embodiment includes two such self-tapping structures 164. In some embodiments, the self-tapping structure(s) 164 define an aperture 168 that is in fluid communication with the interior chamber 110.

The threads 108a of the primary screw 42a define outer radii that gradually diminish along the body portion 102 in the distal direction 128 (FIG. 7), thereby defining thread radii that are greater near the proximal end 120 than near a tip junction 166 at a base of the tip portion 104. Representative outer radii r1 and r2 relative to the central axis 44 are depicted in FIG. 7, with radius r2 being distal to and less than radius r1. The effect is that the thread radii define a tapered angle ϕ relative to a datum 170 that is parallel to the central axis 44. In the depicted embodiment, the tapered angle ϕ is approximately two degrees. In some embodiments, the tapered angle ϕ is in a range of 0.5 degrees to 5 degrees inclusive. (Herein, a range that is said to be “inclusive” includes the stated endpoints of the range as well as all values between the endpoints.)

Functionally, the greater radii threads 108a near the proximal end 120 of the body portion 102 radially penetrate the bone more than the lesser radii threads 108a near the tip junction 166 of the body portion 102. Accordingly, the threads 108a at the tip junction 166 effectively pre-cut the bone for threads 108a at the proximal end 120. The threads 108a provide for easier initial setting and overall easier implantation of the primary screw 42a, while the larger radii threads 108a, by cutting radially deeper into the bone, act to securely fasten the primary screw 42a.

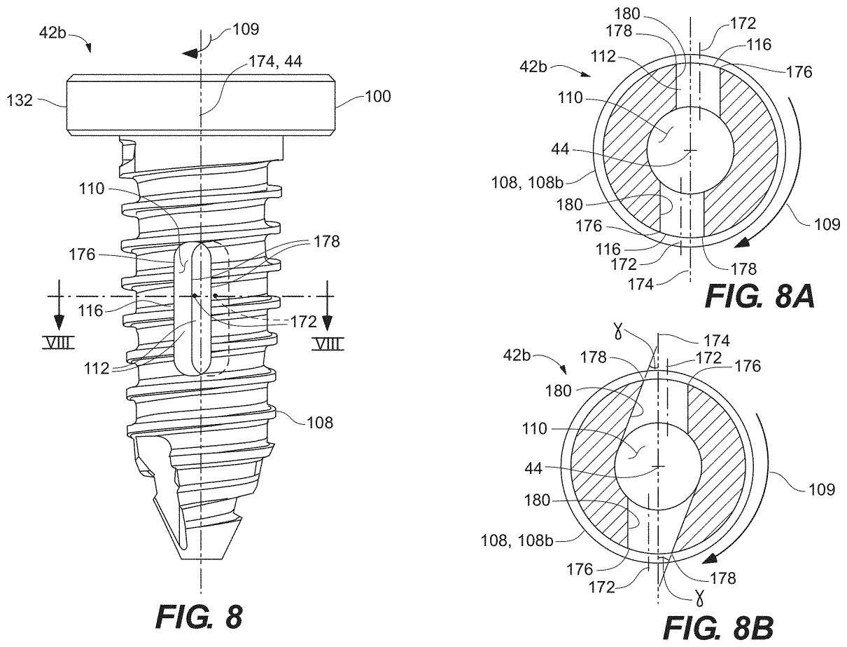

Referring to FIGS. 8, 8A, and 8B, a primary screw 42b with elongate side ports 112 that are laterally offset is depicted according to an embodiment of the disclosure. The primary screw 42b may include many of the same components and attributes as the primary screw 42a, which are identified with same numerical references. The elongate side ports 112 are centered about offset lateral axes 172 that are laterally offset relative the central axis 44, such that the offset lateral axes 172 do not intersect the central axis 44. The primary screw 42b defines a laterally extending mid-plane 174 that extends parallel to the offset lateral axes 172 and is coplanar with the central axis 44. The elongate side ports include a leading tangential edge 176 and a trailing tangential edge 178. The adjectives “leading” and “trailing” refer to the relative positions of the edges 176 and 178 as the primary screw 42 is rotationally threaded into bone in a cutting rotational direction 109. In some embodiments, the leading tangential edge 176 at the external openings 116 of the elongate side ports 112 are closer to the mid-plane 174 than is the trailing edge 178. The elongate side ports 112 include edge walls 180 that terminate at the external openings 116 of elongate side ports 112. In some embodiments, the edge walls 180 extend parallel to the mid-plane 174 (FIG. 8A). In some embodiments, the portion of the edge wall 180 that terminates at the trailing tangential edge 178 defines an acute sweeping angle y relative to the mid-plane 174 (FIG. 8B).

Functionally, the leading tangential edge 176 faces toward the bone as the primary screw 42, 42b is rotated in the cutting rotational direction 109. As such, the trailing tangential edge 178 may act as a cutting edge that scrapes the bone as the primary screw 42, 42b is rotated. By locating the leading tangential edge 176 closer to the mid-plane 174, the trailing tangential edge 178 interfaces with the bone at a more aggressive cutting angle than if the trailing tangential edge 178 were farther from the mid-plane 174, thereby scraping more bone particles which flow into the side ports 112. Embodiments implementing the acute sweeping angle y at the trailing tangential edge 178 may help sweep the bone particles into the interior chamber 110 of the primary screw 42b, thereby mitigating fouling of the side ports 112. The acute sweeping angle γ may also be implemented for side ports 112 that are not offset, for example, the centered side ports 112 of primary screw 42a.

The various primary screws 42 may be coated inside, outside, or both with a bioactive coating to promote growth at the surfaces of the primary screw 42. Examples of bioactive coatings and their implementation are described at Zhang, et al., “Bioactive Coatings for Orthopaedic Implants—Recent Trends in Development of Implant Coatings.” International journal of molecular sciences vol. 15(7) pp. 11878-921, 4 Jul. 2014, doi:10.3390/ijms150711878 (herein “Zhang, et al.”), available at https://www.ncbi.nlm.nih.gov/pmc/articles/PMC4139820/, last visited Feb. 4, 2020, the disclosure of which is hereby incorporated by reference herein in its entirety, except for express definitions contained therein.

Referring to FIG. 9, a primary screw 42c with an alternative thread configuration 108c is depicted according to an embodiment of the disclosure. The primary screw 42c may include many of the same components and attributes as the primary screw 42a, which are identified with same numerical references in FIG. 9. The thread configuration 108c includes both a pitch 182 and a crest 184 that increases along the body portion 102 from the tip junction 166 to the proximal end 120. The thread configuration 108c of FIG. 9 is also characterized as having a thread groove 186 having a substantially constant width 188 along the body portion 102. Primary screws 42 with external threads 108 having an increasing pitch but without increasing crest are also contemplated; such an arrangement can be realized by increasing the width of the thread groove. Primary screws 42 with external threads 108 having increasing crest but without increasing pitch are also contemplated; such an arrangement can also be realized by decreasing width of the thread groove.

Functionally, for configurations that utilize the crest 184 that increases from the tip junction 166 to the proximal end 120 of the body portion 102, the crest 184 effectively forms a wedge in spiral form that pushes bone material axially away from the thread and substantially parallel to the edge wall 106 as the primary screw 42 is rotationally threaded into the bone. The bone material is thereby compressed within the thread groove 186, so that the bone material grips and tightens against the threads 108 of the primary screw 42. For configurations that utilize the pitch 182 that increases from the tip junction 166 to the proximal end 120 of the body portion 102, the external threads 108 near the tip junction 166 effectively interfere with the pathway formed by the external threads 108 near the proximal end 120 in a way that imparts a compressive force on the body portion 102. In this way, the bone material is in a tension against the external threads 108 that compresses the body portion 102 to securely hold the primary screw 42 in place. For the depicted primary screw 42c, having the threads 108c that incorporates both the increasing crest 184 and the increasing pitch 182 from the tip junction 166 to the proximal end 120 of the body portion 102, the combination of the compression of the body portion 102 and the gripping and tightening of the threads 108c within the bone may be realized.

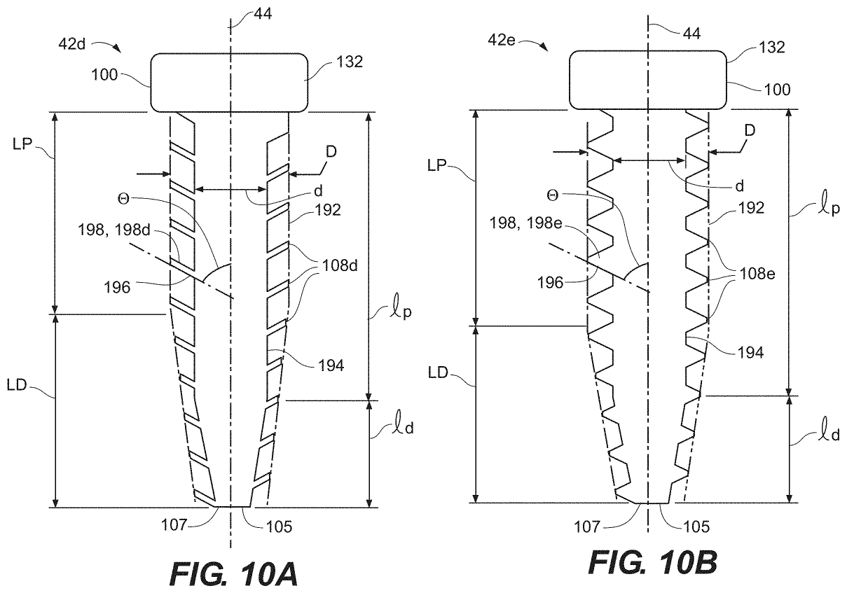

Referring to FIGS. 10A and 10B, primary screws 42d and 42e having swept threads 108d and 108e, respectively, are depicted schematically according to embodiments of the disclosure.

The primary screw 42d includes many of the same components and attributes as the primary screws 42a and 42c, some of which are indicated by same-labeled reference characters. The primary screws 42d and 42e are characterized by threads 108d and 108e having major diameters D that define a crest profile 192 and minor diameters d that define a root profile 194. In some embodiments, the crest profile 192 is substantially parallel to the central axis 44 along a proximal length LP, and tapers toward the central axis 44 along a tapered distal length LD. Similarly, the root profile 194 may be substantially parallel to the central axis 44 along a proximal length lp, and tapers toward the central axis 44 along a tapered distal length ld. The tapered distal lengths LD and ld extend proximally from the distal extremity 105 of the primary screw 42d. In some embodiments, the tapered distal length LD of the crest profile 192 is greater than the tapered distal length ld of the root profile 194. In some embodiments, the tapered distal length LD of the crest profile 192 is in a range 7 millimeters to 13 millimeters inclusive. In some embodiments, the tapered distal length ld of the root profile 192 is in a range 3 millimeters to 7 millimeters inclusive.

In some embodiments, the threads 108d, 108e are inclined distally or “swept back”, so that a distal face 196 the flanks 198 of the threads 108d define an acute swept angle θ2 relative central axis 44. In some embodiments, the acute swept angle θ2 is within a range of 60 degrees to 80 degrees inclusive. In some embodiments, the acute swept angle θ2 is within a range of 75 degrees to 80 degrees inclusive. The flanks 198 of the threads 108d may define a cantilever profile 198c that is canted at the acute swept angle θ2 (FIG. 10A). In other embodiments, the flanks 198 of the threads 108d may define a triangular- or frustum-shaped profile 198d (FIG. 10B).

Functionally, the shorter tapered distal length ld of the root profile 194 relative to the tapered distal length LD of the crest profile 192 promotes pushing of soft tissue such as flesh and muscle radially away from an access approach rather than cutting or tearing the soft tissue. By favoring pushing the soft tissue aside over tearing or cutting, the soft tissue may heal faster. The swept threads 108d also favors the radial displacement of soft tissue instead of cutting or tearing of the tissue. In some embodiments, the displacement of soft tissue eliminates the need for a dilator during the surgical process.

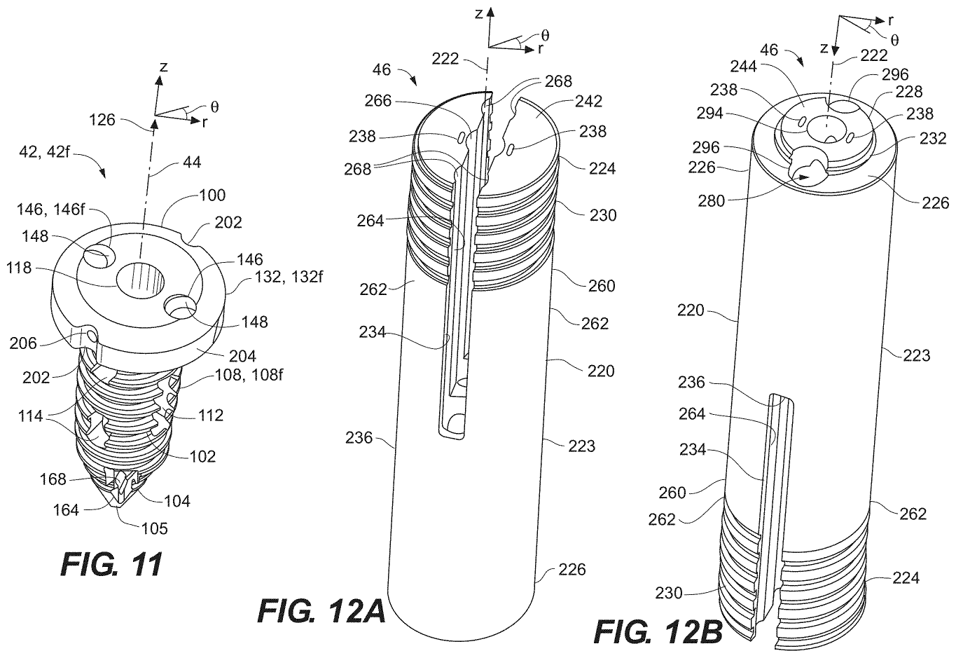

Referring to FIG. 11, a primary screw 42f with a flange 132c is depicted according to an embodiment of the disclosure. The primary screw 42f may include many of the same components and attributes as the primary screws 42a and 42c, some of which are indicated by same-labeled reference characters. The flange 132c is configured for coupling to a tool (not depicted) with notches 202 on a radial outward face 204 thereof. Accordingly, in some embodiments, the head portion 100 need not define the recess 134 of primary screws 42a or 42c. In FIG. 11, there are a pair of diametrically opposed notches 202 rotationally offset from the side screw ports 146. Additional notches are also contemplated, for example three notches spaced at 120 degrees apart or four notches spaced at 90 degrees apart. The flange 132c may include radially extending apertures 206 disposed radially inward at the notches 202.

Functionally, the notches 202 provide an interface for gripping the primary screw 42f with a tool. The apertures 206 may also be part of the tool interface, for alignment, structural enhancement, or both. The tool may be stout enough to enable driving of the primary screw 42f into bone using just the notches 202 or the notches 202 and apertures 206 in combination, so that no additional driving feature, such as the socket 142 of primary screw 42a or 42c, is needed. Alternatively, the tool may be an inserter similar to inserter 46 modified to couple with the notches 202, not designed to drive the primary screw 42f but through which driving tools access the primary screw 42f; in such an arrangement, the head portion 100 may define, for example, structure similar to the socket 142 (not depicted in FIG. 11) for coupling with the driving tools.

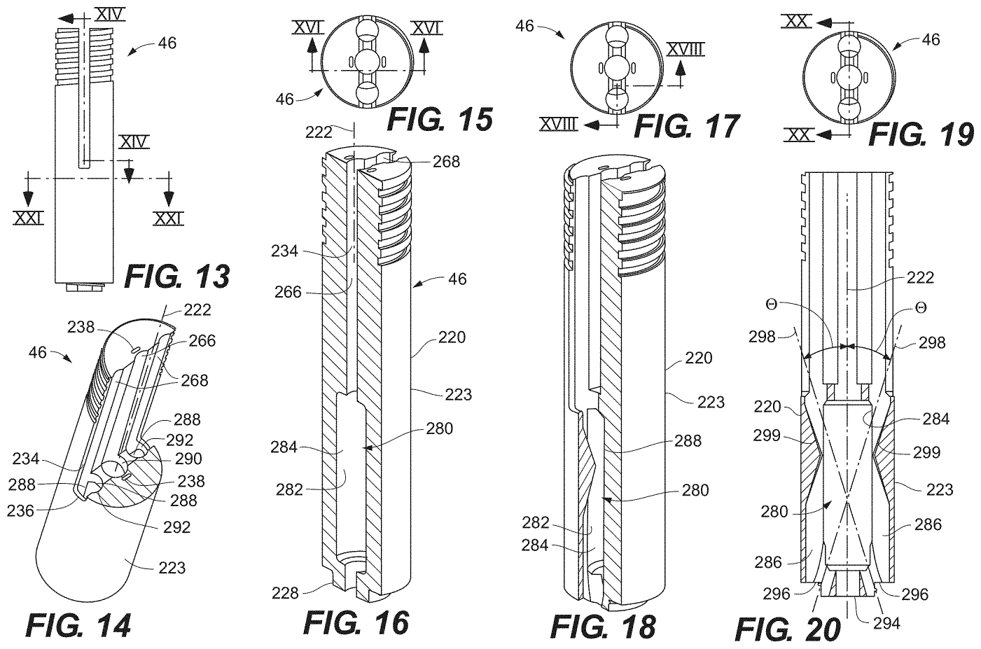

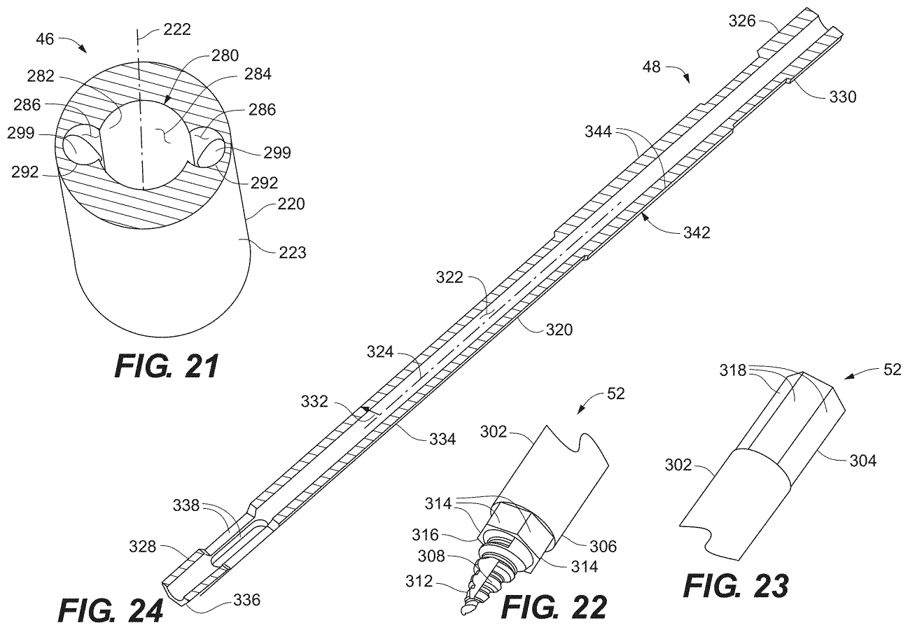

Referring to FIGS. 12A through 22, the inserter 46 is depicted according to an embodiment of the disclosure. The inserter 46 includes a main cylinder 220 concentric about an inserter axis 222 and having an exterior surface 223, a proximal end 224 and a distal end 226, with a boss 228 extending from the distal end 226. The main cylinder 220 may include external threads 230 formed at the proximal end 224. The boss 228 may include an exterior thread 232 configured to threadably engage the interior thread 138 of the inner wall portion(s) 136 of the recess of the primary screw 42a or 42c. In some embodiments, an access slot 234 extends axially from the proximal end 224 of the main cylinder 220, a distal end 236 of the access slot 234 extending to a mid-portion of the main cylinder 220 and passing laterally through the main cylinder 220. The main cylinder defines at least one blade passage 238 that extends axially and parallel to the inserter axis 222, each blade passage 238 passing through a proximal face 242 of the main cylinder 220 and a distal face 244 of the inserter 46.

In some embodiments, the access slot 234 bifurcates a proximal portion 260 of the main cylinder 220 into two opposed arcuate wall portions 262, each including an inner surface 264. Each inner surface 264 defines a central arcuate channel 266 and at least one side arcuate channel 268. The arcuate channels 266 and 268 extend parallel to the inserter axis 222. The central arcuate channels 266 of the opposed inner surfaces 264 are mirrored about the access slot 234 and concentric about the inserter axis 222. The at least one side arcuate channel 268 of the opposed inner surfaces 264 are mirrored about the access slot 234. In the depicted embodiment, each inner surface 264 defines two such side arcuate channels 268 that are on laterally opposing sides of the central arcuate channel 266.

The main cylinder 220 defines an interior chamber 280 having an interior wall 282. In some embodiments, the interior chamber includes a main or central chamber 284 and at least one antechamber 286 that are in fluid communication. In the depicted embodiment, there are two antechambers 286, each coplanar with and distal to the access slot 234. Each antechamber 286 intersects with the central chamber 284, defining a passageway 288 therebetween. The main cylinder 220 defines a central entrance port 290 and at least one side entrance port 292 that are in fluid communication with the interior chamber 280. In the depicted embodiment, the central port 290 is concentric with the inserter axis 222 and provides access to the central chamber 284, and there are two side entrance ports 292, each being defined at the junction of the access slot 234 and a respective one of the antechambers 286. The entrance ports 290, 292 are internal to the main cylinder 220 and located proximate the distal end 236 of the access slot 234.

The main cylinder 220 also defines a central egress port 294 and at least one side egress port 296 that are in fluid communication with the interior chamber 280. The egress ports 294, 296 pass through the distal end 226 and the boss 228 of the inserter 46. Each side entrance port 292 and side egress port 296 combines with the interior chamber 280 to define a cross passage 297 that extends along a respective canted axis 298. In some embodiments, each canted axis 298 crosses the inserter axis 222 and defines the acute angle θ1 relative to the inserter axis 222. In the depicted embodiment, the central egress port 294 is concentric about the inserter axis 222 at the distal end of the central chamber 284. As there are two side entrance ports 292 in the depicted embodiment, there are also cross passages 297 and two side egress ports 296 aligned along two canted axes 298. In the depicted embodiment, the canted axes 298 are coplanar with a central plane of the access slot 234.

The interior wall 282 defines at least one guide ramp 299 that extends radially inward, one for each side entrance port 292. Each guide ramp 299 is centered distal to the corresponding side entrance port 292 and extends adjacent the corresponding canted axis 298. The depicted embodiment, having two side entrance ports 292, also has two guide ramps 299. Also in the depicted embodiment, the guide rams 299 are disposed in the antechambers 286.

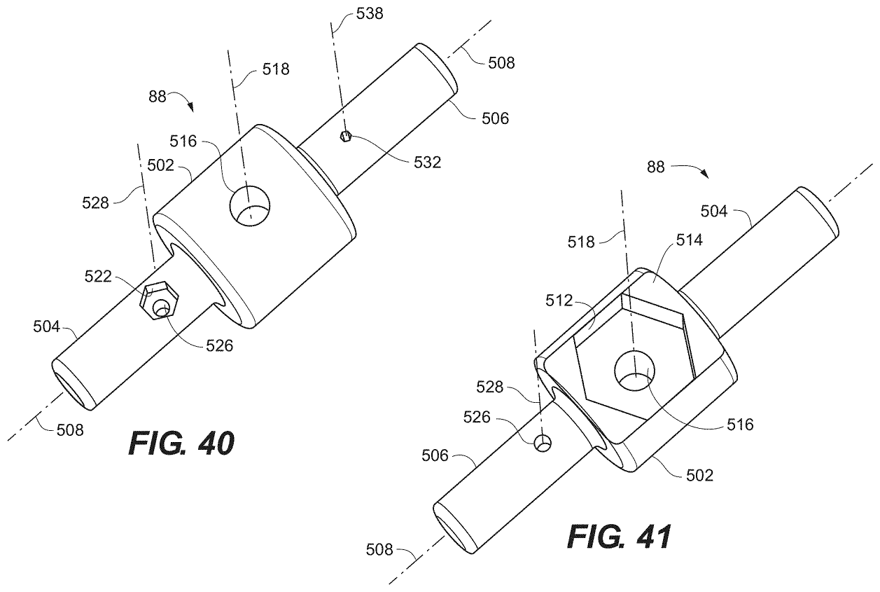

Referring to FIGS. 22 and 23, the guide rod 52 is described in further detail according to an embodiment of the disclosure. The guide rod 52 includes a shaft portion 302 having proximal end 304 and a distal end 306. The distal end 306 may include a self-tapping threaded structure 308 at a distal extremity 312. In some embodiments, flats 314 are formed adjacent the threaded structure 308 at the distal end 306, forming a driving head 316 defining a polygonal cross section that is shaped and dimensioned to mate with a socket 420 (FIG. 29) of the side screw(s) 82. In some embodiments, the driving head 316 is dimensioned to form a press fit with the socket 420, to provide a stable coupling between the driving head 316 and the side screw 82. Flats 318 may also be formed at the proximal end 304, for mating with a socket 532 on the multifunctional handle 88 (FIG. 40).

Functionally, the self-tapping threaded structure 308 enables the guide rod 52 to be readily anchored at a penetration site where the primary screw 42 is to be implanted. In some embodiments, the flats 314 at the distal end 306, being configured to mate with the socket 420 of the side screw(s) 82, enable the guide rod 52 to also serve as a driver for the side screw(s) 82. Of course, the sockets 420 of such side screws require sufficient depth to accommodate the threaded structure 308 when the distal end 306 is inserted into the socket 420. The flats 318 at the proximal end 304 enable torsional driving of the guide rod 52, be it for anchoring the threaded structure 308 into bone or for driving the side screws 82.

Referring to FIG. 24, the primary screw driver 48 is described in more detail according to an embodiment of the disclosure. The primary screw driver 48 includes a shaft portion 320 defining a central passage 322 concentric about a central axis 324. The central passage 322 passes through the entire length of the shaft portion 320, from a proximal end 326 and a distal end 328. The proximal end includes wrench flats 330 formed thereon. In some embodiments, the wrench flats 330 extend radially beyond a nominal radius 332 of a main body 334 of the shaft portion 320 (depicted). Alternatively, the wrench flats 330 may be radially inset from the main body 334. The wrench flats 330 may define a polygonal shape, such as a triangle, square, hexagon (depicted), or octagon. The distal end 328 includes a driving head 336 shaped for mating with the socket 142 of the primary screw 42. The driving head 336 may be radially inset from the nominal radius 332 of the main body 334 of the shaft portion 320. The main body 334 may also define one or more lateral through-holes 338 that are in fluid communication with the central passage 322. In the depicted embodiment, there are four such lateral through-holes 338 located near the distal end 328, the through-holes 338 being axially elongate and uniformly distributed about the central axis 324.

In some embodiments, the primary screw driver 48 includes a ring guide 342 for alignment and rotational coupling with the ring 66 of the blade assembly 60. The ring guide 342 may include a pad or rail 344 mounted to or formed on the main body 334 of the shaft portion 320 that extends beyond the nominal radius 332 of the main body 334 of the shaft portion 320. In the depicted embodiment, there are two such rails 344, diametrically opposed and extending axially along the main body 334 near the proximal end 326. More or less than two rails 344 are contemplated. Alternatively, the ring guide 342 may be of other forms, including flats or grooves that extend distally from the proximal end 326 of the screw driver 48 and are inset from the nominal radius 332 of the main body 334 of the shaft portion 320.

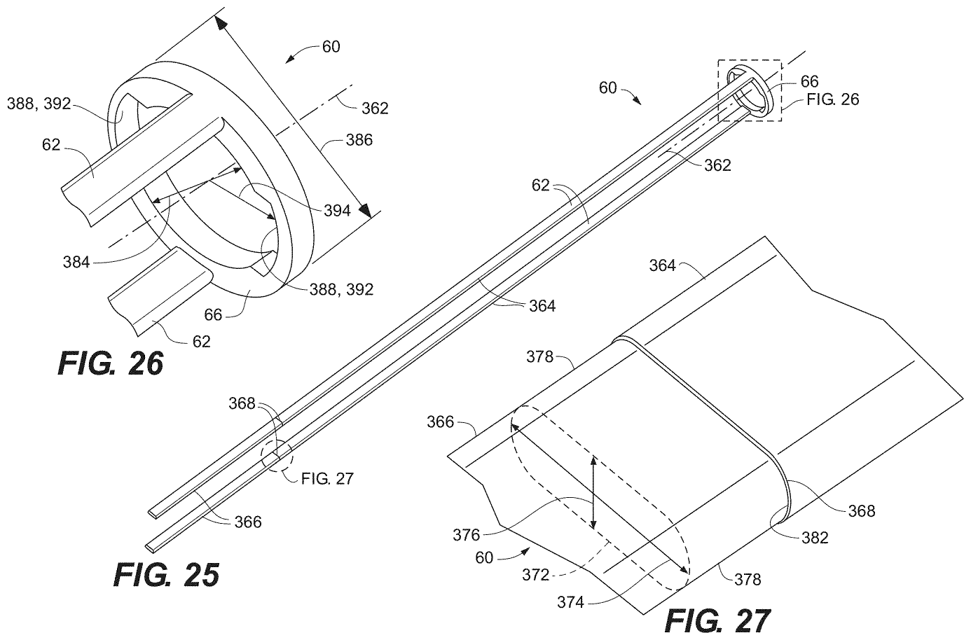

Referring to FIGS. 25 through 27, the blade assembly 60 is described in more detail according to an embodiment of the disclosure. In some embodiments, the blade assembly 60 is axisymmetric about a ring axis 362 that is concentric with the ring 66. In some embodiments, each flexible, elongate blade 62 includes a proximal portion 364 and a distal portion 366 that transition together at a junction 368. In some embodiments, the distal portion 366 defines an oblong cross-section 372 having a major dimension 374 and a minor dimension 376, the major dimension 374 extending tangential to the ring axis 362 and the minor dimension 376 extending substantially radially relative to the ring axis 362. The distal portion 366 defines edges 378 at the extremities of the major dimension 374. The edges 378 may be of any appropriate geometry for tissue cutting, including a radiused edge (depicted), a centered ridge, or an offset ridge.

The proximal portion 364 is of greater dimensions than the distal portion 366. In the depicted embodiment, the proximal portion 364 defines a similarly shaped but enlarged cross-section. Other cross-sections may be utilized for the proximal portion 364, including a square, rectangular, circular, or elliptical cross-section. The junction 368 may define a step transition 382 (depicted) or a tapered transition.

The ring 66 defines an inner radius 384 and an outer radius 386, and includes features 388 for sliding engagement with the ring guide 342. In some embodiments, the features 388 include at least one keyway 392 defined in the ring 66, the keyway 392 extending radially outward from the inner radius 384 to define a maximum inner radius 394 of the ring 66. For the depicted embodiment, there are two such keyways 392, each sized and shaped to slide over the rails 344 of the primary screw driver 48.

Functionally, the larger cross-section of the proximal portion 364 provides stoutness to prevent buckling of the proximal portion 364 when the distal portion 366 of the blades 62 are axially compressed to flex the distal portion 366. The oblong cross-section 372 of the distal portion 366, having the major dimension 374 extending tangentially, provides stiffness in the tangential direction so that the distal portion 366 of the blade 62 flexes in the radial direction.

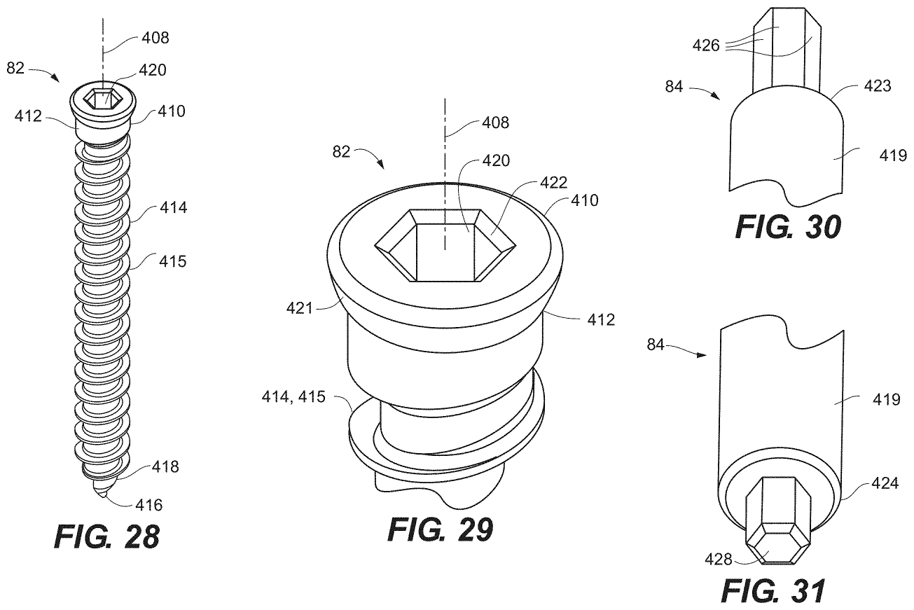

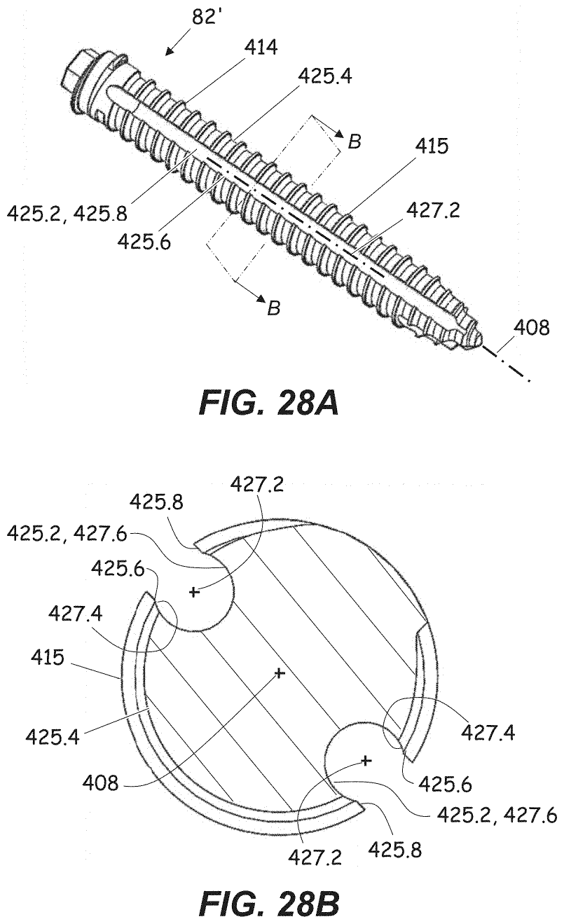

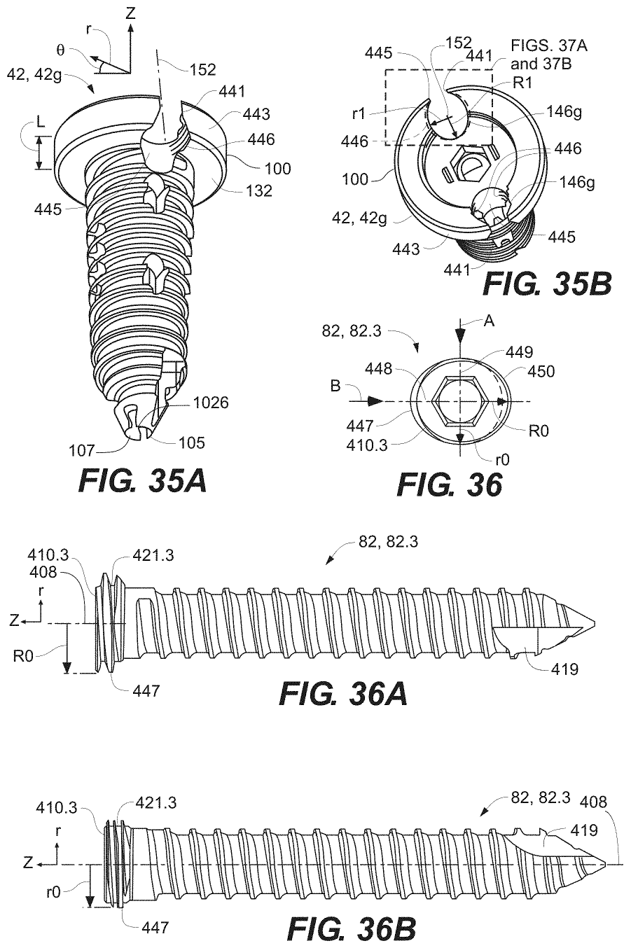

Referring to FIGS. 28 and 29, the side screw(s) 82 are described in further detail according to an embodiment of the disclosure. Each side screw 82 defines a side screw axis 408 and includes a head 410 at a proximal end 412 thereof, from which a threaded shaft 414 depends. The threaded shaft 414 includes external threads 415 may converge to a point 416 at a distal end 418. In some embodiments, the side screws 82 include self-tapping flutes 419 (e.g., FIG. 36A). The head 410 defines a socket 420 and may include a flange 421 that extends radially beyond the threaded shaft 414. In some embodiments, the socket 420 includes a chamfered lead-in 422. The socket 416 may define any one of a variety of shapes, such as a triangle, rectangle, square, hexagon (depicted), octagon, cross, hexalobular internal drive feature, or other shapes suitable for torsional driving of the side screw 82.

Referring to FIGS. 28A and 28B, a side screw 82′ is depicted according to an embodiment of the disclosure. The side screw 82′ includes some of the same components and attributes as the side screw 82 of FIG. 28, some of which are identified with same-numbered reference characters. A distinction of the side screw 82′ is one or more side cavities 425.2, each breaching a root 425.4 of the threads 415 of the threaded shaft 414 to define a respective side opening or window 425.6. In some embodiments, a channel 425.8 extends axially through the threads 415 adjacent each side window 425.6. Each side cavity 425.2 may define a side cavity axis 427.2 that extends axially along the shaft threaded shaft 414 and is offset from the side screw axis 408. Accordingly, the each side cavity axis 427.2 is disposed between the respective side window 425.6 and the side screw axis 408. In some embodiments, the side cavity or cavities 425.2 do not encompass or encroach the side screw axis 408 (depicted). The side window(s) 425.6 may define a sharp edge 427.4 at the root 425.4 of the external threads 415. Each channel 425.8 eliminates the portions of the external threads 415 that would otherwise bridge or extend partially over the respective side window 425.6 while providing a cutting angle at the sharp edge 427.4 the side window(s) 425.6 along the root 425.4.

Functionally, the one or more side cavities 425.2 provide structure into which bone tissue can grow to help secure the side screw 82′ over time. In some embodiments, the side cavities 425.2 collect bone fragments as the side screw 82′ is threaded into the bone, thereby providing material to promote the growth of the bone tissue into the side cavities 425.2. The sharp edges 427.4 of the side window 425.6 may function to cut and shave bone material and rake the bone material into the side cavities 425.2 as the side screw 82′ is threaded into the bone. The side cavities 425.2 of FIG. 28B are depicted as defining cross-sections 427.6 that are substantially constant radius (circular); other cross-sections are contemplated, including U-shaped, V-shaped, and an open rectangular shaped channel cross-sections. An example of a “V-shaped” cross-section for the side cavities is depicted at FIG. 97 and discussed attendant thereto. An “open rectangular” shape is one that defines three sides of a rectangle, with the fourth side being open to define the side window 425.6.

In some embodiments, supplemental bone growth materials such as biologics are packed into the side cavities 425.2 prior to setting the side screw 82′ into the bone. In some embodiments, the side cavities 425.2 are treated with a bioactive coating to promote growth of bone tissue into the side cavities 425.2. The side screws 82′ may also be treated more generally with the bioactive coating to promote growth of bone tissue between the external threads 415. Examples of bioactive coatings and their implementation are described at Zhang, et al., incorporated by reference herein above.

The exposure of the captured bone material, biologic packing, and/or bioactive coating through the side window 425.6 also promotes bone growth axially along the bone screw 20 and across interfaces of bone joints. For example, in applications where the bone screws 20 are utilized for sacroiliac joint fusion, bone tissue growth may occur axially along the side window 425.6 and channels 425.8 to bridge the cartilage between the sacrum and the ilium, which promotes the stability and robustness of the fusion.

Referring to FIGS. 30 and 31, the optional side screw driver 84 is described in further detail according to an embodiment of the disclosure. The side screw driver 84 includes a shaft 419 proximal end 423 and distal end 424. The proximal end 423 may include flats 426 for application of a wrench (e.g., multifunctional handle 88) for torqueing the side screw driver 84. In some embodiments, the flats 426 define a polygonal cross-section (hexagonal depicted). The distal end 424 may include a driving head 428. The driving head 424 is configured to mate with the socket 420 of the side screw(s) 82. Accordingly, for the depicted embodiment, the driving head 428 of the side screw driver 84 is hexagonal. Like the driving head 316 of the guide rod 52, the driving head 428 may be dimensioned to form a press fit with the socket 420, to provide a stable coupling between the driving head 428 and the socket 420 while guiding the side screw 82 through the inserter 46 (described below).

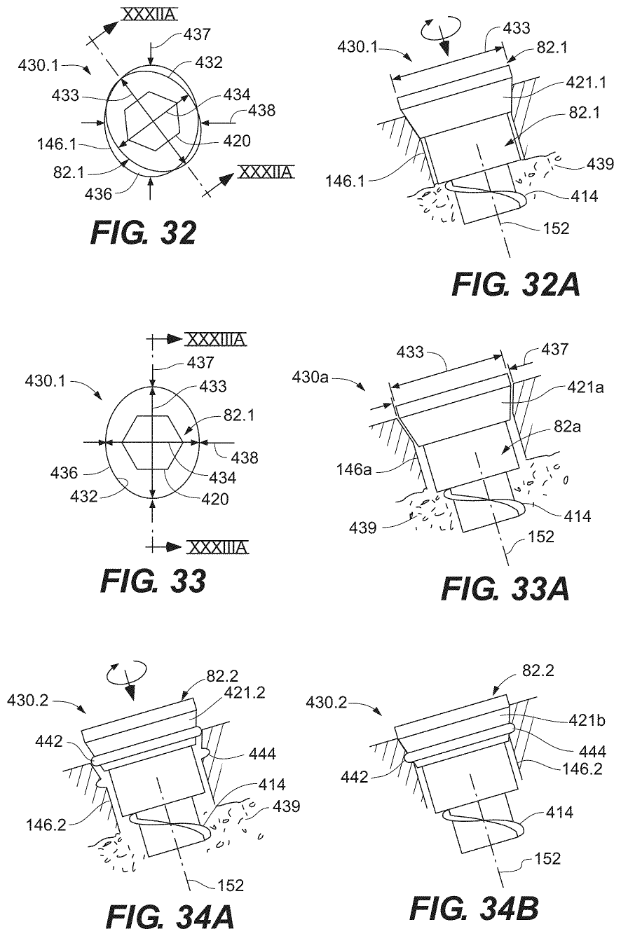

Referring to FIGS. 32 through 33A, a locking configuration 430.1 for a side screw 82.1 is depicted according to an embodiment of the disclosure. In this embodiment, a flange 421.1 of the side screw 82.1 defines an oblong or elliptical shape 432 characterized by a major dimension 433 and a minor dimension 434. Side screw ports 146.1 of the primary screw 42 may also define an oblong or elliptical shape 436 characterized by a major dimension 437 and a minor dimension 438. The oblong shape 436 of side screw ports 146.1 are dimensioned to fully receive and mate with the oblong shape 432 of the flange 421.1 when the major axes 433 and 437 are aligned, as in FIG. 33.

In operation, the side screw 82.1 is screwed into a bone 439 and the flange 421.1 brought into initial contact with the edges of the side screw port 146.1. For this initial contact, the respective major axes 433 and 437 of the oblong shapes 432 and 436 are not aligned (FIG. 32) and the side screw 82.1 is not fully set within the screw port 146.1 (FIG. 32A). The surgeon continues to drive the screw 82.1 into the bone 439, causing the major dimension 433 of the flange 421.1 to rotate toward alignment with the major dimension 437 of the side screw port 146.1, and also causing the side screw 82.1 to be drawn against and into the side screw port 146.1. The side screw 82.1 is thus driven into the bone 439 until the major axes 433 and 437 are aligned and the flange 421.1 is seated within the side screw port 146.1 (FIGS. 33 and 33A). The mating of the oblong shapes 432 and 436 resists rotation of the side screws 82.1, thereby locking the side screws 82.1 in place and inhibiting the side screws 82.1 from rotating after implantation and backing out of the side screw sockets 146.1.

Referring to FIGS. 34A and 34B, a second locking configuration 430.2 for a side screw 82.2 is depicted according to an embodiment of the disclosure. In this embodiment, a flange 421.2 of the side screw 82.2 includes a detent ring 442, and a side screw port 146.2 includes a complementary groove 444 configured to receive the detent ring 442. The detent ring 442 projects radially outward from the contour of the flange 421.2. Alternatively, the screw port may define the detent, and the flange may define the complementary groove (not depicted).

In operation, the side screw 82.2 is screwed into the bone 439 and the detent ring 442 brought into initial contact with the side screw port 146.2 (FIG. 34A). For this initial contact, the detent ring 442 is not disposed within the groove 444. The surgeon continues to drive the screw 82.2 into the bone 439, causing the detent ring 442 to be drawn against the side screw port 146.2 and toward the groove 444 until the detent ring 442 snaps into the groove 444 (FIG. 34B). The mating of the detent ring 442 and groove 444 prevents the side screws 82.2 from backing out of the side screw ports 146.2.

The side screws 82.1, 82.2 and side ports 146.1, 146.2, have many of the same components and attributes as the side screws 82 and side screw ports 146, some of which are indicated in FIGS. 32 through 33A by same-labeled reference characters. It is understood that discussion herein pertaining to the side screws 82 and side screw ports 146 apply generally to embodiments utilizing the side screws 82.1, 82.2 and the side ports 146.1, 146.2.

Referring to FIGS. 35A through 37B, a third locking configuration 430.3 and components thereof is depicted according to an embodiment of the disclosure. A side screw 82.3 and a primary screw 42g of the third locking configuration 430.3 may include many of the same components and attributes as the side screws 82 and primary screws 42 generally, some of which are indicated by same-labeled reference characters. For the third locking configuration 430c, the primary screw 42g may be configured so that side screw ports 146g define a breach or gap 441 that extends an axial length L of an outer diameter surface 443 of the flange 132, so that the side screw ports 146g do not form a closed diameter hole. Rather, the side screw ports 146g may instead define what may be characterized as a “C-shape” when viewed along the respective side screw port axis 152 (FIG. 35B). In some embodiments, the side screw port 146g defines an oblong through hole having a major radius R1 and a minor radius r1 about the side screw port axis 152. In some embodiments, a socket wall 445 of each side screw port 146g defines female threads 446 having a constant radius (circular) root diameter about the side screw port axis 152, the root diameter defining a maximum radius R.

The side screw 82.3 defines a head 410.3 having oblong threads 447. The oblong threads 447 extend radially from a flange 421.3 of the head 410.3 along a major axis 448 to define a major radius R0 (FIGS. 36, 36A). The radial dimension of the oblong threads 447 taper off in the tangential direction to define a minor axis 449 that is orthogonal to the major axis 448, the oblong threads 447 defining a minor radius r0 along the minor axis 449 (FIGS. 36, 36A). The oblong threads 447 thereby define an oblong profile 450 of the side screw 82.3 when viewed from the top end (FIG. 36), wherein the threads at the major radius R0 have a substantially greater radial engagement depth with the female threads 446 than do the threads at the minor radius.

In some embodiments, the female threads 446 are circular about the side screw port axis 152. Because the radius of the circular female threads 446 are less than the major radius R1 of the side screw port 146g but greater than the minor radius r1 of the side screw port 146g, the circular female threads 446 may cut into only a portion of the socket wall 445 centered about the minor radius r1 (depicted). In some embodiments, the pitch of the threads 446 and 447 are the same pitch as the threads of the side screw 82.3.

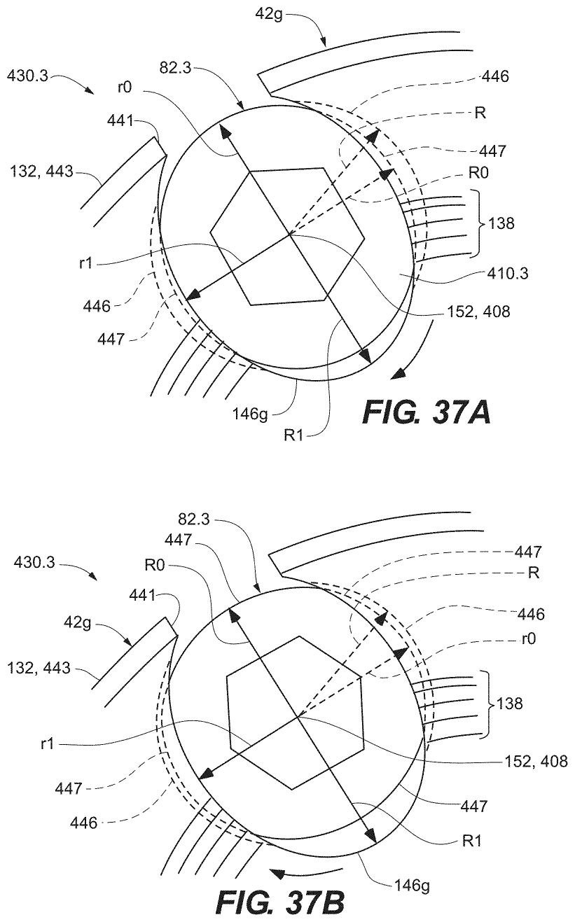

In operation, the side screw 82.3 is screwed into a bone and the oblong threads 447 of the side screw 82.3 brought into engagement with the side screw port 146g of the primary screw 42g. Because of the though hole of the side screw port 146g is oblong, there is more contact surface between the female threads 446 and the oblong threads 447 as the major radius R0 of the oblong threads 447 is rotated within the circular female threads 446 into alignment with the major radius R1 of the side screw port 146g (FIG. 37A, depicting the major radius R0 of the oblong threads 447 in phantom lodged within the female threads 446), the resistance to turning the side screw 82.3 within the female threads 446 decreases. As the major radius R0 of the oblong threads 447 are rotated out of alignment with the major radius R1 of the side screw port 146g and into alignment with the minor radius r1 of the side screw port 146g, the resistance to turning the side screw 82.3 within the female threads 446 decreases because there is diminishing overlap between the oblong threads 447 and the circular female threads 446. There is minimum overlap between the oblong threads 447 and the circular female threads 446 when the major radius R0 of the oblong threads is aligned with the major radius R1 of the side screw port 146g (FIG. 37B).

Functionally, the oblong threads 447 maintain threaded engagement with the female threads 446 of the side screw port 146g as the side screw 82.3 is driven into the bone, thereby establishing a stable axial relationship between the side screw 82.3 and the primary screw 42g. For embodiments where the pitch of the threads 446 and 447 are the same pitch as the threads of the side screw 82c, the axial tension that the side screw 82.3 exerts on the bone is reduced relative to embodiments where the pitch of the threads 446 and 447 are different from the pitch of the threads of the side screw 82c, so that the side screw 82.3 can be backed out and then retightened without exerting additional fatigue on the bone due to mismatched thread pitches.

Upon being driven into place, the side screw 82.3 is oriented so that the major radius R0 is aligned with the major radius R1 of the side screw port 146g, with the minor radius r0 of the oblong threads 447 being aligned with the minor radius r1 of the side screw port 146g and extending into and engaging the circular female threads 446. In this orientation, the minor radius r0 of the oblong threads 447 are engaged and centered within the threads 446 of the side screw port 146g at a position of minimum overlap (FIG. 37B). Because the overlap is minimum, the side screw 82.3 is in a state of equipoise when in the position illustrated in FIG. 37B. That is, in the position illustrated in FIG. 37B, the rotational forces on the side screw 82.3 are substantially balanced so that there is no motivation for the side screw 82.3 to rotate. Accordingly, while a surgeon may readily rotate the side screw 82.3 out of equipoise during implantation, the forces encountered by the side screw 82.3 after final implantation (e.g., vibration, flexing) are not enough to rotate the side screw 82.3 out of equipoise. As such, the side screw 82.3 will tend to remain in the equipoise position after implantation, so that the side screw 82.3 is effectively secured rotationally in the implanted equipoise orientation of FIG. 37B. In some embodiments, the torsional resistance to rotating the side screw 82.3 out of equipoise is in a range of 1.5 inch-pounds to 4 inch-pounds inclusive. In some embodiments, the torsional resistance to rotating out of equipoise is in a range of 2 inch-pounds to 3 inch-pounds inclusive.

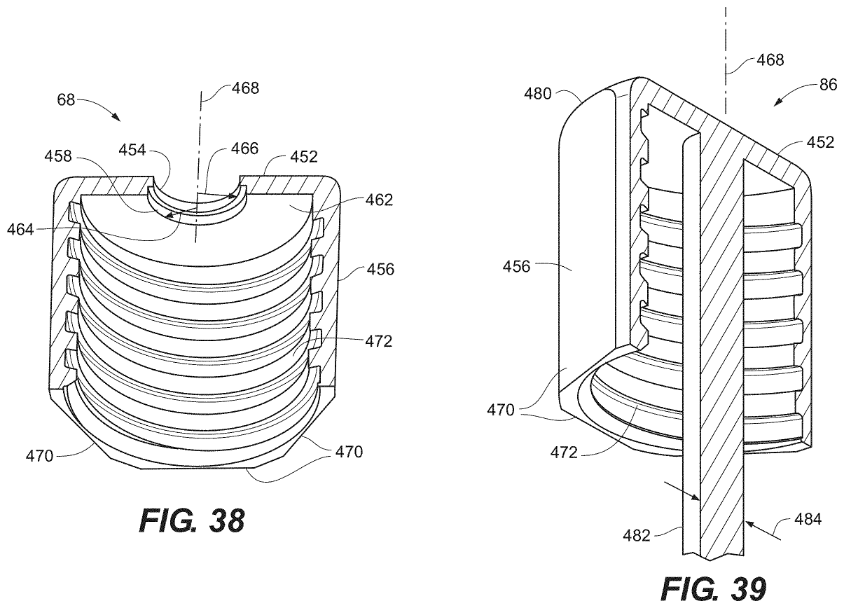

Referring to FIG. 38, an enlarged sectional view of the drive cap 68 is described in further detail according to an embodiment of the disclosure. The drive cap 68 includes an end portion 452 defining a through-aperture 454 and from which a skirt portion 456 depends. An annular recess 458 is defined on an interior surface 462 of the end portion 452, the annular recess 458 defining an outer radius 464 and being open to an inner radius 466 of the through-aperture 454. The end portion 452, through-aperture 454, skirt portion 456, and annular recess 458 are concentric about a drive axis 468 of the drive cap 68. The exterior of the skirt portion 456 may define a plurality of flats 470, the flats 470 defining a polygonal cross-section such as a triangle, square, hexagon (depicted), or octagon.

The skirt portion 456 may define internal threads 472 configured to threadably engage the external threads 230 of the inserter 46. The inner radius 466 is dimensioned to enable the primary screw driver 48 to pass therethrough, including any ring guide 342 that may extend beyond the nominal radius 332 of the main body 334 of the shaft portion 320 of the primary screw driver 48. Accordingly, in such embodiments, the inner radius 466 is at least the maximum inner radius 394 of the keyway 392 defined in the ring 66. The annular recess 458 is configured to receive the ring 66 with a fit that enables the ring 66 to rotate within the annular recess 458. That is, the outer radius 464 of the annular recess 458 is dimensioned to enable the ring 66 to slidably rotate within the annular recess 458 when the ring 66 is seated within the annular recess 458.

Referring to FIG. 39, a sectional view of the plunger assembly 86 is described in further detail according to an embodiment of the disclosure. The plunger assembly 86 includes a cap 480 that may have many of the same components and attributes as the drive cap 68, some of which are indicated by same-labeled reference characters. A plunger stem 482 defines an outer diameter 484 and depends from the end portion 452, extending axially beyond the skirt portion 456.

Referring to FIGS. 40 and 41, perspective views of the multifunctional handle 88 is described in further detail according to an embodiment of the disclosure. In some embodiments, the multifunctional handle 88 includes a body portion 502 that separates opposed first and second handle portions 504 and 506, the body portion 502 and opposed handle portions 504 and 506 being arranged along a lateral axis 508. The body portion 502 defines a socket 512 accessible from a first side 514 of the body portion 502 and a through-aperture 516 that extends from the socket 512 through the body portion 502. The socket 512 and through-aperture 516 are concentric about a central handle axis 518, the central handle axis 518 being perpendicular to the lateral axis 508. The first handle portion 504 may also define a socket 522 and through-aperture 526 that extends from the socket 522 through the first handle portion 504, the socket 522 and through aperture 526 being concentric about a first handle axis 528 that is perpendicular to the lateral axis 508. In some embodiments, the second handle portion 506 also defines a socket 532 that is concentric about a second handle axis 538, the second handle axis 538 being perpendicular to the lateral axis 508.

The socket 512 of the body portion 502 is configured for detachable coupling with the flats 470 of the drive cap 68 and the cap 480 of the plunger assembly 86. Accordingly, in the depicted embodiment, the socket 512 defines the hexagonal shape of the drive cap 68 and the cap 480 of the plunger assembly 86. The socket 522 of the first handle portion 504 is configured for detachable coupling with the wrench flats 330 of the primary screw driver 48. Accordingly, for the depiction of multifunctional handle 88, the socket 522 defines the hexagonal shape of the wrench flats 330 of the primary screw driver 48. The socket 532 of the second handle portion 506 is configured for detachable coupling with the proximal end of the guide rod 52 or optional side screw driver 84. Accordingly, in the depicted embodiment, the socket 532 defines the hexagonal shape of the proximal end 423 of the side screw driver 84. It is recognized that each of the sockets 512, 522, and 532 may be formed to shapes other than hexagonal, to accommodate whatever shape the caps 68 and 480, wrench flats 330, and proximal end 304 may define.

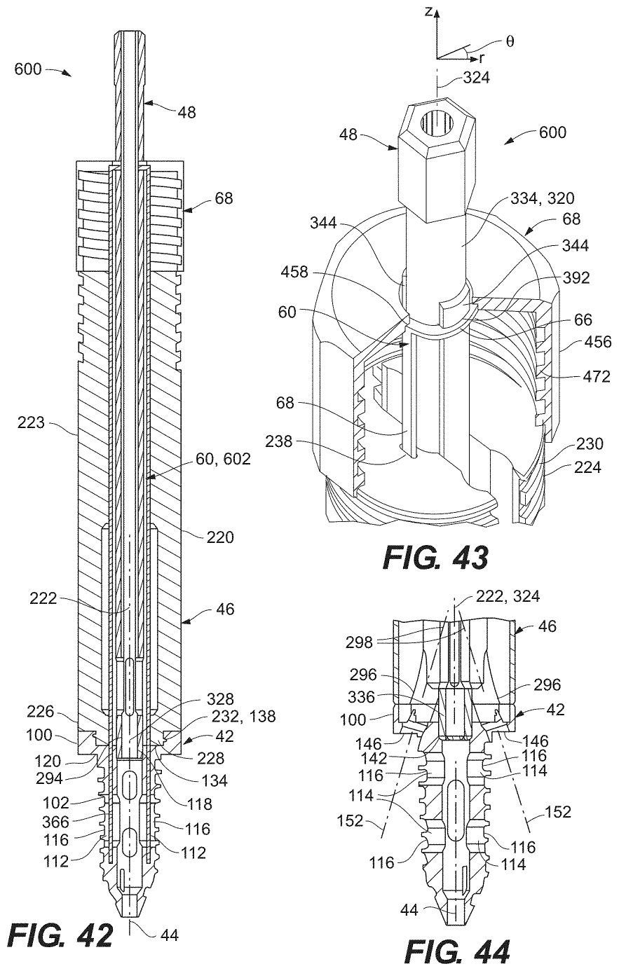

Referring to FIGS. 42 through 44, an initial assembly 600 of the primary screw 42, inserter 46, primary screw driver 48, blade assembly 60, and drive cap 68 is depicted according to an embodiment of the disclosure. For the initial assembly 600, the boss 228 of the inserter 46 is inserted into and rotated within the recess 134 of the primary screw 42 so the exterior thread 232 of the boss 228 is fully engaged with the interior thread 138 of the inner wall portions 136 of the flange 132 surrounding the recess 134. The inserter axis 222 and central egress port 294 of the main cylinder 220 is thereby aligned with the central axis 44 and opening 118 at the proximal end 120 of the body portion 102 of the primary screw 42. The threads 138, 232 may be fully engaged after, for example, a ¼ turn, ½ turn, or full turn between the inserter 46 and the primary screw 42. When the threads 138, 232 are fully engaged, the head portion 100 of the primary screw 42 registers against the distal end 226 of the main cylinder 220, effectively capping the distal end 226 and boss 228 of the inserter 46. In some embodiments, the primary screw 42 and inserter 46 are configured so that, when the threads 138, 232 are fully engaged, the canted axes 298 of the side egress ports 296 side screw ports are aligned with the side screw port axes 152 of the screw side ports 146 of the primary screw 42.

Also during the buildup of the initial assembly 600, the blade assembly 60 is disposed in the inserter 46. The elongate blades 62 are inserted into the blade passages 238 of the inserter 46 at the proximal face 242 of the main cylinder 220, through the blade passages 238 and into the blade passages 162 of the primary screw 42. For the initial assembly 600, the distal portions 366 of the elongate blades 62 extend axially through the elongate side ports 112. Also in the initial assembly 600, the blade assembly 60 defines a retracted configuration 602, wherein the distal portions 366 of the elongate blades 62 extend parallel to and are adjacent the external opening 116 of the elongate side port 112.

For the initial assembly 600, the drive cap 68 is mounted to the inserter 46. The drive cap 68 is aligned over the blade assembly 60 and the internal threads 472 of the skirt portion 456 brought into engagement with the external threads 230 at the proximal end 224 of the inserter 46. The inserter 46 and drive cap 68 are configured so that the annular recess 458 of the drive cap 68 seats on the ring 66 of the blade assembly 60 when the internal threads 472 of the skirt portion 456 are initially started on the external threads 230 of the main cylinder 220 of the inserter 46.

The primary screw driver 48 is inserted through the mounted drive cap 68. The distal end 328 of the primary screw driver 48 is inserted first, and the primary screw driver 48 rotationally oriented so that the rails 344 are aligned with the keyways 392 of the ring 66 of the blade assembly 60. The primary screw driver 48 is then further inserted until the driving head 336 reaches the primary screw 42. In some embodiments, the rails 344 are positioned on the main body 334 of the shaft portion 320 so that, when the rails 344 are aligned with the keyways 392 and the central axis 324 of the main body 334 is aligned with the central axis 44 of the primary screw 42, the driving head 336 is rotationally aligned with the shape of the socket 142 of the primary screw 42 for insertion into the socket 142. In some embodiments, upon insertion of the driving head 336 into the socket 142, the rails 344 are resident in the keyways 392 of the ring 66.

The above description refers to the blades 62, elongate side ports 112, side screw ports 146, side screw port axes 152, blade passages 162, 238, side egress ports 296, canted axes 298, rails 344, and keyways 392 in the plural. It is understood that the embodiments having a single blade 62, elongate side port 112, side screw port 146, side screw port axis 152, blade passage 162, 238, side egress port 296, canted axis 298, rail 344, or keyway 392 is also contemplated, and that accommodating such modifications, guided by the present disclosure, are readily understood by the artisan of ordinary skill.

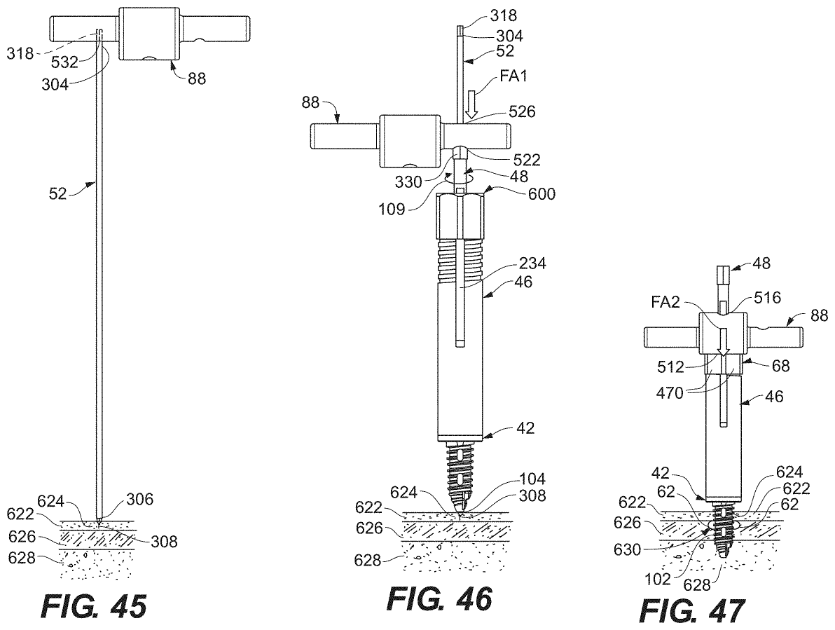

Referring to FIGS. 45 through 54, implantation of the primary screw 42 is depicted according to an embodiment of the disclosure. The distal end 306 of the guide rod 52 is placed in contact a first bone 622 (e.g., the ilium) at a desired penetration site 624. The socket 532 of the multifunctional handle 88 is coupled to the flats 318 of the proximal end 304 of the guide rod 52 and the guide rod 52 rotated with the multifunctional handle 88 to tap the self-tapping threaded structure 308 into the first bone 622 (FIG. 45).

After the guide rod 52 is anchored to the first bone 622, the multifunctional handle 88 is decoupled from flats 318 of the guide rod 52 and the initial assembly 600 slid over the guide rod 52 so that the tip portion 104 of the primary screw 42 is brought into contact with the first bone 622 at the desired penetration site 624. The socket 522 and through-aperture 526 of the multifunctional handle 88 is slid over the guide rod 52 and the socket 522 mated with the wrench flats 330 of the primary screw driver 48. An axial force FA1 is applied to the primary screw driver 48 as the primary screw driver 48 is rotated in the cutting rotational direction 109 with the multifunctional handle 88 to drive the primary screw 42 into threaded engagement with the first bone 622 (FIG. 46).

The primary screw 42 is thereby driven through the first bone 622, a tissue layer 626 (e.g., cartilage), and into a second bone 628 (e.g., the sacrum). At this point in the implantation process, the guide rod 52 may be detached from the first bone 622 and withdrawn from the inserter 46. As the elongate side ports 112 enter the tissue layer 626, the blades 62 of the blade assembly 60 may be deployed. To deploy the blades 62, the socket 512 and through-aperture 516 of the body portion 502 of the multifunctional handle 88 are slid over the primary screw driver 48 so that the socket 512 mates with the flats 470 of the drive cap 68. In some embodiments, the inserter 46 is grasped and held stationary while the drive cap 68 is drawn tight over the external threads 230 of the main cylinder 220 of the inserter 46. As the drive cap 68 is drawn onto the inserter 46, the annular recess 458 of the drive cap 68 rotates on the ring 66 of the blade assembly 60 (FIG. 43). The ring 66 is prevented from rotating by the guide 342 (e.g., rails 344) of the primary screw driver 48. Accordingly, rotation of the drive cap 68 onto the inserter 46 imparts an axial force FA2 on the blades 62 without twisting the blades 62. The axial force FA2 causes the blades 62 to deflect radially outward into a deployed configuration 630 and into the tissue layer 626 (FIG. 47). In the deployed configuration 630, the blades 62 bow radially outward through the openings 116 of the elongate side ports 112 and radially beyond the body portion 102 of the primary screw 42.

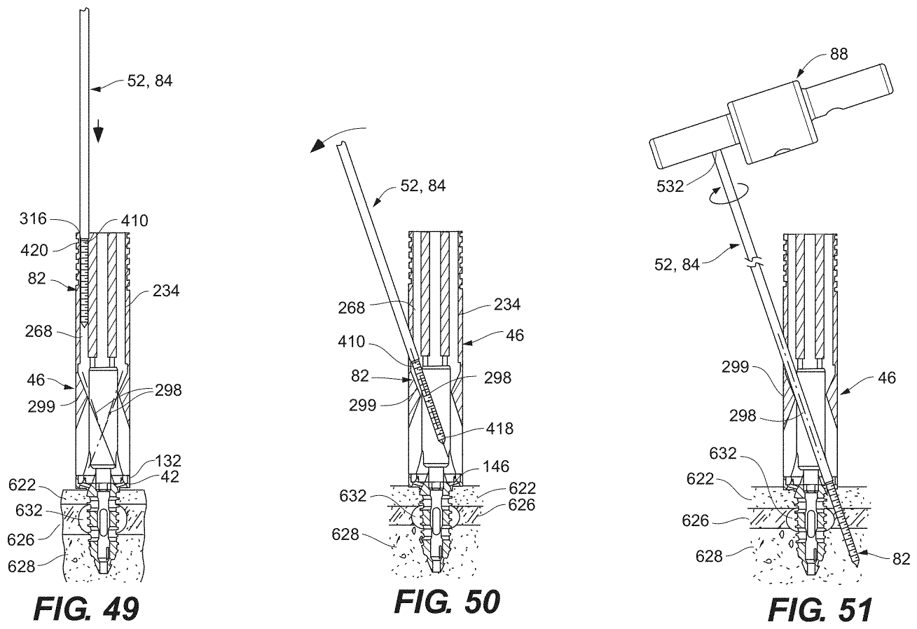

With the blades 62 in the deployed configuration 630, or while the blades 62 are being deployed, rotation of the primary screw 42 is resumed. Resumption of the rotation of the primary screw 42 may be performed by driving the inserter 46 with the multifunctional handle 88 as arranged in FIG. 47, which in turn drives the primary screw 42. Optionally, the multifunctional handle 88 may be arranged as depicted in FIG. 46 to drive the primary screw 42 with the primary screw driver 48. The rotational action of the deployed blades 62 as the primary screw 42 is driven further into the penetration site 624 cuts a zone 632 out of the tissue layer 626, which can be seen in FIG. 49. The zone 632 may be annular, surrounding the primary screw 42. In some embodiments, blades 62 remain in the deployed configuration 630 until the primary screw 42 reaches full implantation depth (i.e., until the head portion 100 of the primary screw 42 is firmly seated on the first bone 622). In other embodiments, the blades 62 are retracted before the primary screw 42 reaches full implantation depth, to prevent the blades from grinding into the second bone 628.

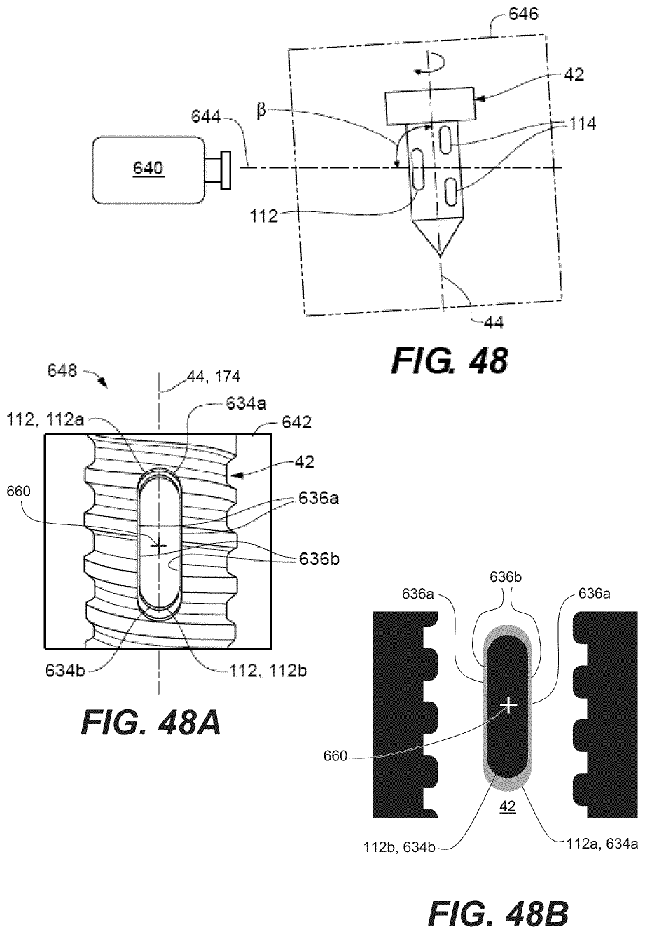

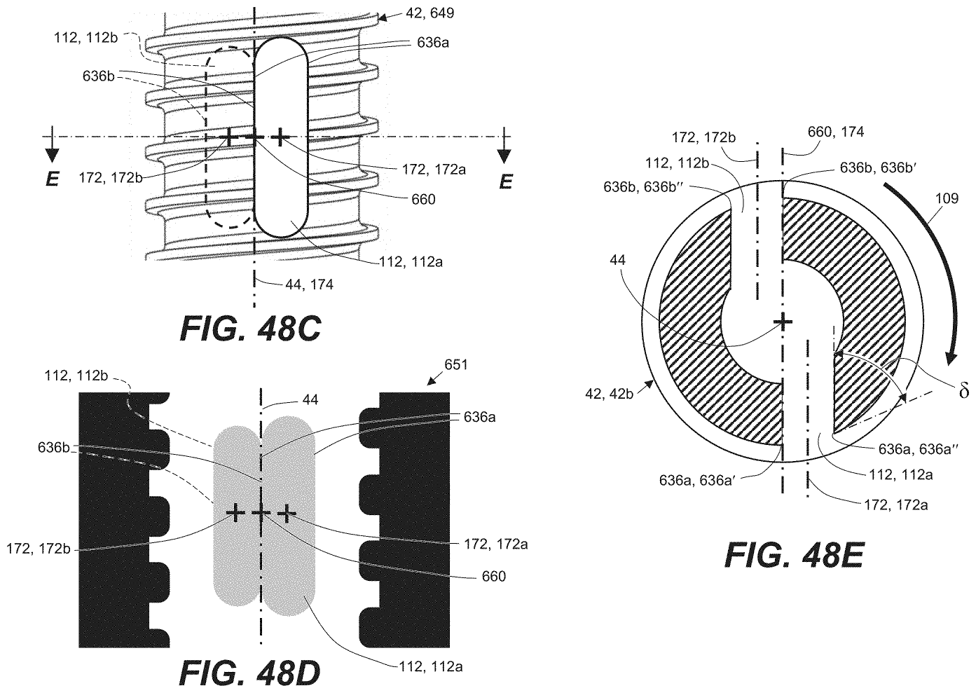

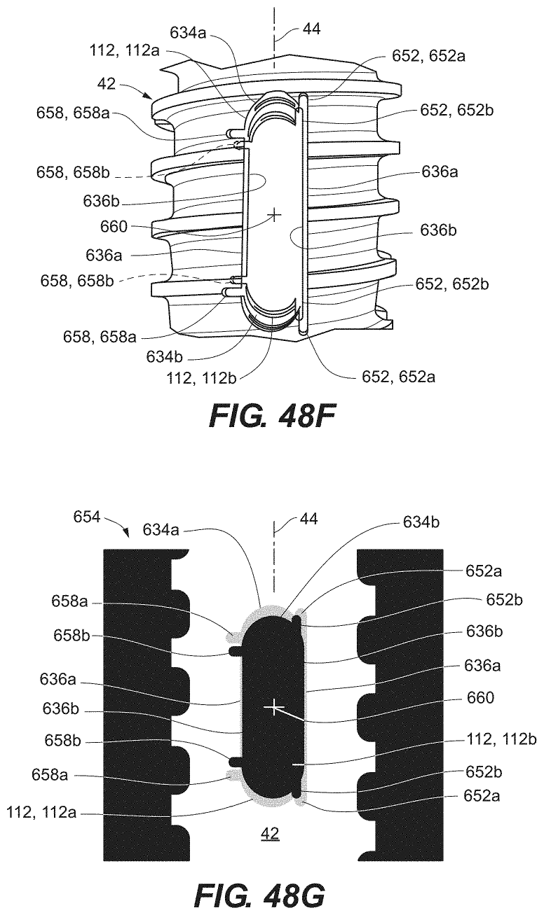

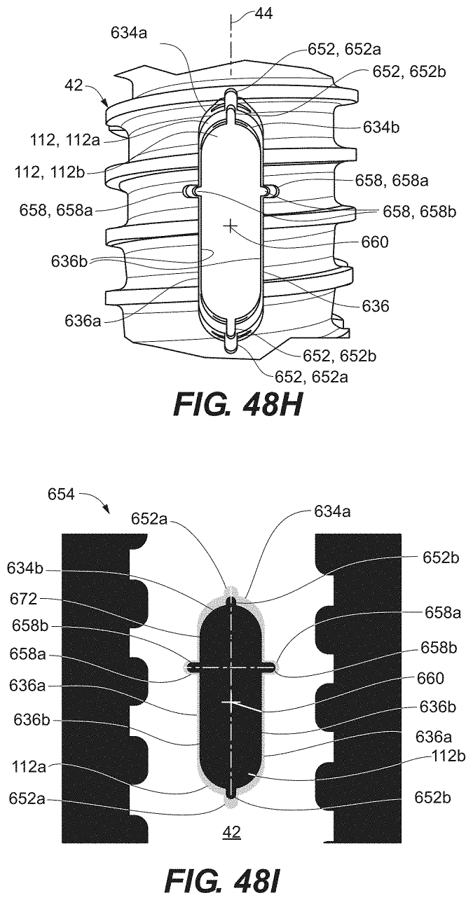

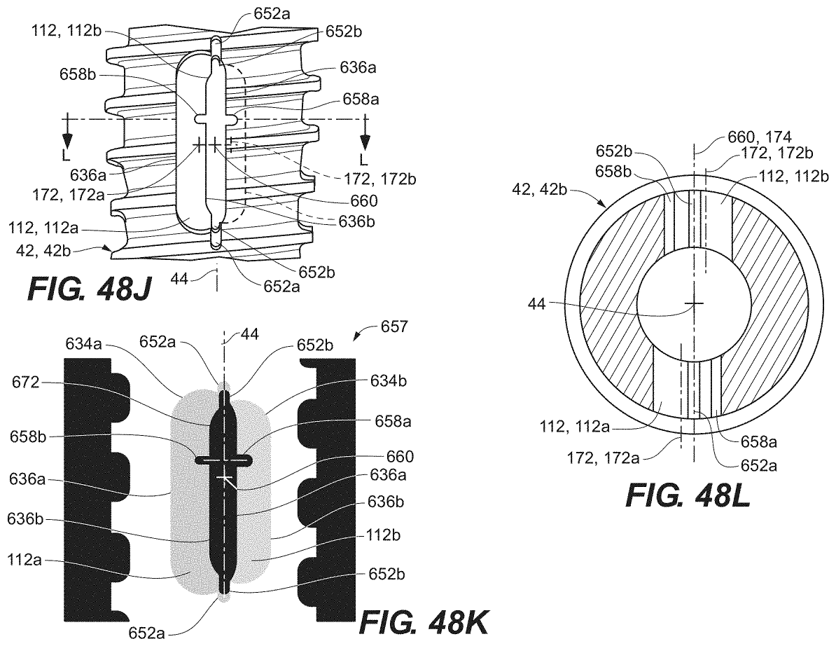

In some embodiments of the disclosure, and in reference to FIGS. 48 through 48E, a surgical imaging device 640 is utilized for rotationally aligning the primary screw 42 in a desired orientation for placement of the side screws 82 once the primary screw 42 is implanted at approximately full implantation depth. The surgical imaging device 640 defines a field of view 642 centered about a viewing axis 644.

The surgical imaging device 640 is arranged to laterally view the central axis 44 and so that the viewing axis 644 is coplanar with a desired alignment plane 646. Herein, to “laterally view” the central axis 44 is to have the central axis 44 extend across a field of view 642 of the surgical imaging device 640. In one embodiment, the desired alignment plane 646 is orthogonal to the plane of the side screw port axes 152 when the primary screw 42 is properly aligned. Alternatively, the desired alignment plane 646 may be coplanar with the plane of the side screw port axes 152 upon proper alignment. The surgical imaging device 640 is sighted along the desired alignment plane 646 so that the viewing axis 644 intersects the central axis 44 at an angle β. While the angle β preferably approximates a 90 degree angle, other angles may also be utilized. The viewing axis 644 need only be coplanar with the desired alignment plane 646 for proper alignment of the side screw port axes 152.

In some embodiments, the elongate side ports 112 are utilized for the rotational alignment of the primary screw 42. Herein, the elongate side ports 112 are identified individually as first and second elongate side ports 112a and 112b, located on the first and second lateral sides 111a and 111b (FIG. 5), respectively, of the primary screw 42. The elongate side ports 112a and 112b may be identical in shape and size, with each defining respective perimeters 634a and 634b having axially extending tangential edges 636a and 636b. In some embodiments, the tangential edges 636 are linear and extend parallel to each other (depicted).

In FIGS. 48A and 48B, the first and second elongate side ports 112a and 112b are depicted as being centered in diametric opposition along a central lateral axis 660 that passes through the central axis 44 and is coplanar with the mid-plane 174. The second elongate side port 112b, which is furthest from the surgical imaging device 640, appears to be within the first elongate side port 112a, which is nearer the surgical imaging device 640, even though the side ports 112a and 112b may be of identical dimension. The appearance of the side port 112b being within the side port 112a in two dimensions arises because of the focal depth of the surgical imaging device 640, Certain candidate materials for the primary screw 42, such as titanium, are known to be semi-transparent to x-rays. More of the x-rays that pass through thicker or multiple thicknesses of material will be absorbed or otherwise attenuated by the material, while x-rays that pass through no material experience only light or incidental attenuation.

To rotationally align the primary screw 42 in the desired orientation using the elongate side ports 112, the primary screw 42 is rotated so that the first lateral side port 112a nearest the surgical imaging device subtends the viewing axis 644. The primary screw 42 is then rotationally adjusted until both of the tangential edges 636b of the second lateral side port 112b are visible through the first lateral side port 112a. An example of an aligned orientation 648 of the lateral side ports 112 is presented in FIG. 48A for a primary screw 42 that is rotationally aligned along the viewing axis 644. A representation of a corresponding image 650 produced by the surgical imaging device 640 is depicted in FIG. 48B for an x-ray imaging device.

In FIGS. 48C through 48E, the first and second elongate side ports 112a and 112b are depicted as centered about their respective offset lateral axes 172a and 172b that extend parallel to the central lateral axis 660 and mid-plane 174 and but are laterally offset from the central lateral axis 660 and central axis 44, akin to the primary screw 42b of FIG. 8. Unlike the primary screw 42b of FIG. 8, a first axially extending tangential edge 636a′ of the axially extending tangential edges 636a and a first axially extending tangential edge 636b′ of the axially extending tangential edges 636b are coplanar with each other, and may be coplanar with the central axis 44 and the mid-plane 174 (depicted).

An example of an aligned orientation 649 is presented in FIG. 48C for the central lateral axis 660 of primary screw 42 rotationally substantially aligned along the viewing axis 644. A representation of a corresponding image 651 produced by the surgical imaging device 640 is depicted in FIG. 48D for an x-ray imaging device. The appearance of the side port 112b being smaller than the side port 112a in two dimensions arises because of the focal depth of the surgical imaging device 640. In some embodiments, proper rotational alignment of the primary screw 42 causes the x-rays that pass through each of the side ports 112 to pass through a single wall of the primary screw 42 semi-transparent to x-rays, such that the x-rays absorbed or otherwise attenuated by the material are substantially the same.

The positioning of the ports 112 so that the first axially extending tangential edges 636a′ and 636b′ are the leading edges of the ports 112 when the primary screw 42 is rotated in the cutting rotational direction 109. When configured in this way, second axially extending tangential edges 636a″ and 636b″ form the cutting edges of the ports 112 that define a cutting angle δ (FIG. 84E). Because the cutting edges follow the mid-plane 174 of the primary screw 42 when rotated in the cutting rotational direction 109, the cutting angle δ is more aggressive than if the first axially extending tangential edges 636a′ and 636b′ were the trailing edges of the ports 112. This aspect is described in greater detail attendant to FIGS. 67 through 69 below.

To rotationally align the primary screw 42 in the desired orientation using the offset elongate side ports 112 of FIG. 48C, the primary screw 42 is rotated so that the first axially extending tangential edges 636a′ and 636b′ as seen in the image 651 are substantially aligned as viewed along the viewing axis 644. If the first axially extending tangential edges 636a′ and 636b′ overlap, the image 651 may indicate no attenuation of the x-rays (i.e., a darker appearance at the confluence than depicted in FIG. 48D); if the first axially extending tangential edges 636a′ and 636b′ do not overlap but are still separated, the image 651 may indicate double attenuation of the x-rays (i.e., a lighter appearance at the gap between edges 636a′ and 636b′ than depicted in FIG. 48D).

In some embodiments, and in reference to FIGS. 48F through 48L, each of the perimeters 634a and 634b define a pair of axial notches 652a and 652b, respectively (referred to collectively or generically as axial notches 652). The axial notches 652 extend substantially parallel to the central axis 44 of the primary screw 42. The axial notches 652a and 652b are depicted in FIG. 48F as extending axially from the tangential edges 636a and 636b, respectively, with a representation of a respective corresponding image 654 depicted in FIG. 48G.

In FIG. 48H, both lateral side ports 112a and 112b and both pairs of axial notches 652a and 652b are depicted as being centered in diametric opposition along the central lateral axis 660 that passes through the central axis 44 and is coplanar with the mid-plane 174. As such, the axial notch pairs 652a and 652b are tangentially centered with respect to the perimeters 634a and 634b, respectively. A representation of a respective image 656 corresponding to the view of FIG. 48H is depicted in FIG. 48I.

In FIG. 48J, which portrays alignment of primary screw 42b of FIGS. 8 through 8B, the lateral side ports 112a and 112b are centered about their respective offset lateral axes 172a and 172b that are laterally offset from the central axis 44. However, the axial notch pairs 652a and 652b are centered in diametric opposition along the central lateral axis 660 that passes through the central axis 44. The relationship between the offset lateral axes 172a and 172b and the central lateral axis 660 is depicted in FIG. 48L. As such, the axial notch pairs 652a and 652b are not centered with respect to the respective lateral side port 112a and 112b. A representation of a respective image 657 corresponding to the view of FIG. 48J is depicted in FIG. 48K.

In some embodiments, each of the perimeters 634a and 634b define at least one tangential notch 658a and 658b, respectively (referred to collectively or generically as tangential notch(es) 658). The tangential notch(es) 658 extend substantially orthogonal to the central axis 44 of the primary screw 42. In FIG. 48F, the tangential notches 658a and 658b are depicted as extending tangentially from one of the tangential edges 636a and 636b, respectively, with a representation of the respective corresponding image 654 depicted in FIG. 48G.

In FIG. 48H, the tangential notches 658a and 658b are depicted as being axially centered with respect to the perimeters 634a and 634b, respectively, and extending from both the tangential edges 636a and 636b, respectively, with the representation of the respective corresponding image 656 depicted in FIG. 48I. The axial notches 652 and the tangential notches 658 of FIGS. 48H and 48I in effect represent the axial and lateral ends of a reticle pattern 672, depicted with dashed lines in FIG. 48I.

In FIGS. 48J and 48L, a single tangential notch 658a is defined as extending from one of the tangential edges 636a. The tangential edge 636a from which the tangential notch 658a extends is the tangential edge 636a that is closer to the central lateral axis 660. Likewise, a single tangential notch 658b is defined as extending from one of the tangential edges 636b, with the tangential edge 636b from which the tangential notch 658a extends being the tangential edge 636a that is closer to the central lateral axis 660. The representation of the respective corresponding image 657 depicted in FIG. 48K. When aligned along the central lateral axis 660, the axial notches 652 and the tangential notches 658 of FIGS. 48J and 48K in effect represent the axial and lateral ends of the reticle pattern 672, depicted with dashed lines in FIG. 48L.

Functionally, the axial notches 652, when implemented, assist in the rotational alignment of the primary screw 42. The primary screw 42 is rotated so that the lateral side port 112a nearest the surgical imaging device subtends the viewing axis 644. The primary screw 42 is then tweaked rotationally so that the axial notches 652a and the axial notches 652b are in axial alignment. Examples of aligned orientations of the axial notches 652 is presented in FIGS. 48F, 48H, and 48J for a primary screw 42 that is rotationally aligned along the viewing axis 644. Representations of corresponding images 654, 656, and 657 produced by the surgical imaging device 640 are depicted in FIGS. 48G, 48I, and 48K, respectively for an x-ray imaging device.

Note that for the primary screw 42b of FIG. 48J, alignment of the perimeters 634a and 634b of the lateral side ports 112a and 112b along the viewing axis 644 does not rotationally align the side screw ports 146 of the primary screw 42b. This is because the offset lateral axes 172a and 172b of the lateral side ports 112a and 112b are laterally offset relative to the central axis 44 and the central lateral axis 660. Accordingly, if the lateral side ports 112a and 112b were aligned along the viewing axis 644, the viewing axis 644 would not be aligned with the desired alignment plane 646. The axial notches 652, however, provide a feature such that, when the primary screw 42b is rotated so that the axial notches 652 are coplanar with the viewing axis 644, the viewing axis 644 is also coplanar with the central lateral 660 and the desired alignment plane 646.

Once the primary screw 42 is rotationally aligned, the separation between corresponding tangential notches 658 (when implemented) provides an indication of the pitch of the primary screw 42 with respect to the viewing axis 644. The image 654 of FIG. 48G depicts the tangential notches 658a and 658b as having small axial separation 674, indicating that the angle β between the central axis 44 of the primary screw 42 and the viewing axis 644 is approximately 90 degrees. The further the angle β deviates from 90 degrees, the greater the separation between the tangential notches 658.

The image 656 of FIG. 48I depicts the tangential notches 658a and 658b as being in alignment (i.e., as having essentially no axial separation) for the reticle pattern 672, indicating that the angle β between the central axis 44 of the primary screw 42 and the viewing axis 644 is approximately 90 degrees. The further the angle β deviates from 90 degrees, the greater the separation between the tangential notches 658 of the reticle pattern 672.

The representative images 650, 651, 654, 656, and 657 of FIGS. 48B, 48D, 48G, 48I, and 48K, respectively, illustrate the effect of the semi-transparency of the material of the primary screw 42 to x-rays. The regions where x-rays pass through more than one wall thickness of screw material (e.g., that pass twice through the side wall 106 of the primary screw 42) are represented in white. Regions where x-rays do not pass through any of the screw material are represented in black. Regions where x-rays pass through only one wall thickness of screw material are represented in gray. While representative images 650, 651, 654, 656, and 657 of FIGS. 48B, 48D, 48G, 48I, and 48K are not exact or photographic depictions of a screw in an x-ray image, those of skill in the relevant arts will understand what these depictions represent—that the various locations of the perimeters 634b are discernable from the perimeters 634a in the ways described with the surgical imaging device 640. The ability to distinguish perimeters 634a and 634b using the methods described provides enhanced rotational alignment capability of the various primary screws 42.

In some embodiments, the foregoing methods are outlined on the instructions 92. The instructions 92 may be physically included with the kit 90 such as on a printed document (depicted), compact disc, or flash drive. In other embodiments, the instructions 92 may be provided remotely, for example on a hard drive of a remote server that is internet accessible with an electronic device such as a computer, smart phone, or electronic tablet. The instructions 92 may include text, photos, videos, or a combination thereof to instruct and guide the user.

Once the primary screw 42 is at full implantation depth and rotationally oriented as desired, the drive cap 68, blade assembly 60, and primary screw driver 48 may be removed from the inserter 46 for installation of the side screws 82. The drive cap 68 is removed from the proximal end of the inserter 46, so that the elasticity of the blades 62 cause the blades 62 to return to a straight configuration. The primary screw driver 48 and blade assembly 60 are extracted proximally from the inserter 46, leaving only the inserter 46 and the primary screw 42. In some embodiments, the guide rod 52 is utilized to route and set the side screws 82. The driving head 316 of the guide rod 52 is press fit into the socket 420 of a first of the side screws 82. Alternatively, the side screw driver 84 is used instead, with the driving head 428 of the side screw driver 84 forming the press fit with the socket 420 the side screw 82. Using the guide rod 52 or side screw driver 84, the side screw 82 is inserted into the access slot 234 so that the head 410 of the side screw 82 is captured and guided by a first of the mirrored side arcuate channels 268 of the opposed inner surfaces 264 of the access slot 234 (FIG. 49). The side screw 82 is translated axially through the mirrored side arcuate channels 268 until encountering a first of the guide ramps 299. At the guide ramp 299, the distal end 418 of the side screw 82 is rotated laterally inward with the guide rod 52 or side screw driver 84 as the head 410 is slid axially through the mirrored side arcuate channels 268 until the side screw 82 is substantially aligned with a first of the canted axes 298 (FIG. 50). The side screw 82 is then translated along the canted axis 298 to a first of the side screw ports 146. The socket 532 of the multifunctional handle 88 is fitted to the guide rod 52 or side screw driver 84 to drive and set the side screw 82 through the first bone 622, tissue 626, and second bone 628 (FIG. 44). Because the side screw 82 is firmly implanted in the bones 622 and 628, the light press fit between the driving head 316 of the guide rod 52 and the socket 420 of the side screws 82 is readily overcome by pulling the guide rod 52 out of the head 410 of the side screw 82. For the depicted embodiment, the procedure is repeated for the implantation of a second of the side screws 82, using a second of the mirrored side arcuate channels 268, a second of the guide ramps 299, a second of the canted axes 298, and a second of the side screw ports 146.

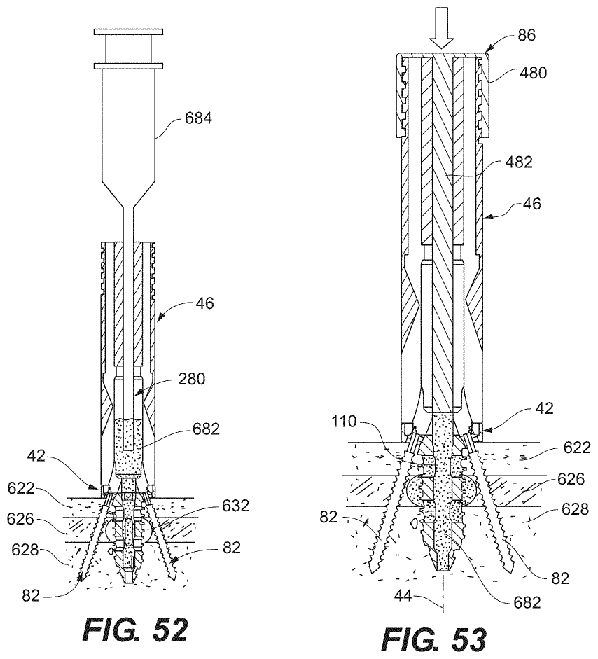

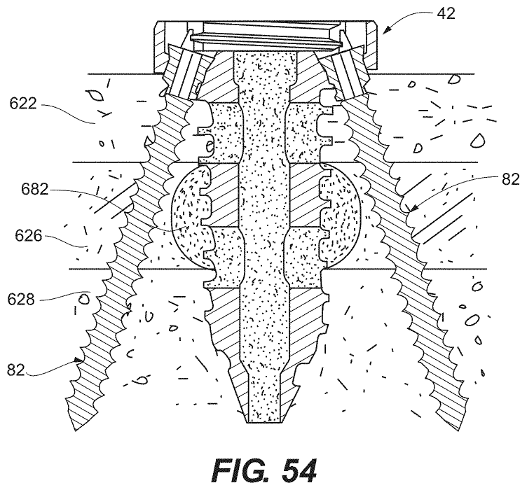

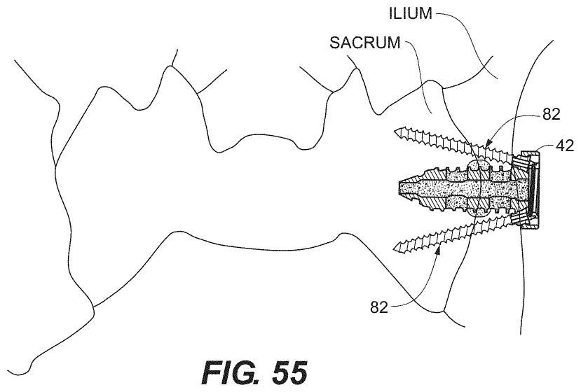

A portion of the tissue dislodged by the self-tapping primary screw 42 and the cutting action of the blades 62 may be internalized or drawn into the primary screw 42, for example by the rotating action of the blades 62 and the elongate side ports 112, as well as by retraction of the blades 62 back into the elongate side ports 112. Bone grafting material 682 may be injected into the primary screw 42 and the interior chamber 280, for example with a syringe 684 (FIG. 52). In some embodiments, the bone grafting material 682 is distributed and packed into the zone 632 via the side ports 112, 114 using the plunger assembly 86. The plunger stem 482 is inserted through the mirrored central arcuate channels 266 and into the interior chamber 280 of the inserter 46. The cap 480 is then threaded onto the external threads 230 of the main cylinder 220 to drive the plunger stem 482 into the bone grafting material 682, thereby pressurizing and packing the bone grafting material 682 into the zone 632 as well as the interior chamber 110 of the primary screw 42 (FIG. 53). The inserter 46 and plunger assembly 86 may be removed from the primary screw 42 by rotating the inserter 46 counterclockwise about the central axis 44 to decouple the exterior thread 232 of the boss 228 from the interior thread 138 of the recess 134 of the primary screw 42. The final, implanted assembly is left behind, as depicted in FIGS. 54 and 55.

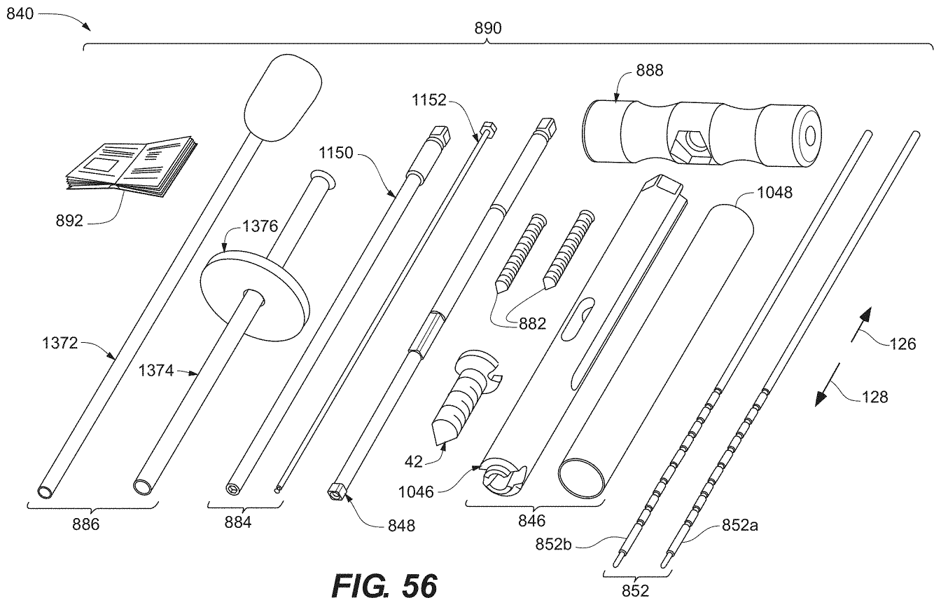

Referring to FIG. 56, an implant system 840 for fusion of a sacroiliac joint is depicted according to an embodiment of the disclosure. The implant system 840 includes the main or primary screw 42 configured to detachably mate with an inserter assembly 846. In some embodiments, a main or primary screw driver 848 is configured to access the primary screw 42 through the inserter assembly 846. The primary screw 42, the inserter assembly 846, and the primary screw driver 848 may be configured for sliding over a guide wire or rod 852. The implant system 840 may include one or more side screws 882, a side screw driver assembly 884, a plunger assembly 886, and a multifunctional handle 888. In some embodiments, the some or all of the components of the implant system 840 are provided as a kit 890, including operating instructions 892 that are provided on a tangible, non-transitory medium. Additional details, functional descriptions, and methods of use for the various components of the implant system 840 are described below.

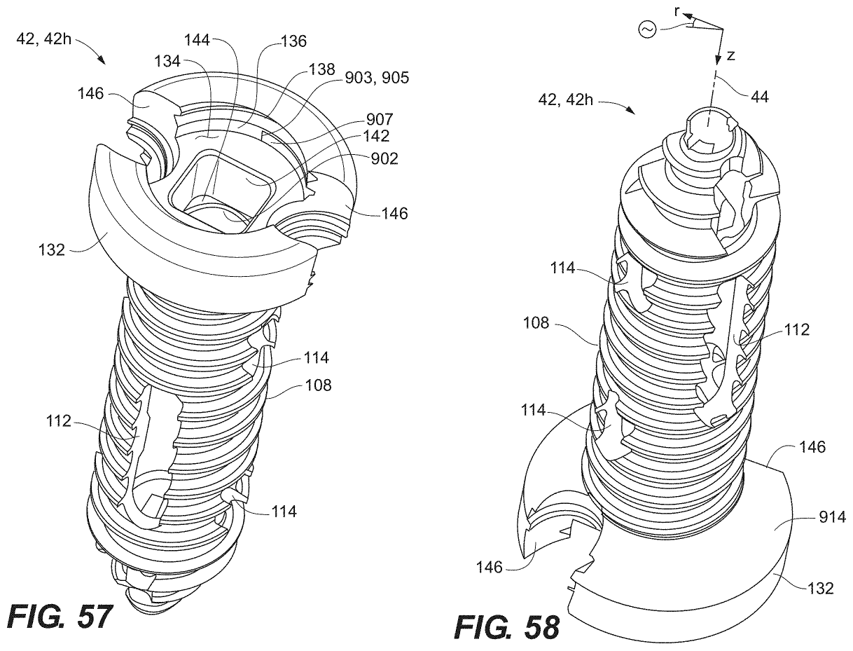

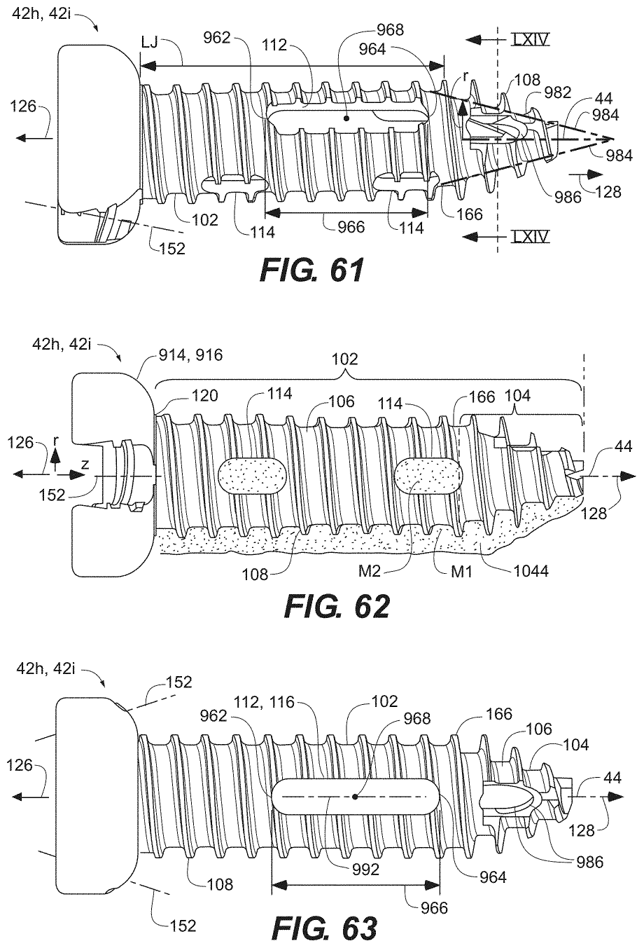

Referring to FIGS. 57 through 68, primary screws 42h and 42i are depicted according to embodiments of the disclosure. The primary screws 42h, 42i variously include some of the same components and attributes as other the primary screws 42 disclosed herein, some of which are indicated by same-labeled reference characters. Furthermore, any of the specific configurations of a given component or attribute disclosed for other primary screws 42 may be incorporated into the primary screws 42h, 42i. For example, the side screw ports 146 may be configured to include the attributes of side screw ports 146.1 (FIGS. 32-33A), 146.2 (FIGS. 34A, 34B), 146g (FIGS. 37A, 37B), or any of the other side screw ports disclosed herein. Other non-limiting examples include: the external threads 108 may be configured in accordance with 108a (FIGS. 2-7), 108c (FIG. 9), 108d (FIG. 10A), 108e (FIG. 10B), or any of the other external threads disclosed herein; and the side ports 112 may be configured in accordance with any one of FIGS. 8A, 8B, and 48A through 48L.

For the depicted embodiment of the primary screw 42h, 42i, the socket 142 is of a rounded-corner square shape (i.e., defining a substantially square recess having rounded corners). An aperture 902 is defined at a proximal end 904 of the interior chamber 110 having a diameter 906 that is sized for passage of the guide rod 52. In some embodiments, the diameter 906 of the aperture 902 is less than a maximum radial dimension of the socket 142, thereby defining the registration surface 144 at the interface of the socket 142 and the interior chamber 110. In some embodiments, the interior chamber 110 defines a diameter 908 distal to the aperture 902 that is larger than the diameter 906.

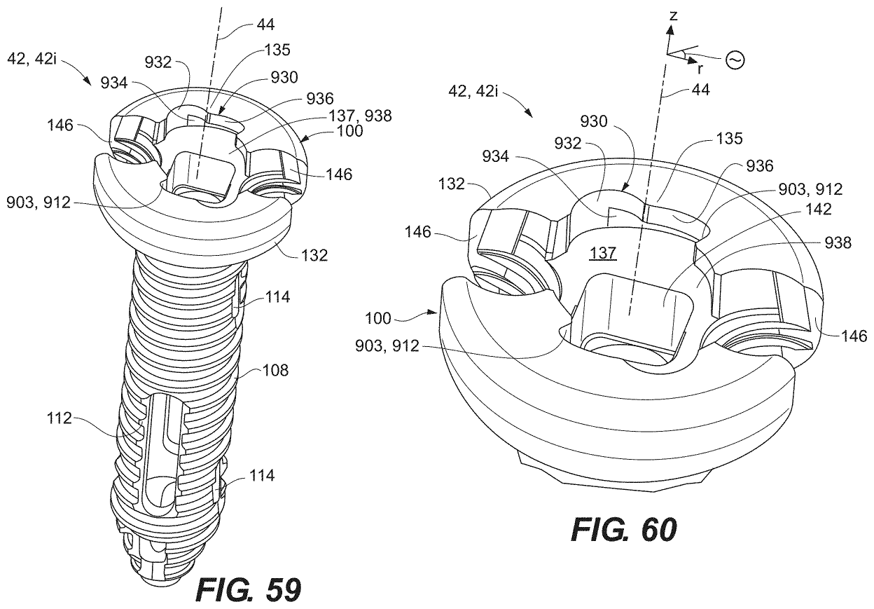

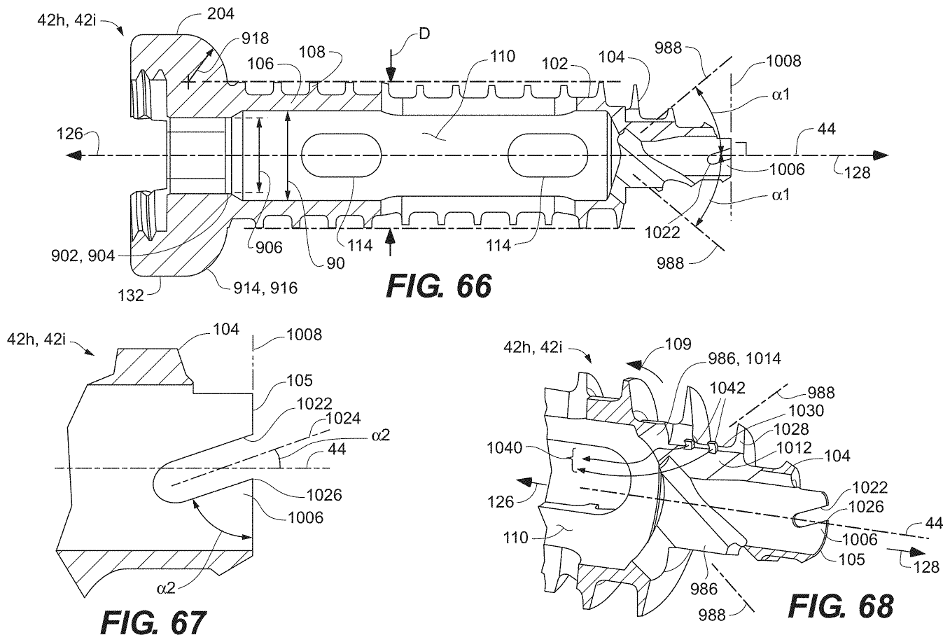

In some embodiments, the inner wall portion 136 of the flange 132 includes a tangential stop 903 against which the inserter assembly 846 is rotationally registered within the recess 134. For the primary screw 42h, the tangential stop 903 takes the form of an abrupt termination 905 of a distal end 907 the interior thread 138 formed on the inner wall portion 136 of the flange 132 (FIG. 57). For the primary screw 42i, the tangential stop 903 is a radial flat 912 that extends radially from the inner wall portion 136 (FIGS. 59 and 60). Herein, a “radial flat” is coplanar with a plane that extends in an axial direction and a radial direction (e.g., defines a plane in the r and z coordinates of the r-θ-z coordinate system of the primary screw 42). In some embodiments, a distal face 914 of the flange 132 of the primary screw 42h, 42i has a radiused profile 916 that defines a radius 918 (FIG. 66). In some embodiments, the radius 918 within a range of 2 millimeters to 5 millimeters inclusive. In some embodiments, the radius 918 extends from the major diameter D of the threads 108 to the radial outward face 204 of the flange 132.

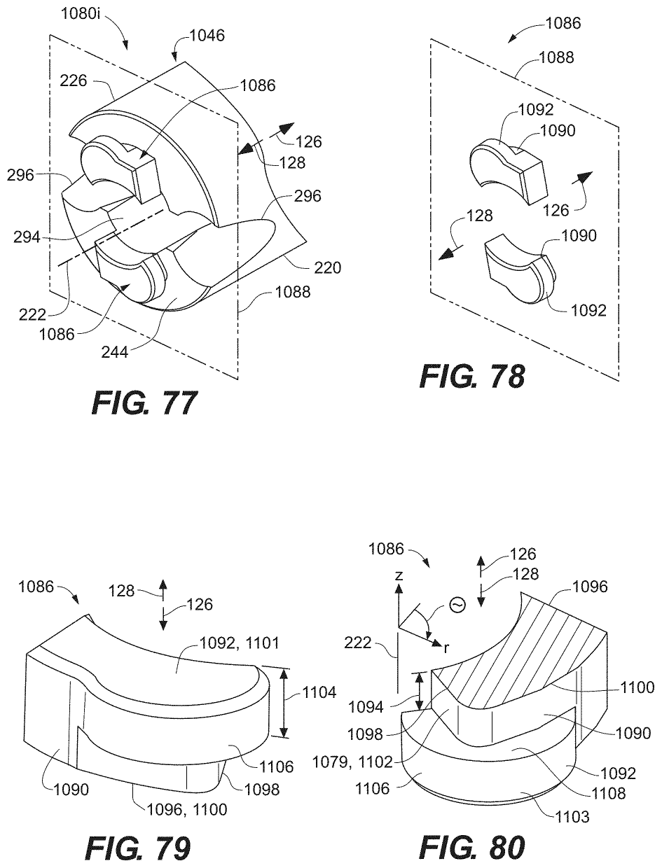

The primary screw 42i is described in greater detail in reference to FIGS. 59 and 60 according to an embodiment of the disclosure. The flange 132 of the head portion 100 includes an interlocking structure 930 for coupling the primary screw 42i with embodiments of the inserter 1046 (FIG. 77). The interlocking structure 930 defines an access 932 that extends from the exterior proximal face 135 to the interior proximal face 137 of the head portion 100. The interlocking structure 930 defines an undercut 934 adjacent the access 932 and includes a jut 936 that extends axially from the exterior proximal face 135 and radially inward over the undercut 934. The access 932 and undercut 934 may terminate distally at the interior proximal face 137 to define a plane 938. In some embodiments, the jut 936 includes the radial flat 912 that extends radially to define the tangential stop 903. For the primary screw 42i, the interlocking structure 930 is defined at the flange 132 between the side screw ports 146. In some embodiments, the head portion 100 includes two interlocking structures 930, which may be diametrically opposed to each other about the central axis 44.

The elongate side port(s) 112 includes a proximal end 962 and a distal end 964, defining an axial length 966 that is centered about an axial center point 968. The axial center point 968 may be located such that the distal end 964 of the elongate side port(s) 112 is closer to the tip junction 166 of the body portion 102 of the primary screw 42h, 42i than is the proximal end 962 of the elongate side port(s) 112 to the proximal end 120 of the body portion 102. In some embodiments, the axial length 966 of the elongate side port(s) 112 is within a range of 20 millimeters to 60 millimeters inclusive. In some embodiments, a ratio of the axial length 966 of the elongate side port(s) 112 to a length LJ from the proximal end 120 to the tip junction 166 of the body portion 102 is within a range of 20% to 60% inclusive. In some embodiments, the center point 968 is located within the distal ⅓ to the distal ½ of the length LJ. In some embodiments, the center point 968 is located within the distal 15% to the distal 45% of the length LJ.

The side ports 112 and 114 of the primary screws 42h, 42i may incorporate one or more of the various configurations and aspects limned for other primary screws 42 disclosed herein. For example, the side ports 112, 114 may extend along axes that are laterally offset relative to the central axis 44, akin to offset axes 174 at FIG. 8A. Alternatively or in addition, the trailing edge of the side ports 112, 114 may define the acute sweeping angle y, such as depicted and described attendant to FIG. 8B. The side ports 112 may also include alignment aspects, such as the rotational alignment notches 652, 658, of FIGS. 48A through 48L.

In some embodiments, the external threads 108 of the tip portion 104 of the primary screw 42h, 42i includes a root portion 982 that defines radii r perpendicular to the central axis 44 that decrease along the central axis 44 in the distal direction 128 so that the root portion 982 of the external threads 108 at the tip portion 104 defines a tapered profile 984 that declines toward the central axis 44 in the distal direction 128. The tip portion 104 may define at least one distal side port 986 that extends through the side wall 106 along a distal side port axis 988, the distal side port 986 being in fluid communication with the interior chamber 110.

The body portion 102 defines a laterally extending mid-plane 992 that is coplanar with the central axis 44. In some embodiments, each distal side port 986 is centered about a respective distal side port axis 988 that is parallel to but not coplanar with the laterally extending mid-plane 992, thereby defining a lateral offset 996 that extends orthogonally from the laterally extending mid-plane 992 to the distal side port axis 988. In some embodiments, the distal side port axis 988 projects onto the central axis 44 in a direction orthogonal to the laterally extending mid-plane 992 to define an acute angle al between the distal side port axis 988 and the central axis 44 (FIG. 66). Here, to “project” the distal side port axis 988 onto the central axis 44 is to view the distal side port axis 988 and the central axis 44 isometrically in a direction orthogonal to the laterally extending mid-plane 992. Accordingly, even though the distal side port axis 988 and the central axis 44 may not intersect, the angle al is “projected” as between the distal side port axis 988 and the central axis 44. The acute angle al is open to the distal direction 128.

In some embodiments, two distal side ports 986a and 986b are defined about distal side port axes 988a and 988b, respectively. The lateral offset 996 of the distal side port axes 988a and 988b may be of equal distance from the laterally extending mid-plane 992. In some embodiments, the distal side port axes 988a and 988b are offset from the laterally extending mid-plane 992 in opposite directions.

In some embodiments, the tip portion defines at least one cutting tooth 1006 at the distal extremity 105 of the tip portion 104. The distal extremity may define a distal extremity plane 1008 that is orthogonal to the central axis 44. In some embodiments, the confluence of the tapered profile 984 and the opening 107 defines a sharp edge at the distal extremity 105. The cutting tooth 1006 may be formed by a relief slot 1022 defined by the tip portion 104, the relief slot 1022 being open to the distal extremity 105 of the tip portion 104 and defining an elongate axis 1024 that extends in the distal direction 128. A cutting point 1026 is defined at the confluence of the distal extremity 105 and the relief slot 1022. The relief slot 1022 may define a right angle at the cutting point 1026 (e.g., FIG. 35A), an obtuse angle (not depicted), or an acute angle α2 (FIG. 67).

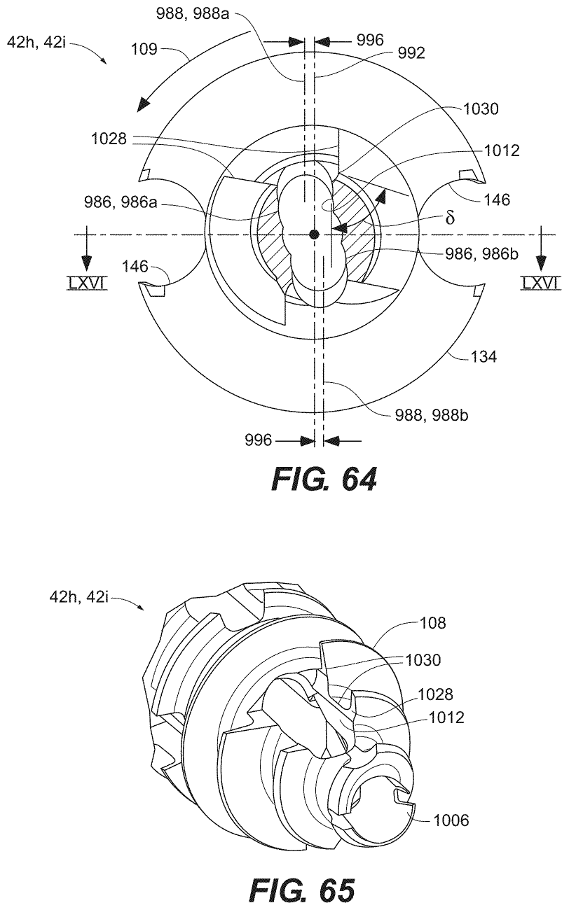

In some embodiments, the tip portion 104 defines a flute 1028 (FIG. 64). The flute 1028 forms a cutting edge 1030 that outlines at least some of the threads 108 of the tip portion 104 (FIG. 65). The distal side port 986 may extend through the flute 1028. A sweeping face 1012 is thereby defined on a portion of the distal side port 986.

Functionally, the tip portion 104 effectively acts as a pre-drill ahead of the remainder of the body 102 of the primary screw 42, but instead of the displaced bone tissue being removed from the penetration site 624, a portion of the displaced bone tissue is internalized within the primary screw 42 as autograft. Forming the distal side port 986 to extend through the flute 1028 augments this process, acting to channel some of the tissue that is cut by the flute 1028 into the distal side port(s) 986. Example sweep paths 1040 of illustrative tissue fragments 1042 are depicted in FIG. 68. Rotating the primary screw 42h, 42i in the cutting rotational direction 109 causes the cutting edge 1030 to shave off the bone tissue as the primary screw 42h, 42i is rotated and traversed axially in the distal direction 128, thereby forming the tissue fragments 1042. The sweeping face 1012 gathers and directs the tissue fragments 1042 radially inward toward the interior chamber 110 as the primary screw 42h, 42i continues through rotation and axial traversal. The acute angle al of the distal side port axis 988 defines an axially trailing face 1014 of the distal side port 986 that declines toward the central axis 44 in the proximal direction, thereby aiding in the throughput of the tissue fragments 1042 to prevent fouling of the distal side port 986.

As the tip portion 104 traverses through the bone, not all of the bone tissue that interacts with the cutting edge 1030 is shaved and diverted into the distal side port 986. A compression zone 1044 of the bone tissue surrounds and is adjacent the primary screw 42h, 42i remains connected to the greater bone as the threads 108 cut through the bone tissue (FIG. 62). As the primary screw 42h, 42i rotates and axially traverses in the distal direction 128, the tapered profile 984 of the tip portion 104 pushes and further compresses the bone tissue within the compression zone 1044 radially outward. Accordingly, the bone tissue adjacent the primary screw 42h, 42i becomes progressively compressed to increase the density of the compression zone 1044 as the tip portion 104 passes through the bone tissue. The compression zone 1044 may increase the density of the bone tissue surrounding the threads 108 of the body portion 102 for enhanced anchoring of the primary screw 42h, 42i.

Accordingly, the effect of implementing the implant system 840 is to capture a first mass M1 of the bone tissue displaced by the primary screw 42h, 42i while diverting a second mass M2 of the bone tissue displaced by the primary screw 42h, 42i into the compression zone 1044 surrounding the primary screw 42h, 42i. Herein, the “bone tissue displaced by the primary screw” refers to the total mass M of bone tissue that originally occupied the volume of the body portion 102 of the primary screw, including the side wall 106 and interior chamber 110, where M=M1+M2.

The cutting tooth 1006 at the distal extremity 105 of the tip portion 104 functions to initially pilot the self-tapping of the primary screw 42h, 42i. An axial force is applied to the primary screw 42h, 42i in the distal direction 128 as the primary screw 42h, 42i is rotated, causing the cutting point 1026 to burrow into the cortical layer of the bone.

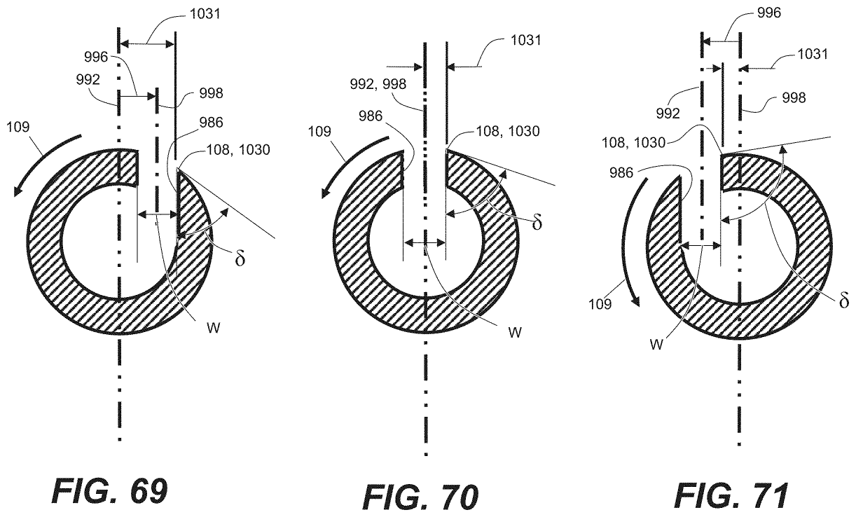

Referring to FIGS. 69 through 71, the effect of the offset 996 is schematically depicted according to embodiments of the disclosure. The cutting angle δ is depicted at the root of the threads 108 along the cutting edge 1030. (The cutting angle δ is also overlaid onto the sectional views of FIGS. 48E and 62.) In some embodiments, the distal side port 986 defines the cutting angle δ at the root of the threads 108. A lateral distance 1031 is defined between the mid-plane 992 and the cutting edge 1030. For a distal side port 986 of a given lateral or width dimension W, the magnitude and direction of the offset 996 affects the size of the cutting angle δ by affecting the lateral distance 1031. Consider a configuration where the distal side port 986 is offset so that the lateral distance 1031 to the cutting edge 1030 trails the mid-plane 992 in the cutting rotational direction 109 (FIGS. 69 and 70). The cutting angle δ thereby defined will be less than 90 degrees.

The greater the lateral distance 1031 in the trailing direction, the smaller the cutting angle S. For configurations where the distal side port 986 is offset so that the cutting edge 1030 leads the mid-plane 992 in the cutting rotational direction 109 (FIG. 71), the cutting angle δ thereby defined will be greater than 90 degrees. Accordingly, in general, the further the cutting edge 1030 is positioned tangentially in the cutting rotational direction 109, the greater the cutting angle δ at the root of the threads 108. As such, one can tailor the cutting angle δ of the cutting edge 1030 in the manner described by simply locating the position of the distal side port 986, without need for special manufacturing techniques to effect the cutting angle δ.

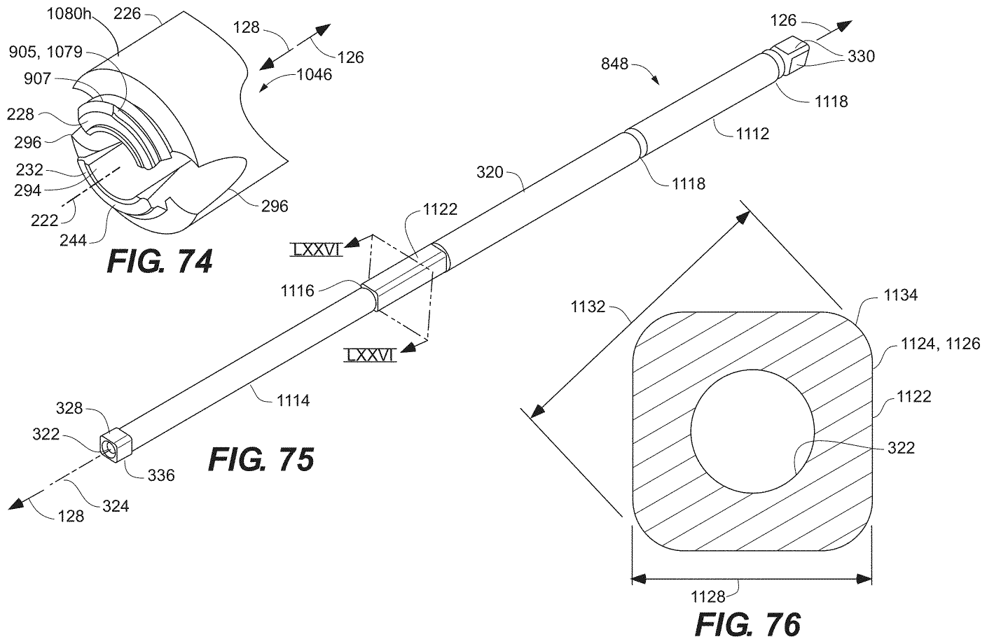

The socket 142 and correspondingly smaller diameter 906 of the aperture 902 is sized to provide sufficient material thickness between the socket 142 and the screw port(s) 146. The larger diameter 908 of the interior chamber 110 distal to the aperture 902 enables the primary screw 42h, 42i to accommodate more live bone tissue fragments or biologic for enhanced ingrowth of the primary screw 42h, 42i. The tangential stop 903 cooperates with a distal end structure 1080 (FIGS. 74 through 80) on the inserter 1046 to assure proper rotational alignment between the primary screw 42h, 42i and the inserter assembly 846. The configuration and function of the rotational alignment features are described in greater detail below attendant the discussion of the distal end structures 1080h and 1080i at FIGS. 74 and 77 through 80.

The radiused profile 916 of the distal face 914 enables the flange 132 to displace soft tissue surrounding the surgical incision that leads to the penetration site 624, rather than cutting or tearing the soft tissue. Displacement of tissue is less traumatic than cutting or tearing of tissue, for faster and less painful post-operative recovery. Also, when passing the primary screw 42h through the incision, the surgeon may rotate the primary screw in a direction opposite the cutting direction 109 of the threads 108 (e.g., in a counterclockwise direction for a right-handed thread) en route through the incision to reduce the cutting and tearing of soft tissue by the threads 108. Furthermore, the radiused profile 916 can enable tooling to access the body portion 102 of the primary screw 42h proximate the flange 132 to form the threads 108 in close proximity to the head portion 100.

Disposing the elongate side port(s) 112 closer to the tip junction 166 than to the proximal end 120 of the body portion 102 causes the elongate side port(s) 112 to traverse a longer axial distance during implantation than if the port(s) 112 were centered or disposed closer to the proximal end 120. The increased traversal length of the elongate side port(s) 112 may cause more live bone tissue to enter the primary screw 42h. Also, disposing the elongate side port(s) 112 closer to the tip junction 166 provides more engaged thread length of the external threads 108 near the proximal end 120 of the body portion 102, where the primary screw 42h engages with the denser cortical bone (e.g., at the exterior of the ilium). The enhanced engagement of the primary screw 42h with the cortical bone helps to securely anchor the primary screw 42h.

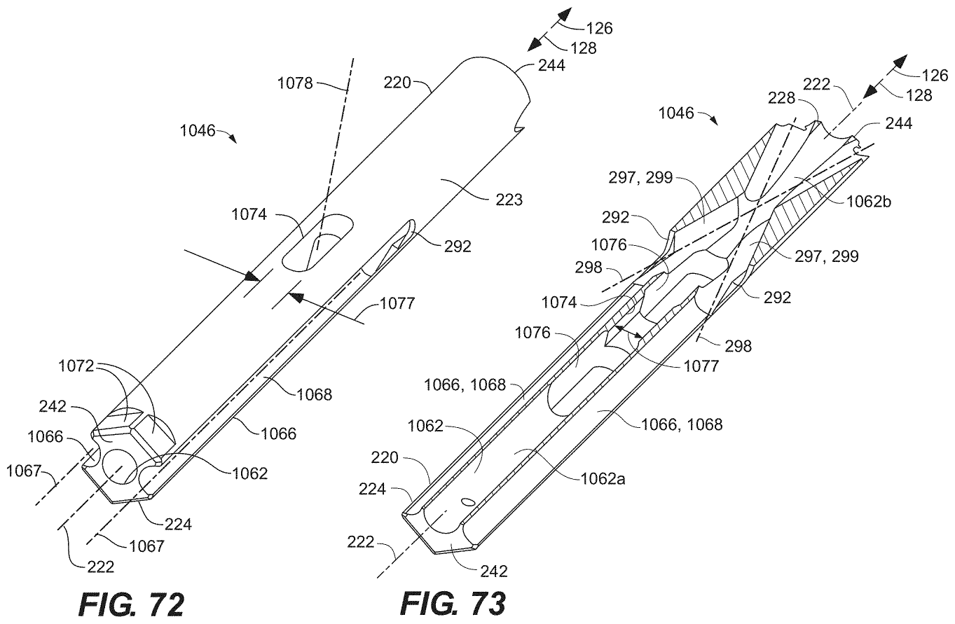

Referring to FIGS. 72 and 73, an inserter 1046 is depicted according to an embodiment of the disclosure. The inserter assembly 846 includes the inserter 1046 and an outer isolation sleeve 1048 (FIG. 56). The isolation sleeve 1048 includes dielectric properties, such as a polymer walls, a polymer coating, or an anodized surface. The isolation sleeve may be a tubular structure (depicted), or a thin film that is wrapped around or deposited onto the surface of the inserter 1046.

The inserter 1046 may include some of the same components and attributes as the inserter 46 (e.g., FIGS. 12A through 21), some of which are indicated by same-labeled reference characters. The inserter 1046 defines a central passage 1062 concentric about the inserter axis 222 that extends through the proximal face 242 and the distal face 244 of the inserter 1046. The central passage 1062 includes a proximal portion 1062a that extends into the inserter 1046 from the proximal face 242, and a distal portion 1062b that extends into the inserter 1046 from the distal face 244. The inserter 1046 may further define at least one access slot 1066, the access slot(s) 1066 each defining an access slot axis 1067 that is radially offset from, extends parallel to, and is coplanar with the inserter axis 222 and also coplanar with the canted axis or axes 298 of the side entrance port(s) 292. The access slot(s) 1066 defines an arcuate channel(s) 1068 that is accessible from a lateral side of the exterior surface 223 and terminates distally at the guide ramp(s) 299. In some embodiments, the proximal end 224 defines wrench flats 1072.

The inserter 1046 may define a key slot 1074 that bridges the proximal portion 1062a and the distal portion 1062b of the central passage 1062. The key slot 1074 extends laterally from one side of the main cylinder 220 and is centered about a key slot axis 1078 that intersects the inserter axis 222. A lateral width 1077 of the key slot 1074 is fabricated to a close tolerance (e.g., to within +/−200 micrometers of a specified dimension). The inserter 1046 may also define one or more extension slots 1076 that overlap the key slot 1074.

Functionally, the isolation sleeve 1048 insulates the metal inserter from the soft tissue to localize the (milliamp) electric current utilized for intraoperative neurophysiological monitoring (TOM). IOM is utilized to locate nerves/safeguard the patient from damage to nerves exiting the cauda equina (inferior extension of the spinal cord) that pass to the legs.

The arcuate channel(s) 1068 enable the side screw(s) 882 to be inserted into the access slot(s) 1066 anywhere along the length of the access slot(s) 1066. The wrench flats 1072 enable the inserter 1046 to be driven directly by a wrench tool, for example the multifunctional handle 888.

The extension slot(s) 1076 may form part of the proximal portion 1062a or distal portion 1062b (or both) of the central passage 1062. The extension slot(s) 1076 enable the central passage 1062 to be extended without need for special tooling. That is, if the inserter 1046 is of a length that prohibits reaching a mid-point of the inserter from a given end face 242, 244 with standard tooling, the central passage 1062 may be functionally extended by milling the key slot 1074 and, if necessary, the extension slot(s) 1076 for continuity of the central passage 1062. The key slot 1074 may also be configured to lock the primary screw driver 848 in a fixed rotational relationship, as described below.

Referring to FIG. 74, a distal end structure 1080h of the inserter 1046 for coupling to the primary screw 42h is depicted according to an embodiment of the disclosure. The distal end structure 1080h includes the boss 228 with exterior thread 232 for mating with the internal thread 138 of the primary screw 42h, akin to the assembly procedure described attendant to FIGS. 42 and 44. In addition, the distal end structure 1080h includes a tangential stop 1079 for engaging the tangential stop 903 of the primary screw 42h in the tangential direction. For the distal end structure 1080h, the tangential stop 1079 is located on the boss 228, taking the form of an abrupt termination 1082 at a distal end 1084 of the exterior thread 232. The abrupt termination 1082 engages the abrupt termination 905 at the distal end 907 of the interior thread 138 on the primary screw 42h to rotationally register and align the inserter 1046 relative to the primary screw 42h. The abrupt terminations 905, 1082 are configured to engage when the side screw port axes 152 and the canted axes 298 of the side entrance port(s) 292 are in alignment.

In some embodiments, the interior thread 138 of the primary screw 42h and the exterior thread 232 of the inserter 1046 are reverse threaded with respect to the external threads 108 of the primary screw 42h. That is, if the external threads 108 of the primary screw 42h are “right-handed” (i.e., the cutting rotational direction 109 is clockwise), the interior thread 138 of the primary screw 42h and the exterior thread 232 of the inserter 1046 may be “left handed” (i.e., engage fully by rotating counterclockwise).

Functionally, the reverse threaded coupling of the inserter 1046 relative to the external threads 108 of the primary screw 42h enables the inserter 1046 to be decoupled from the primary screw 42h by rotating the inserter 1046 in the cutting rotational direction 109. Resistance of the primary screw 42h within the bone in the cutting rotational direction 109 is greater than in an opposite “back out” rotational direction, so that the risk of altering the rotational alignment of the primary screw 42h (for example, because of binding or general resistance between the threads 138 and 232) is mitigated by decoupling the inserter 1046 in the cutting rotational direction 109.

Referring to FIGS. 77 through 80, a distal end structure 1080i of the inserter 1046 for coupling to the primary screw 42i is depicted according to an embodiment of the disclosure. Herein, the individual distal end structures 1080h and 1080i are referred to generically or collectively as distal end structures 1080. The distal end structure 1080i includes at least one lobe structure 1086 that extends in the distal direction 128 from the distal end 226 of the main cylinder 220. The lobe structure(s) 1086 may extend through a sectional plane 1088 proximate the junction of the lobe structure(s) 1086 and the main cylinder 220 (depicted), the sectional plane 1088 being normal to the inserter axis 222.

Each lobe structure 1086 includes a proximal neck portion 1090 and a distal head portion 1092. The proximal neck portion 1090 defines an axial thickness 1094 and a sectional plane perimeter 1096 that is coplanar with the sectional plane 1088. The sectional plane perimeter 1096 may be characterized as defining a tangential boundary 1098 and an outer radial boundary 1100 relative to the inserter axis 222. In some embodiments, the tangential boundary 1098 of the proximal neck portion 1090 defines the tangential stop 1079 of the inserter 1048 for engagement with the tangential stop 903 of the primary screw 42i. In the depicted embodiment, the tangential stop 1079 is a radial flat 1102 configured to engage the radial flat 912 of the primary screw 42i to rotationally register and align the inserter 1046 relative to the primary screw 42i.

The distal head portion 1092 is distal to the proximal neck portion 1090 and includes a distal face 1101. The distal face 1101 may be planar and engage the interior proximal face 137 of the primary screw 42i (depicted). In some embodiments, the distal head portion 1092 includes an oversized portion 1103 having an axial thickness 1104 and defining an oversized perimeter 1106. The oversized perimeter 1106 is dimensioned to pass axially through the access 932 and the axial thickness 1104 is dimensioned to provide a sliding fit within the undercut 934. The oversized perimeter 1106 extends radially beyond the outer radial boundary 1100 of the sectional plane perimeter 1096, tangentially beyond the tangential boundary 1098 of the sectional plane perimeter 1096, or both, to define a shoulder 1108.

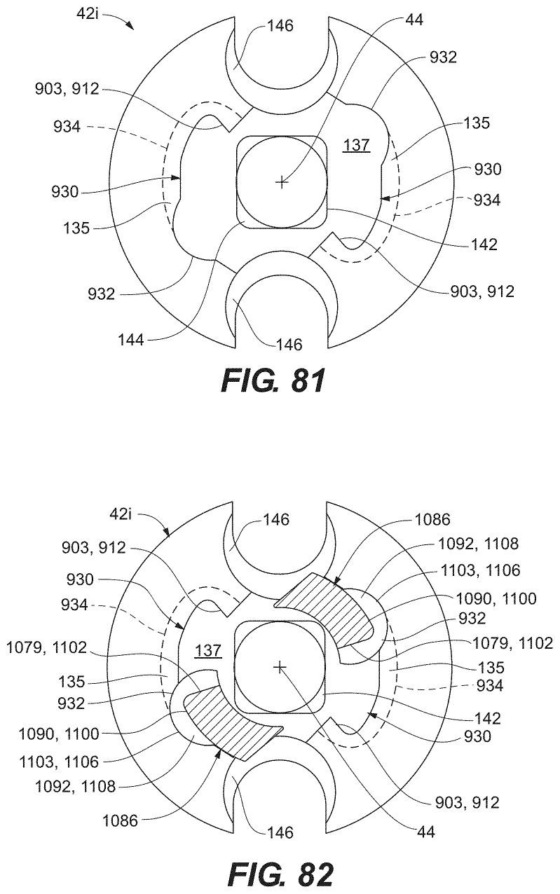

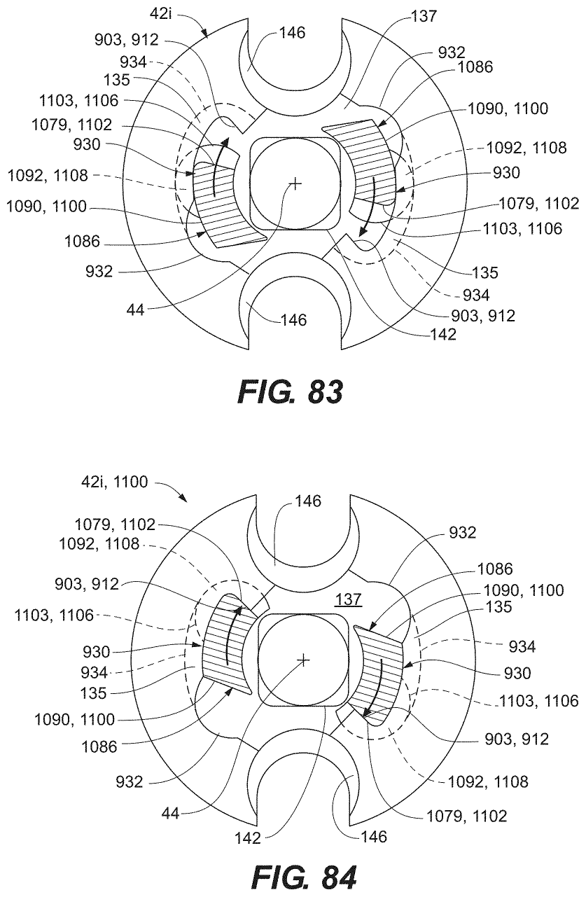

Referring to FIGS. 81 through 84, operational interaction between the lobe structures 1086 of the distal end structure 1080i of the inserter 1046 and the interlocking structures 930 of the primary screw 42i is depicted according to an embodiment of the disclosure. The primary screw 42i is depicted in isolation, prior to engagement with the lobe structures 1086 at FIG. 81, with the undercuts 934 depicted with hidden (broken) lines. The lobe structures 1086 of the distal end structure 1080i and the accesses 932 of the primary screw 42i are rotationally aligned, and the distal head portions 1092 of the lobe structures 1086 inserted into the accesses 932 and registered against the interior proximal face 137 of the primary screw 42i (FIG. 82). The distal end structure 1080i is rotated about the inserter axis 222 so that the distal head portions 1092 slide tangentially into the undercut 934 (FIG. 83). The distal end structure 1080i is rotated until the tangential stops 1079 of the lobe structures 1086 register against the stops 903 to define a fully interlocked orientation 1110 (FIG. 84).

Functionally, the distal head portions 1092 of the lobe structures 1086, when positioned within the undercuts 934, interact with the jut 936 to establish a fixed axial relationship between the inserter 1046 and the primary screw 42i along the axes 44 and 222. The tangential stops 903 and 1079 establish a fixed rotational relationship between the inserter 1046 and the primary screw 42i when the tangential stops 903 and 1079 are engaged in the fully interlocked orientation 1110. The tangential stops 903 and 1079 are configured to engage when the side screw port axes 152 and the canted axes 298 of the side entrance port(s) 292 are in alignment, for uninhibited passage of the side screws 882 through the inserter 1046 and into the side screw ports 146. The radial flats 912, 1102 provide a rigid stop mechanism that is not prone to displacement or compromise because of overtightening between the inserter 1046 and the primary screw 42i.

The lobe structures 1086 can also be utilized to verify that the side screws 882 are seated within the side screw ports 146 after implantation. Consider that the operational steps illustrated in FIGS. 81 through 84 are executed before the primary screw 42i and side screws 882 are set in the bone of the sacrum and ilium. Accordingly, the side screws 882 are driven into place when the lobe structures 1086 are in the fully interlocked orientation 1110. To remove the inserter 1046, the distal head portions 1092 are rotated from the fully interlocked orientation 1110 (FIG. 84) to the accesses 932 (FIG. 82) and withdrawn in the proximal direction 126 from the accesses 932. In rotating the distal head portion 1092 from the fully interlocked orientation 1110 into the accesses 932, the distal head portion 1092 may be configured so that the distal face 1101 of the distal head portion 1092 slides along the interior proximal face 137 of the primary screw 42i and over the side screw port 146, as depicted in FIG. 82. The primary screw 42i may be configured so that when the side screws 882 are seated within the side screw ports 146, the heads 410 of the side screws 882 are entirely distal to the interior proximal face 137 of the primary screw 42i. As such, if the distal head portion 1092 contacts the head 410 of the respective side screw 882 during removal of the inserter 1046, the operator knows that the side screw 882 is not properly seated within the side screw port 146 and can make corrective action.

Referring to FIGS. 75, 76, 85 and 86, the primary screw driver 848 and inserter 1046 and assembly are depicted in greater detail according to an embodiment of the disclosure. The primary screw driver 848 may include some of the components and attributes of the primary screw driver 48 (FIG. 24), some of which are indicated with same-labeled reference characters. The shaft portion 320 of the primary screw driver 848 includes a proximal portion 1112 and a distal portion 1114 separated by a mid-portion 1116. The proximal portion 1112 of the shaft portion 320 may include the wrench flats 330 as well as one or more tangential grooves 1118. Also, in the depicted embodiment, the driving head 336 at the distal end 328 of the primary screw driver 848 is a rounded-corner square for mating with the socket 142 of the primary screw 42h, 42i as depicted at FIGS. 76 and 86.

An exterior surface 1122 of the mid-portion 1116 defines a substantially polygonal cross-section 1124, such as a square 1126 (depicted). The polygonal cross-section 1124 defines a minimum radial outer dimension 1128 and a maximum radial outer diameter 1132 and may include rounded corners 1134. The minimum radial outer dimension 1128 may be fabricated to a close tolerance (e.g., to within +/−200 micrometers of a specified dimension) to provide a close-sliding fit of the mid-portion 1116 within the lateral width 1077 of the key slot 1074. In some embodiments, the clearance between the minimum radial outer dimension 1128 of the mid-portion 1116 and the lateral width 1077 of the key slot 1074 is in a range from 250 micrometers to 800 micrometers inclusive. The distal portion 1114 is dimensioned to pass through the key slot 1074 of the inserter 1046. The proximal portion 1062a of the central passage 1062 is sized to accommodate passage of the maximum radial outer diameter 1132 of the mid-portion 1116.

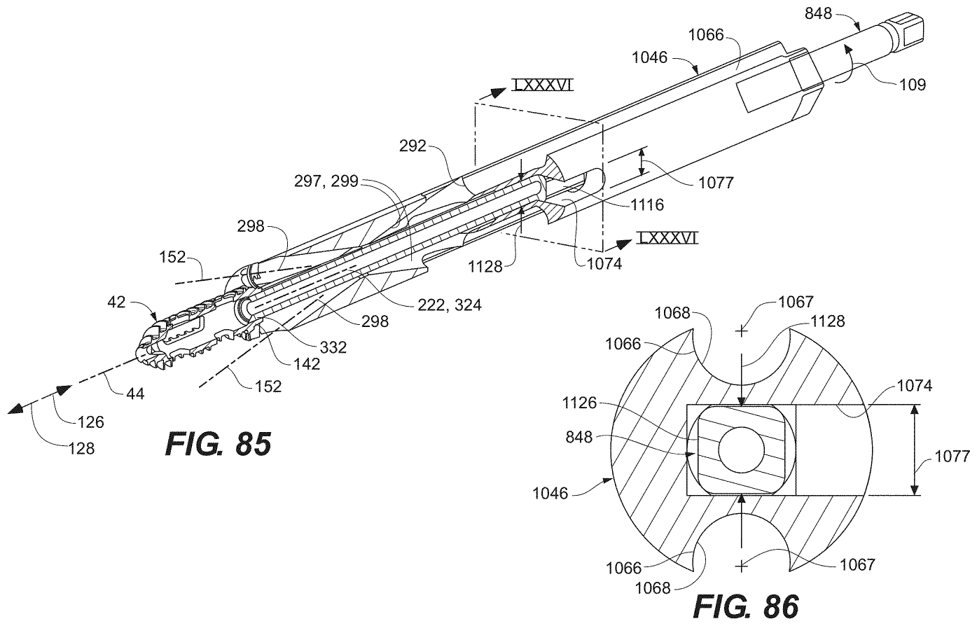

Functionally, the close, sliding fit between the minimum radial outer dimension 1128 of the mid-portion 1116 of the primary screw driver 848 and the lateral width 1077 of the key slot 1074 of the inserter 1046 establishes a substantially fixed rotational relationship between the primary screw driver 848 and the inserter 1046 when the primary screw 42 is secured to the inserter 1046 and the driving head 336 of the primary screw driver 848 is seated within socket 142 of the primary screw 42 (FIG. 85). The fixed rotational relationship assures that the inserter 1046 will rotationally follow the primary screw 42 as it is set into the bone, thereby assuring that the inserter 1046 remains aligned with the primary screw 42 (e.g., that each canted axis 298 of the inserter 1046 remains adequately aligned with the respective side screw port axis 152 of the primary screw 42). For embodiments where the threads 138, 232 are reverse threads, the primary screw driver 848 enables cutting rotation of the primary screw, because the reverse threads would disengage between the inserter 1046 and the primary screw driver 848 if the rotational relationship was not otherwise fixed. Also, maintaining the fixed rotational relationship generally prevents overtightening between the interior thread 138 on the primary screw 42h and the exterior thread 232 on the inserter 1046, the friction of which can cause rotational displacement of the primary screw 42 when the inserter 1046 is disconnected from the primary screw 42h.

The sizing of the proximal portion 1062a of the central passage 1062 to accommodate passage of the maximum radial outer diameter 1132 of the mid-portion 1116 enables the primary screw driver 848 to be inserted through the central passage 1062 for engagement of the mid-portion 1116 with the key slot 1074 of the inserter 1046. The tangential groove(s) 1118 can engage a detent (not depicted), such as a spring or ball plunger, to selectively fix the primary screw driver 848 in an axial location.

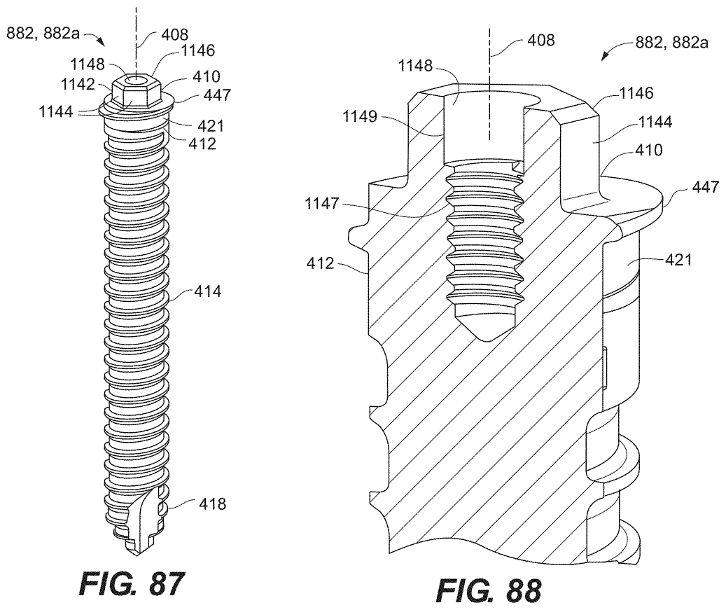

Referring to FIGS. 87 and 88, the side screw 882a is depicted in greater detail according to an embodiment of the disclosure. The side screw 882a may include some of the same components and attributes as the side screws 82 of FIGS. 36 through 37B, some of which are indicated with same-labeled reference characters. The head 410 of the side screw 882a includes a cap 1142 that protrudes proximally, the cap 1142 including wrench flats 1144. The cap 1142 may be dimensioned so that the wrench flats 1144 are radially inset from the flange 421 of the head 410. The wrench flats 1144 define a polygonal shape 1146 (hexagonal depicted). In some embodiments, the side screw 882a defines a tapped hole 1148 that is concentric about the side screw axis 408. The tapped hole 1148 includes a tapped portion 1147 and may include a clearance portion 1149 proximal to the tapped portion 1147.

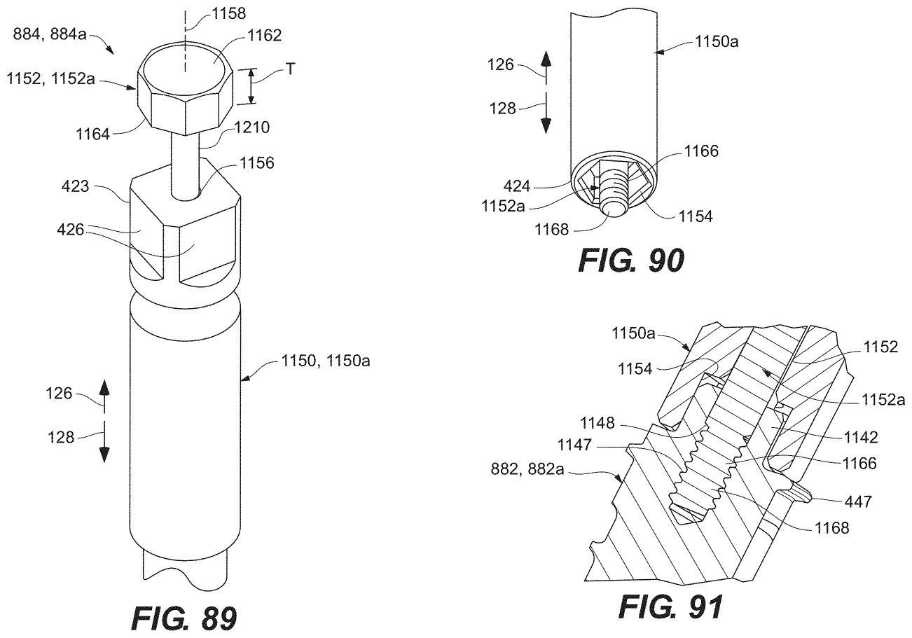

Referring to FIGS. 89 through 91, aspects of a side screw driver assembly 884a are depicted in greater detail according to an embodiment of the disclosure. The side screw driver assembly 884a includes a screw driver component 1150a and a screw retainer component 1152a. Herein, the side screws, the side screw driver assembly, the screw driver component, and the screw retainer component of FIG. 56 are referred to generically or collectively by the reference characters 882, 884, 1150, and 1152, respectively, and specifically or individually by the same reference characters followed by a letter suffix (e.g., side screw 882a, side screw driver assembly 884a, the screw driver component 1150a, and the screw retainer component 1152a).

The screw driver component 1150 may include some of the same components and attributes as the side screw driver 84 of FIGS. 30 and 31, some of which are indicated with same-labeled reference characters. For the depicted embodiment of the screw driver component 1150a, the flats 426 at the proximal end 423 define a square cross-section. The flats 426 of the screw driver component 1150a and the flats 330 of the primary screw driver 848 may be configured so that both components can be driven with the same tool. The distal end 424 of the screw driver component 1150a defines a socket 1154 configured to mate with the polygonal shape 1146 (hexagonal depicted). A through-passage 1156 extends along a rotation axis 1158 of the screw driver component 1150a.

The screw retainer component 1152a includes a draw rod 1210 having a polygonal head 1162 at a proximal end 1164 and a threaded portion 1166 at a distal end 1168. In the depicted embodiment, the polygonal head 1162 is octagon-shaped, but any suitable polygonal shape may be utilized. A diameter of the draw rod 1210 is dimensioned to slide through the through passage 1156 (FIG. 89), and a length of the draw rod 1210 is dimensioned so that the threaded portion 1166 extends into the socket 1154 beyond the distal end 424 of the screw driver component 1150a.

In assembly, the cap 1142 of the side screw 882a is inserted into the socket 1154 of the screw driver component 1150a. The screw retainer component 1152a is inserted into the through-passage 1156 of the screw driver component 1150a and the distal end 1168 engaged with the tapped hole 1148 of the side screw 882a. The threaded portion 1166 of the screw retainer component 1152a is threadably engaged with the tapped portion 1147 of the tapped hole 1148 to draw the side screw 882a into the socket 1154 and draw the polygonal head 1162 of the screw retainer component 1152a against the proximal end 423 of the screw driver component 1150a.

Functionally, the side screw driver assembly 884a secures the side screw 882a within the socket 1154 in a fully engaged configuration. The screw driver component 1150a is thereby aligned with the side screw 882a and the socket 1154 is optimally engaged with the cap 1142. The side screw 882a is thus secured to the side screw driver assembly 884a as it is passed through the inserter, to the implant site, and implanted. The optimal engagement between the cap 1142 and the socket 1154 prevents slippage therebetween when the screw driver component 1150a is torqued to implant the side screw 882a. Once the side screw 882a is implanted, the screw retainer component 1152a is decoupled from the side screw 882a by reversing rotation of the screw retainer component 1152a and withdrawing the screw driver component 1150a from the inserter 1046.

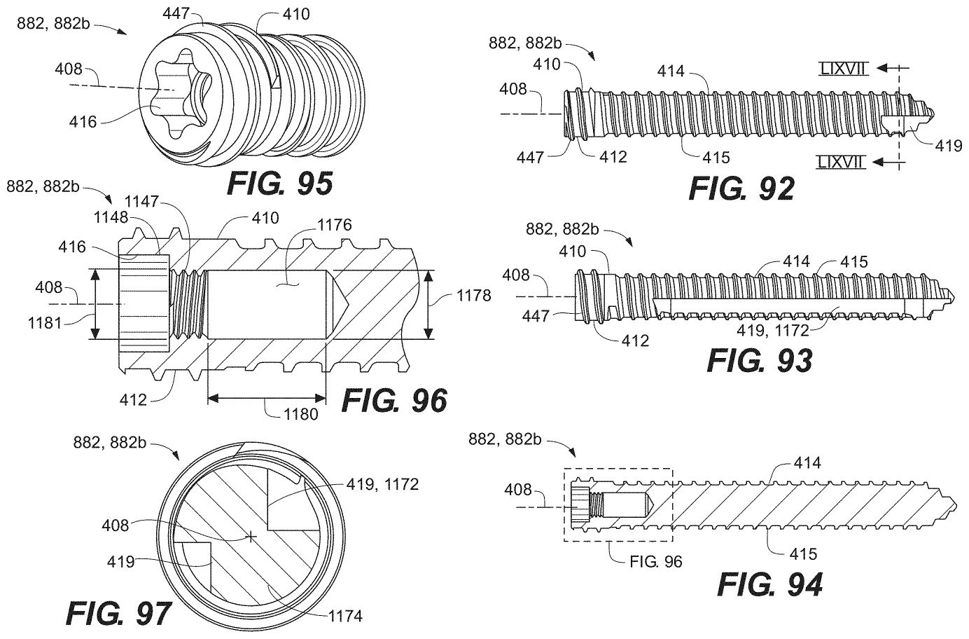

Referring to FIGS. 92 through 97, a side screw 882b is depicted according to an embodiment of the disclosure. The side screws 882b may include some of the same components and attributes as the side screws 82 and 882a, some of which are indicated with same-labeled reference characters. The side screw 882b is characterized having as at least one of the self-tapping flutes 419 as an elongated flute 1172 that extends over a majority of the threaded length of the side screw 882b (FIG. 93). In some embodiments, the lengthened self-tapping flute 419′ extends deeper into a cross-section 1174 of the side screw 882b (FIG. 97). The side screw 882b may include a clearance cavity 1176 distal to the tapped hole 1148 of the head 410 (FIGS. 94 and 96), the clearance cavity 1176 defining a clearance diameter 1178 that extends along an axial length 1180 and parallel to the side screw axis 408. The socket 416 of the side screw 882b is depicted as including a hexalobular (e.g., TORX®) internal drive feature (FIG. 95), but, as with the other side screws 82 and 882, may define any one of a variety of shapes, such as a triangle, rectangle, square, hexagon, octagon, cross, or other shapes suitable for torsional driving of the side screw 882b.

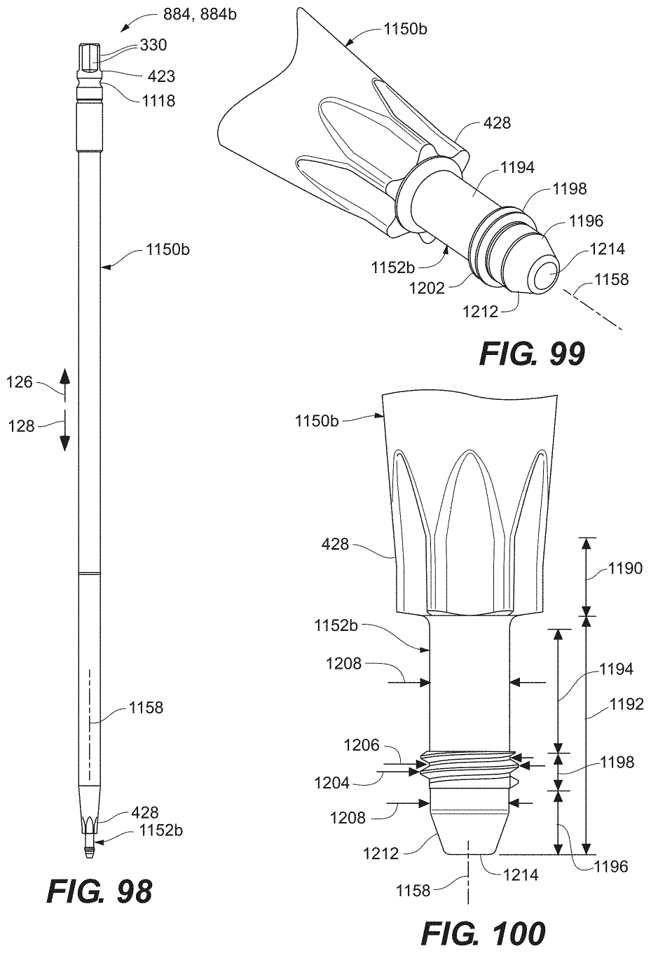

Referring to FIGS. 98 through 100, a side screw driver assembly 884b is depicted according to an embodiment of the disclosure. The side screw driver assembly 884b includes a screw driver component 1150b and a screw retainer component 1152b. In the depicted embodiment, the screw driver component 1150b and the screw retainer component 1152b are unitary, with the screw retainer component 1152b being in a fixed relationship with and extending distally from the screw driver component 1150b. The screw driver component 1150b may include some of the same components and attributes as the screw driver component 1150a and the side screw driver 84, some of which are indicated with same-labeled reference characters. The driving head 428 of the screw driver component 1150b is depicted as hexalobular (e.g., TORX®), but, as with the other driving heads 428, may define any one of a variety of cross-sections, such as a triangle, rectangle, square, hexagon, octagon, cross, or other shapes suitable for torsional driving of the side screw 882b. The driving head 428 may be configured to define a maximum seating depth 1190.

The screw retainer component 1152b extends distally along the rotation axis 1158 of the side screw driver assembly 884b to define an axial length 1192 beyond the driving head 428 of the screw driver component 1150b. The axial length 1192 includes an unthreaded base length 1194 and an unthreaded lead length 1196 separated by a threaded length 1198. The threaded length 1198 includes threads 1202 that define a crest diameter 1204 and a root diameter 1206 and are configured to threadably engage the tapped portion 1147 of the tapped hole 1148 of the side screw 882b. The unthreaded lengths 1194 and 1196 each define a clearance diameter 1208 that may be less than or equal to the root diameter 1206. The unthreaded lead length 1196 may define a taper 1212 that tapers toward the rotation axis 1158 at a distal end 1214.

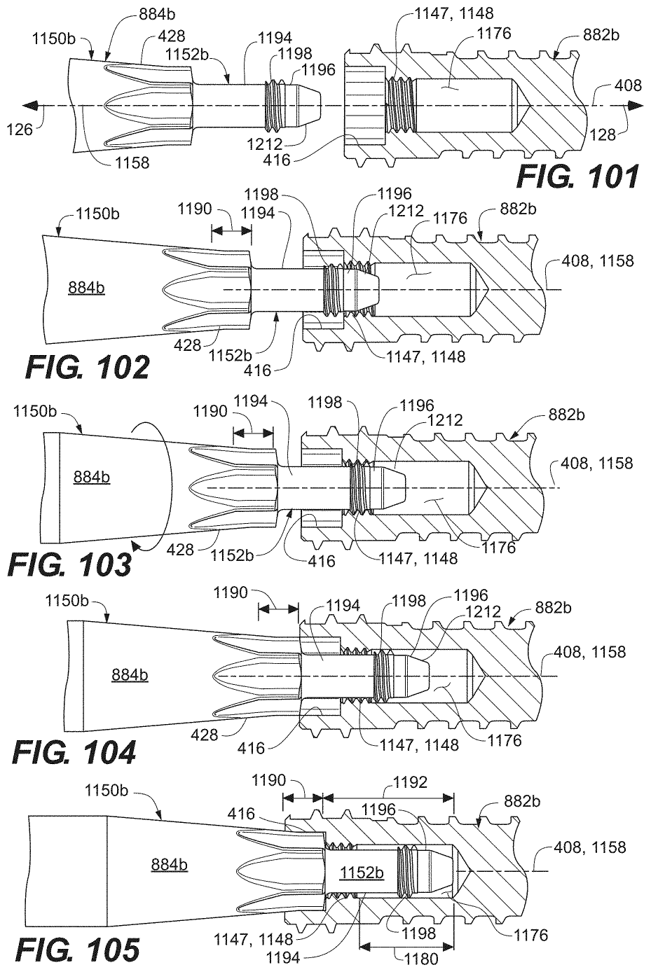

Referring to FIGS. 101 through 105, operation of the side screw driver assembly 884b with the side screw 882b is depicted according to an embodiment of the disclosure. The side screw 882b may be secured as depicted at FIGS. 101 through 105 prior to insertion into the inserter 1046, 46 (i.e., prior to insertion into the patient). While depicted and described as being used with the inserters 1046, the side screw driver assembly 884 may be utilized with the inserter 46 also.

The side screw driver assembly 884b is positioned proximal to the side screw 882b with the side screw axis 408 of the side screw driver assembly 884b and the rotation axis 1158 of the side screw driver assembly 884b in substantial alignment (FIG. 101). The side screw 882b and driver assembly 884b are brought together so that the lead length 1196 of the screw retainer component 1152b is within the tapped hole 1148 of the side screw 882b (FIG. 102). The side screw 882b and driver assembly 884b are rotated about one or both of the respective axes 408, 1158 to threadably engage the threaded length 1198 of the screw retainer component 1152b with the tapped portion 1147 of the tapped hole 1148 of the side screw 882b (FIG. 103). The relative rotation between the side screw 882b and driver assembly 884b continues until the threaded length 1198 of the screw retainer component 1152b clears the tapped portion 1147 of the tapped hole 1148 of the side screw 882b, such that the threaded length 1198 of the screw retainer component 1152b is disposed within the clearance cavity 1176 (FIG. 104). The side screw 882b and driver assembly 884b are pushed together and rotated as necessary so that the unthreaded base length 1194 slides through the tapped portion 1147 of the tapped hole 1148, thereby seating the driving head 428 of the screw driver component 1150b within the socket 416 of the side screw 882b to define a driving configuration for the side screw driver assembly 884b and the side screw 882b (FIG. 105).

After the side screw 882b is set, the driving head 428 of the screw driver component 1150b can be withdrawn from the socket 416 (FIG. 104). The threaded length 1198 of the screw retainer component 1152b can be reversed through tapped portion 1147 of the tapped hole 1148 (FIG. 103), and the side screw driver assembly 884b withdrawn from the side screw 882b. In some embodiments, the threads of the threaded length 1198 and the tapped portion 1147 are reverse threaded relative to the threads of the threaded shaft 414 of the side screw 882b.

For embodiments where the driving head 428 defines the maximum seating depth 1190, the socket 416 is configured so that the driving head 428 is separated from or in light contact with the tapped portion 1147 of the tapped hole 1148 when the driving head 428 is firmly seated within the socket 416. For the hexalobular driving head of FIGS. 99 and 100, the maximum seating depth 1190 is established by flaring of the driving head 428 and tangential widening of the hexalobes. In some embodiments, the combined axial length 1180 of the clearance cavity 1176 and the tapped portion 1147 of the tapped hole 1148 is long enough to accept the axial length 1192 of the screw retainer component 1152b without the lead length 1196 or the distal end 1214 of the screw retainer component 1152b contacting a distal end of the clearance cavity 1176.

Functionally, the screw retainer component 1152b assures that the side screw 882b remains coupled to the side screw driver assembly 884b. Should the side screw 882b become dislodged from the driving head 428, the interference between the tapped portion 1147 of the tapped hole 1148 and the threaded length 1198 of the screw retainer component 1152b provides loose coupling between the side screw driver assembly 884b and the side screw 882b, enabling the operator to reseat the driving head 428 within the socket 416. The interference between the tapped portion 1147 and the threaded length 1198 also enables retrieval of the side screw 882b should the need arise.

The threads of the tapped portion 1147 of the tapped hole 1148 and the threaded length 1198 of the screw retainer component 1152b may function only to provide the aforementioned loose coupling between the side screw driver assembly 884b and the side screw 882b. Accordingly, in some embodiments, the lengths of the threads for the tapped portion 1147 and the threaded length 1198 need only be enough to provide the interference as stated while preventing inadvertent decoupling during the rigors of insertion and implantation. In some embodiments, the rotational length of one or both of the tapped portion 1147 and the threaded length 1198 is between 1 and 4 turns inclusive; in some embodiments, between 2 and 4 turns. In the depicted embodiment, the tapped portion 1147 of the side screw 882b is configured for about 3.5 turns while the threaded length 1198 of the screw retainer component 1152b is configured for about 2.5 turns.

With the screw retainer component 1152b located distal to and being unitary with the screw driver component 1150b, none of the cross-section of the screw driver component 1150b is removed to provide passage of the screw retainer component 1152 therethrough. Also, the presence of the screw retainer component 1152b does not reduce the material available for the driving head 428. Accordingly, the torsional cross-section of the screw driver component 1150b and driving head 428 is not compromised by the screw retainer component 1152b, which also enables the screw retainer component 1152b to be larger and stronger. Also, because the screw retainer component 1152b does not extend through the proximal end 423 of the screw driver component 1150b, the risk of breaking the screw retainer component 1152 by inadvertently grabbing and over-torqueing the screw retainer component 1152b with a torqueing device (e.g., handles 88 or 888) is eliminated.

The taper 1212 at the distal end 1214 of the screw retainer component 1152b serves as a pilot for initiating the threading of the threaded length 1198 through the tapered portion 1147. In embodiments where the threaded length 1198 and the tapped portion 1147 are reverse threaded relative to the threads of the threaded shaft 414 of the side screw 882, there is more torsional resistance to overcome the threaded seating of the side screw 882 when decoupling the screw retainer component 1152 from the tapped hole 1148 than for a non-reversed thread. Accordingly, the chance of partially dislodging the side screw 882 from a set depth during decoupling is reduced.

The elongated flute(s) 1172 is effectively a V-shaped version of the side cavity 425.2 that defines the side window 425.6 of side screw 82′ and functions as described attendant to FIGS. 28A and 28B. Embodiments where the elongated flute 1172 extends further into the cross-section 1174 provides a larger cross-section for enhanced bone tissue ingrowth. In some embodiments, there is only one elongated flute 1172, so that the deeper cut of the elongated flute 1172 does not compromise the torsional strength of the side screw 882b while providing the enhanced bone tissue ingrowth.

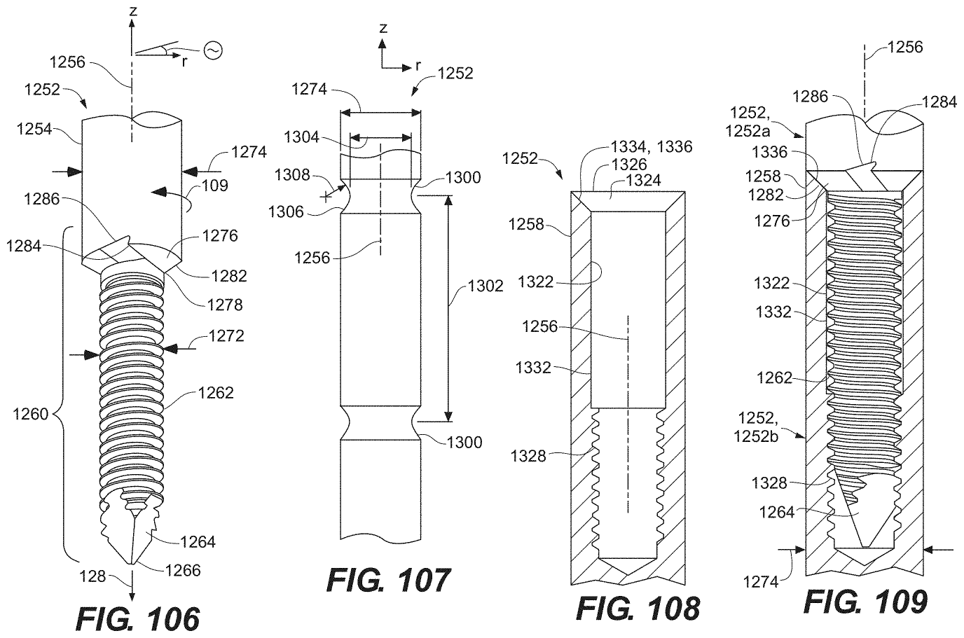

Referring to FIGS. 106 through 109, aspects of the bifurcated guide rod 852 are depicted in greater detail according to an embodiment of the disclosure. The bifurcated guide rod 852 includes two identical segments 1252a and 1252b, as depicted in FIG. 1, that can be joined together to form the bifurcated guide rod 852 of extended length. The segments 1252a and 1252b are referred to generically and collectively as segment(s) 1252. Each segment 1252 includes shaft 1254 that extends along a centerline axis 1256 and having a proximal end 1258 and a distal end 1260. The distal end 1260 includes a male threaded portion 1262 having a self-tapping pilot tip 1264 at a distal extremity 1266 of the segment 1252. In some embodiments, a crest diameter 1272 of the male threaded portion 1262 is reduced relative to a shaft diameter 1274 to define a shoulder 1276 at a proximal end 1278 of the male threaded portion 1262. The shoulder portion 1276 may define a tapered surface 1282 that slopes toward the centerline axis 1256 in the distal direction 128. In some embodiments, the tapered surface 1282 includes a cutting tooth 1284. The cutting tooth 1284 may be defined by a flute 1286 that is recessed into the tapered surface 1282. In some embodiments, each segment 1252 includes a plurality of such teeth 1284.

In some embodiments, the shaft 1254 includes a plurality of tangential grooves 1300. The tangential grooves 1300 are centered at uniform intervals 1302 of known length along the centerline axis 1256. Each groove 1300 reduces the cross-section of the shaft 1254 to a minor diameter 1304, and may define a circular radial profile 1306 defined by a radius 1308. In some embodiments, the uniform intervals 1032 are within a range between a minimum implant length and a maximum implant length. In some embodiments, the uniform intervals 1302 are within a range of 5 millimeters to 30 millimeters inclusive. In some embodiments, the minor diameter 1304 is in a range of 2 millimeters to 3 millimeters inclusive, and the radius 1308 is in a range of 1 to 2 millimeters inclusive.

The proximal end 1258 defines a socket 1322 that extends along the centerline axis 1256, accessible from an opening 1324 at a proximal extremity 1326 of the segment 1252. The socket 1322 includes a female threaded portion 1328 with threads configured to mate with the male threaded portion 1262 of the distal end 1256. The socket 1322 may also include a clearance diameter portion 1332 proximal to the female threaded portion 1328, as well as a countersink portion 1334 that extends distally from the opening 1324. The countersink portion 1334 defines a sloped surface 1336 that matches the slope of the tapered surface 1282.

Functionally, the shorter lengths of the individual segments 1252 enable an initial penetrating force to be applied on the segment 1252 with less risk of buckling the segment 1252. Once the tapping of the bone is started, the segments 1252a and 1252b can be joined together to form the bifurcated guide rod 852 of extended length. The male and female threaded portions 1262 and 1328 enable the segments 1252 to be joined together for the length extension. By making the segments 1252 identical, the operator does not have to discern between the segments 1252 when starting the initial segment 1252.

The tangential grooves 1300 can be fluoroscopically visualized during surgery. The visualization enables identification of reference points along the bifurcated guide rod 852. The uniform intervals 1302 can provide an indication of lengths in situ proximate the implant site.

The reduced crest diameter 1272 of the male threaded portion 1262 relative to a shaft diameter 1274 enables the segments 1252a and 1252b to be joined together while maintaining the shaft diameter 1274 uniformly along the length of the bifurcated guide rod 852. The clearance diameter portion 1332 enables engagement of an adequate number of threads between the male and female threaded portions 1262 and 1328 (e.g., four or five full turns) without need for an excessive number of turns that would be required to engage the full length of the male threads 1262. The countersink portion 1334 accommodates the tapered surface 1282 of the adjoining segment 1252 while maintaining the uniform shaft diameter of the bifurcated guide rod 852. The tapered surface 1282 of the shoulder portion 1276 facilitates passage of the shoulder portion 1276 through bone tissue with less resistance than would a flat shoulder. The cutting tooth or teeth 1284 augments a cutting action during rotational advancement of the segment 1252 that eases the passage through the bone tissue, particularly the exterior cortical bone tissue. Use of either the tapered surface 1282 or the cutting tooth or teeth 1284, but not both, is also contemplated.

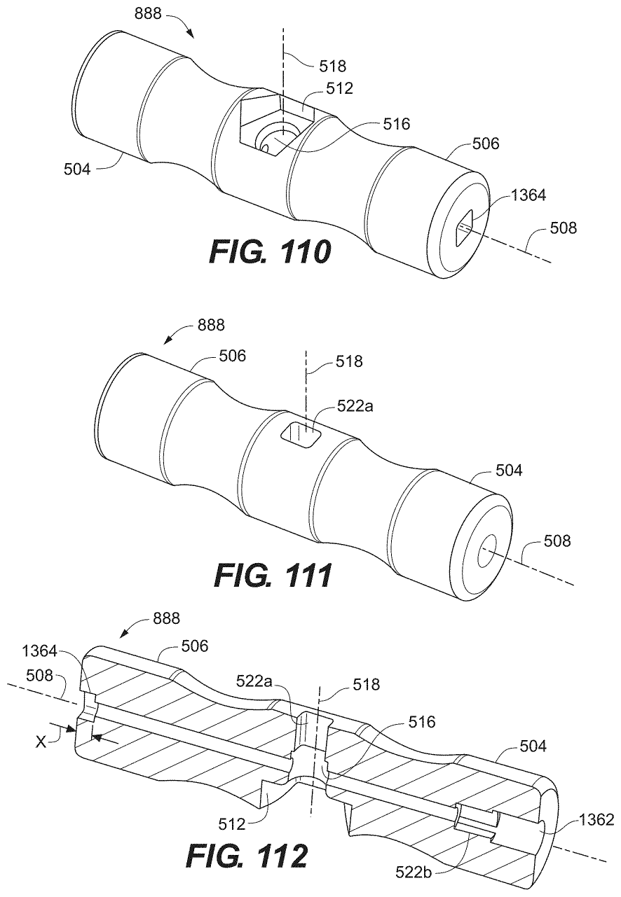

Referring to FIGS. 110 through 112, the multifunctional handle 888 is depicted in greater detail according to an embodiment of the disclosure. The multifunctional handle 888 includes some of the same components and attributes as the multifunctional handle 88, some of which are indicated by same-labeled reference characters. The socket 512 is sized to mate with the wrench flats 1072 at the proximal end 224 of the inserter 1046. The through-aperture 516 is configured to define a first socket 522a for driving the primary screw driver 848 and screw driver components 1150, the socket 522a being opposite the socket 512. A second socket 522b, also for driving the primary screw driver 848 and screw driver components 1150, may be defined at the end of the first handle portion 504, such that the second socket 522b is concentric about the lateral axis 508. In some embodiments, the second socket 522b is recessed with a clearance hole lead-in 1362.

For the multifunctional handle 888, a polygonal cavity 1364 may be defined for driving the screw retainer component 1152a. The polygonal cavity 1364 is configured to mate with the polygonal head 1162 of the screw retainer component 1152a. The polygonal cavity 1364 may be disposed at the end of the second handle portion 506, concentric with the lateral axis 508. In some embodiments, the polygonal cavity 1364 extends into the second handle portion 506 at an effective depth X that is less than an axial thickness T of the polygonal head 1162. In some embodiments, the polygonal cavity 1364 and polygonal head 1162 are sized to be substantially smaller than the sockets 522a and 522b, so that the polygonal head 1162 cannot be inadvertently driven by the sockets 522a and 522b.

It is noted that, for one component to be “shaped to mate with” or “configured to mate with” another component, as used herein, does not require that the same polygonal shape. For example, the polygonal cavity 1364 is configured or shaped to mate the polygonal head 1162, even though the polygonal cavity 1364 is depicted as square and the polygonal head 1162 is depicted as octagonal. That is, a square socket is capable of driving an octagonal cap. Accordingly, the “shaped or configured to mate with” description does not require the same polygonal type.

Functionally, the presence of the two sockets 522a and 522b enables alternative configurations for driving the primary screw driver 848 and side screw drivers, 1150. By using socket 522a, the multifunctional handle 888 is configures as a T-handle for the screw drivers 884, 1150. By using socket 522b, the multifunctional handle 888 is configures as an axially-extending driver handle for the screw drivers 884 and 1150. The clearance hole lead-in 1362 for the second socket 522b as well as the through-aperture 516 for the first socket 522a can provide a bearing surface against the shafts 320 and 419 of the screw drivers 848 and 1150 for stability. By dimensioning the effective depth X of the polygonal cavity 1364 to be less than the axial thickness T of the polygonal head 1162 of the screw retainer component 1152a, the polygonal cavity 1364 couples only to the polygonal head 1162, avoiding accidental coupling with the flats 426 of the screw driver component 1150a.

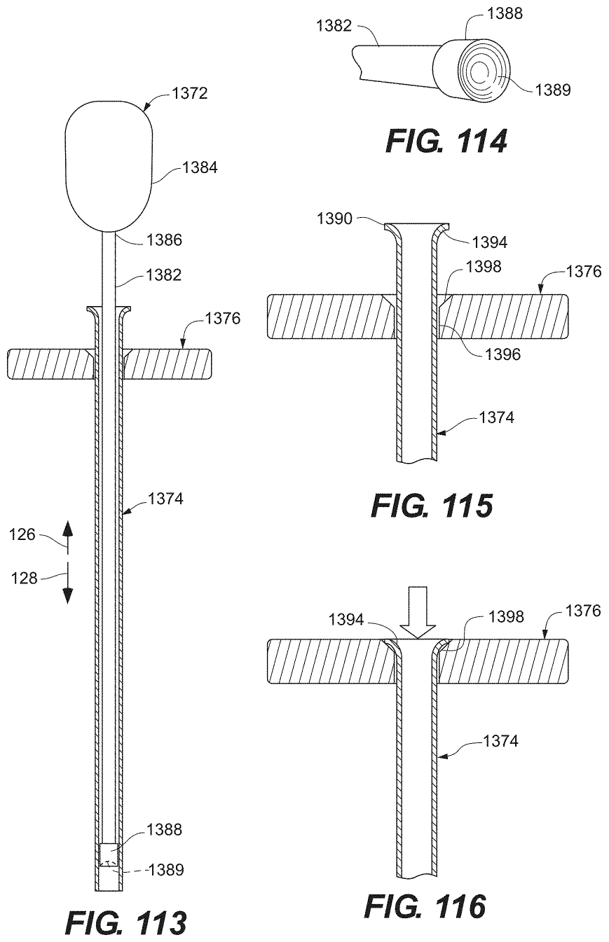

Referring to FIGS. 113 through 116, the plunger assembly 886 is depicted in greater detail according to an embodiment of the disclosure. The plunger assembly 886 includes a plunger 1372, a plunger tube 1374, and a pallet disk 1376. The plunger 1372 includes a plunger stem 1382 and a plunger handle 1384 at a proximal end 1386 thereof. In one embodiment, the plunger handle 1384 is of a bulbous shape (depicted). A distal end 1388 of the plunger stem 1382 may define a cupped recess 1389. In some embodiments, the distal end 1388 of the plunger stem 1382 is dimensioned for a close sliding fit with an inner diameter 1393 of the plunger tube 1374.

The plunger tube 1374 includes a proximal end 1390 and a distal end 1392 and defines the inner diameter 1393. In some embodiments, a flared portion 1394 is formed at the proximal end 1390 of the plunger tube 1374. The pallet disk 1376 defines a center hole 1396 sized for passage of the plunger tube 1374 therethrough. The center hole 1396 may include a chamfered inlet 1398.

In assembly, the plunger tube 1374 is inserted through the center hole 1396 so that the flared portion 1394 registers within the chamfered inlet 1398. The chamfered inlet 1398 and the flared portion 1394 may be dimensioned so that, when the flared portion 1394 is registered within the chamfered inlet 1398, the plunger tube 1374 is substantially flush with the pallet disk 1376 (FIG. 116).

In operation, the plunger tube 1374 is inserted through the inserter 1046 so that the distal end 1392 is coupled to the aperture 902 of the interior chamber 110 of the primary screw 42. Biologic or other bone grafting material may be placed on the pallet disk 1376 and a portion of the biologic fed into the flared portion 1394 of the plunger tube 1374. The plunger stem 1382 is inserted into the plunger tube 1374 so that the cupped recess 1389 pushes the biologic through the plunger tube 1374 and into the primary screw 42. The close sliding fit between the distal end 1388 of the plunger stem 1382 and the inner diameter 1393 of the plunger tube 1374 may sweep the inner diameter 1393 clean as the plunger stem 1382 pushes the biologic toward the primary screw 42. The plunger stem 1382 is withdrawn from the plunger tube 1374, followed by more biologic loaded into the flared portion 1394 from the pallet disk 1376. The steps of loading the biologic, pushing the biologic into the primary screw 42 with the plunger stem 1382, and withdrawing the plunger stem 1382 from the plunger tube 1374 is repeated until implant site has been sufficiently loaded with biologic. The plunger 1372 may also be used to pack or tamp the biologic within the primary screw 42 by tapping or pounding on the handle 1384 with a hand, fist, or mallet.

In some embodiments, the various foregoing methods are outlined at least in part on the instructions 892. The instructions 892 may be physically included with the kit 840 such as on a printed document (depicted), compact disc, or flash drive. In other embodiments, the instructions 892 are provided remotely, for example on a hard drive of a remote server that is internet accessible with an electronic device such as a computer, smart phone, or electronic tablet. The instructions 892 may include text, photos, videos, or a combination thereof to instruct and guide the user.

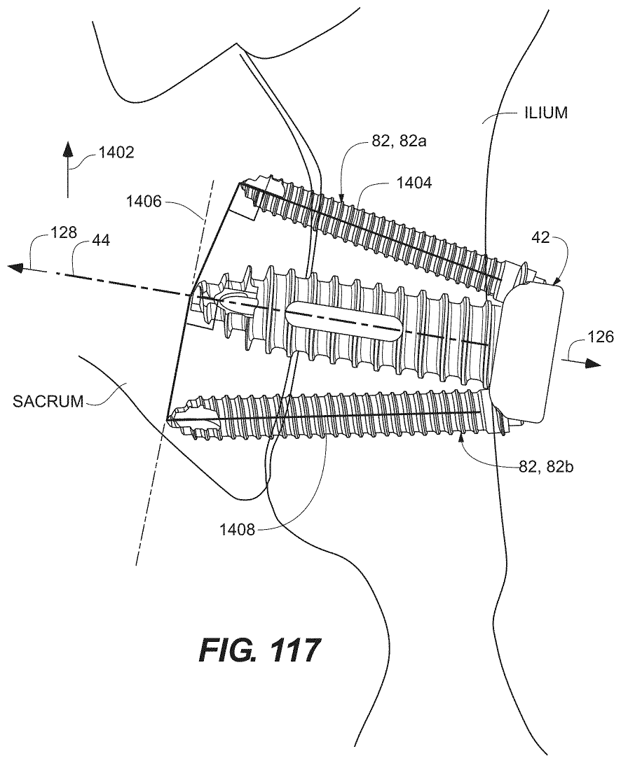

Referring to FIG. 117, an example implantation of the primary screw 42 is depicted according to an embodiment of the disclosure. In this example, the distal direction 128 central axis 44 of the primary screw 42 toward a superior direction 1402 so that the primary screw 42 penetrates substantially normal to the tissue layer 626. Also in this depiction, the cross-section is slightly skewed (not parallel with) the coronal plane, so that the cross section of the ilium is thicker than in FIG. 55.

For the FIG. 117 implant, a superior side screw 82a of the side screws 82 is shorter than an inferior side screw 82b of the side screws. The superior side screw 82a is shortened to avoid impinging nerves that reside in the superior region of the sacrum. The shorter superior side screw 82a is still effective because there is less ilium thickness to traverse to reach the cortical bone of the sacrum. The inferior screw 82b is longer because there is more local thickness of the ilium at the inferior location and there is more bone to engage in the sacrum.

In some embodiments, a threaded length 1404 of the superior side screw 82a is the same as the threaded length of the primary screw 42. Because the superior side screw 82a extends at the acute angle θ1, the distal end 418 of the superior side screw 82a does not reach a depth plane 1406 of the primary screw 42, the “depth plane” 1406 being at the distal extremity 105 of the primary screw 42 and orthogonal to the central axis 44. In some embodiments, a threaded length 1408 of the inferior screw 82b is dimensioned to reach the depth plane 1406 at the acute angle θ1.

Each of the additional figures and methods disclosed herein can be used separately, or in conjunction with other features and methods, to provide improved devices and methods for making and using the same. Therefore, combinations of features and methods disclosed herein may not be necessary to practice the disclosure in its broadest sense and are instead disclosed merely to particularly describe representative and preferred embodiments.

Various modifications to the embodiments may be apparent to one of skill in the art upon reading this disclosure. For example, persons of ordinary skill in the relevant arts will recognize that the various features described for the different embodiments can be suitably combined, uncombined, and re-combined with other features, alone, or in different combinations. Likewise, the various features described above should all be regarded as example embodiments, rather than limitations to the scope or spirit of the disclosure.

Persons of ordinary skill in the relevant arts will recognize that various embodiments can comprise fewer features than illustrated in any individual embodiment described above. The embodiments described herein are not meant to be an exhaustive presentation of the ways in which the various features may be combined. Accordingly, the embodiments are not mutually exclusive combinations of features; rather, the claims can comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Unless indicated otherwise, references to “embodiment(s)”, “disclosure”, “present disclosure”, “embodiment(s) of the disclosure”, “disclosed embodiment(s)”, and the like contained herein refer to the specification (text, including the claims, and figures) of this patent application that are not admitted prior art.

For purposes of interpreting the claims, it is expressly intended that the provisions of 35 U.S.C. 112(f) are not to be invoked unless the specific terms “means for” or “step for” are recited in the respective claim.

1.-112. (canceled)

113. A bone screw for fusion of a sacroiliac joint, comprising a body portion having a side wall concentric about a central axis and defining an interior chamber, said body portion including external threads and defining a first side port that extends through said side wall and is in fluid communication with said interior chamber, said body portion defining a laterally extending mid-plane that is coplanar with said central axis, said first side port being centered about a first axis that extends in a first lateral direction and is parallel to and offset from said laterally extending mid-plane.

114. The bone screw of claim 113, wherein said first side port is elongate in an axial direction.

115. The bone screw of claim 114, wherein:
said first side port includes an edge wall that defines an external opening, said edge wall having a plurality of tangential edges including an elongate leading tangential edge and an elongate trailing tangential edge relative to a rotational direction for setting said primary screw into bone; and
said edge wall adjacent said elongate trailing edge defines an acute sweeping angle relative to said laterally extending mid-plane, said acute sweeping angle being open in a radially inward direction.

116. The bone screw of claim 114, wherein:
said body portion defines a second side port that extends through said side wall and is in fluid communication with said interior chamber;
said second side port is centered about a second axis that extends in a second lateral direction and is parallel to and offset from said laterally extending mid-plane;
said second lateral direction is opposite said first lateral direction; and
said first axis and said second axis are offset from said laterally extending mid-plane in opposite directions.

117. The bone screw of claim 116, wherein:
said first side port includes a first edge wall that defines a first external opening, said first edge wall including a plurality of tangential edges;
said second side port includes a second edge wall that defines a second external opening, said second edge wall including a plurality of tangential edges; and
one of said plurality of tangential edges of said first edge wall and one of said plurality of tangential edges of said second edge wall are coplanar.

118. The bone screw of claim 117, wherein said one of said plurality of tangential edges of said first edge wall and said one of said plurality of tangential edges of said second edge wall that are coplanar are leading elongate tangential edges relative to a rotational direction for setting said primary screw into bone.

119. The bone screw of claim 117, wherein:
said body defines a first tangential notch that extends tangentially from one of said plurality of tangential edges of said first edge wall; and
said body defines a second tangential notch that extends tangentially from one of said plurality of tangential edges of said second edge wall.

120. The bone screw of claim 119, wherein said first tangential notch and said second tangential notch extend in opposite tangential directions.

121. The bone screw of claim 117, wherein:
said body defines a first axial notch that extends axially from said first edge wall;
said body defines a second axial notch that extends axially from said second edge wall; and
said first axial notch and said second axial notch extend in a same axial direction.

122. The bone screw of claim 113, wherein said body portion includes a tip portion, said external threads of said tip portion including a root portion that defines radii perpendicular to said central axis that decrease along said central axis in a distal direction so that said root portion of said external threads of said tip portion defines a tapered profile that tapers toward said central axis in said distal direction, said tip portion defining said first side port.

123. The bone screw of claim 122, wherein said first axis projects onto said central axis in a direction orthogonal to said laterally extending mid-plane to define an acute projected angle between said first axis and said central axis, said acute projected angle being open to said distal direction.

124. The bone screw of claim 123, wherein said tip portion defines a flute, said first axis extending through said flute.

125. The bone screw of claim 124, wherein said tip portion includes a distal extremity that extends about said central axis to define an opening that is in fluid communication with said interior chamber.

126. The bone screw of claim 125, wherein said distal extremity includes an arcuate segment that extends along a distal extremity plane.

127. The bone screw of claim 126, wherein said distal plane is orthogonal to said central axis.

128. The bone screw of claim 127, wherein said tip portion includes a cutting tooth that extends to said distal extremity plane.

129. The bone screw of claim 128, wherein said cutting tooth is formed by a relief slot defined by said tip portion, said relief slot extending proximally from said distal extremity and being open to said distal extremity plane, said slot defining an elongate axis that extends in said distal direction.

130. The bone screw of claim 129, wherein said cutting tooth defines an acute angle.
